(19) United States
(12) Patent Application Publication
(10) Pub. No.: US 2022/0289837 A1
(43) Pub. Date: Sep. 15, 2022

(54) Cystic Fibrosis Transmembrane Conductance Regulator Stabilizing Agents

(71) Applicants: VIB VZW, gent (BE); VRIJE UNIVERSITEIT BRUSSEL, Brussel (BE); UNIVERSITÉ LIBRE DE BRUXELLES, Bruxelies (BE); KATHOLIEKE UNIVERSITEIT LEUVEN, K.U.LEUVEN R&D, Leuven (BE)

(72) Inventors: Jan Steyaert, Beersel (BE); Els Pardon, Wezemaal (BE); Toon Laeremans, Dworp (BE); Cedric Govaerts, Uccle (BE); Magdalena Grodecka, Lody (PL); Maud Sigoillot, Ixelles (BE); Marie Overtus, Watermael-Boitsfort (BE); Marianne Sylvia Carlon, Holsbeek (BE); Marjolein Ensinck, Heverlee (BE); Abel Garcia-Pino, Brussel (BE)

(21) Appl. No.: 17/607,706

(22) PCT Filed: Apr. 30, 2020
(86) PCT No.: PCT/EP2020/062097

§ 371 Date: Oct. 29, 2021

(30) Foreign Application Priority Data
Apr. 30, 2019 (EP) 19171757.8
Apr. 30, 2019 (EP) 19171765.1

(51) Int. Cl. C07K 16/28; A61K 45/06; A61K 39/395; G16B 15/30; A61K 31/47

(52) U.S. Cl. CPC C07K 16/28; A61K 45/06; A61K 39/3955; G16B 15/30; A61K 31/47; C07K 2317/569; C07K 2317/565; C07K 2317/567; C07K 2317/24; C07K 2317/31; C07B 2200/13; C07K 2317/92

(57) ABSTRACT

The present invention relates to binding agents specific for the cystic fibrosis transmembrane conductance regulator (CFTR), which increase its thermal stability to provide for potent therapeutics. More particular, the immunoglobulin single variable domains (ISVDs) identified herein reveal novel binding sites on the nucleotide-binding domain 1 of CFTR, which allow to rescue pathogenic mutant F508del CFTR from proteasomal degradation. The binding agents are therefore considered suitable in treatment of cystic fibrosis. Finally, also crystalline structures demonstrating binding interfaces, and computer-assisted methods for selecting molecules able to stabilize CFTR are described.

### CROSS-REFERENCE TO RELATED APPLICATIONS

This application is a national phase entry under 35 U.S.C. § 371 of International Patent Application PCT/EP2020/062097, filed Apr. 30, 2020, designating the United States of America and published in English as International Patent Publication WO 2020/221888 on Nov. 5, 2020, which claims the benefit under Article 8 of the Patent Cooperation Treaty to European Patent Application Serial No. 19171757.8, filed Apr. 30, 2019 and European Patent Application Serial No. 19171765.1, filed Apr. 30, 2019, the entireties of which are hereby incorporated by reference.

### FIELD OF THE INVENTION

The present invention relates to binding agents specific for the cystic fibrosis transmembrane conductance regulator (CFTR), which increase its thermal stability to provide for potent therapeutics. More particular, the immunoglobulin single variable domains (ISVDs) identified herein reveal novel binding sites on the nucleotide-binding domain 1 of CFTR, which allow to rescue pathogenic mutant F508del CFTR from proteasomal degradation. The binding agents are therefore considered suitable in treatment of cystic fibrosis. Finally, also crystal structures demonstrating binding interfaces, and computer-assisted methods for selecting molecules able to stabilize CFTR are described.

### BACKGROUND

Cystic Fibrosis (CF) is caused by a defect in the cystic fibrosis transmembrane conductance regulator (CFTR) gene, resulting in thick mucus and very salty sweat, and is one of the most common lethal genetic disease in Western Countries. CFTR is an ion channel responsible for controlling transport of chloride and carbonate across the epithelia in a number of tissues including the lungs. Although it functions as an cAMP-regulated chloride channel, CFTR belongs to the ATP-binding cassette (ABC) transporter superfamily from a structural and evolutionary standpoint. The recent electron cryo-microscopy (cryo-EM) structures of zebrafish and human CFTR^{[2-4]}, have confirmed that it adopts the common architecture of ABC proteins, with 12 transmembrane helices (TMDs) and two nucleotide-binding domains (NBDs) located in the cytoplasm. The NBDs are connected to the TMDs by short coupling helices named intracellular loops (ICLs). While crystallographic studies indicated that isolated NBDs can form well-structured domains^{[5-7]}, they appear less defined than the TMDs in the cryo-EM structures, with higher B factors which may reflect the dynamic character of these regions. Another hallmark of CFTR is its additional dynamic cytoplasmic domain, named R domain^{[8]} that controls channel gating in response to phosphorylation by protein kinase A (PKA). The R domain is only partly seen on the cryo-EM structure of dephosphorylated CFTR^{[2]} and appears to be located between the TMDs.

CFTR is expressed in several epithelia, including the sweat duct, airway, pancreatic duct, intestine, biliary tree, and vas deferens. In normal epithelial cells (such as those lining the lung), an outward flow of chloride ions from the cell is opposed by sodium reabsorption, resulting in a delicate balance of water in the lumen to maintain optimal periciliary fluid and mucus rheology. Cells with a defective CFTR exhibit excessive sodium absorption via the epithelial sodium channel which results in the build-up of viscous mucus^{[49]}. As a consequence of this thick mucus, the cystic fibrosis airway is exposed to a vicious cycle of obstruction, infection, and inflammation. Infections become chronic due to a phenotypic switch from nonmucoid to mucoid variants which are resistant to antibiotics and the innate host response^{[50]}.

Over 300 cystic fibrosis-causing mutations have been described in the CFTR gene (see https://www.cftr2.org/), and they are spread over various parts of the protein, indicating that several pathogenic mechanisms are possible. It is now recognized that the intrinsic dynamics and relative instability of the protein are central elements in the physiopathology of CF. The most dramatic illustration of this behavior is that the deletion of phenylalanine F508 (F508del) in NBD1, a residue making contact with ICL4, perturbs NBD1 thermodynamic stability, and the interface between the NBDs and the TMDs^{[10-12].} The leading cause of CF in about 90% of the patients is the F508del mutation in CFTR, leading to misfolding and early degradation of CFTR, and so to its clearance by the quality control system, which results in disruption of ionic and water homeostasis in epithelial cells of various organs such as lungs, pancreas, and intestine. This deleterious effect can be compensated by a variety of mutations in NBD1 at different locations. Introducing such stabilizing mutations in a F508del CFTR background permits maturation of a functional channel^{[5,6,13]}. Remarkably, the extent of recovery in protein maturation seems to be directly proportional to the ability of specific compensating mutations to increase thermal stability of NBD1^{[14]}. CFTR also contains a 32-residue segment termed the regulatory insertion (RI), located in position 405-436 in NBD1, not present in other ATP-binding cassette transporters. Removal of RI enables F508del CFTR to mature and traffic to the cell surface where it mediates regulated anion efflux and exhibits robust single chloride channel activity^{[9]}.

Years of research have indicated that compounds able to re-stabilize the protein would provide efficient therapeutic routes. However, currently available drugs do not appear to stabilize the mutant protein, which could explain their very limited efficiency. Indeed, current correctors of CFTR have a limited ability to improve patient's conditions and their mechanisms of action remain poorly understood. This is particularly true for correctors of the F508del mutant protein promoting CFTR maturation without restoration of its thermodynamic stability^{[15]}. In this context, developing NBD1-specific chaperones with the ability to improve the thermostability of CFTR, including the F508del mutant, would provide a promising route for effective therapy.

### SUMMARY OF THE INVENTION

The present application encompasses a new approach for CFTR stabilization for therapeutic developments. More particular, the recognized ability of nanobodies to thermally stabilize a specific conformation of their target antigen, human CFTR, via binding pockets on NBD1, opens new routes for drug discovery. Characterization of the VHH interaction with CFTR further demonstrated the ability of several of them to bind a specific site on CFTR resulting in thermal stabilization of CFTR in its wild type and/or F508del mutant form. Said thermal stabilization being acknowledged for instance by a difference in melting temperature of CFTR upon binding the VHH as an increase of 5° C. or more as compared to non-bound CFTR. The identification of several novel epitopes demonstrates that CFTR must be able to adopt conformations that differ significantly from the currently known cryo-EM structures, further establishing that CFTR is a highly dynamic protein, even under a normal physiological regime.

In a first aspect, the invention relates to a binding agent directed against the Cystic Fibrosis Transmembrane Conductance Regulator (CFTR), which increases the thermal stability of CFTR upon binding, resulting in an at least 5° C. melting temperature (Tm) increase for CFTR protein as compared to a negative control, such as an unbound CFTR under the same conditions. In one embodiment, the binding agent specifically binds the nucleotide-binding domain 1 (NBD1) of CFTR, and/or increases the melting temperature of NBD1 with at least 5° C. to a non-VHH-bound NBD1. In particular embodiments, said binding agent specifically recognizes the CFTR binding site (also referred to herein as ‘epitope 1’) comprising amino acid residues Ala457, Ser459, Gly550-Gly551, Gly576, Tyr577, Leu578, Asp579, Va1580, Leu581, Ser605, Lys606, Met607, Glu608, Leu610, Ile618, Tyr625, and Leu633 of human CFTR, as presented in SEQ ID NO:1, in particular of the human CFTR NBD1 domain. Said binding agent is also capable of binding the same binding site of the human CFTR protein carrying the F508del mutation. More specific embodiments relate to those binding agents being a small molecule compound, a chemical, a peptide, a peptidomimetic, an antibody mimetic, an immunoglobulin single variable domain (ISVD) or an active antibody fragment. Even more specifically, the binding agents comprise ISVDs. In particular, said ISVDs comprise an amino acid sequence comprising 4 framework regions (FR) and 3 complementarity determining regions (CDR) according to the following formula (1): FR1-CDR1-FR2-CDR2-FR3-CDR3-FR4 (1); and/or said ISVDs comprise a CDR1 consisting of a sequence selected from the group of SEQ ID NO: 9, 16, 23, 30; a CDR2 consisting of a sequence selected from the group of SEQ ID NO: 11, 18, 25, 32; and a CDR3 consisting of a sequence selected from the group of SEQ ID NO: 13, 20, 27, 34. Another embodiment discloses the binding agents as ISVD comprising the sequences of Nanobody™ (Nb) D12 (SEQ ID NO:2), Nb T2a (SEQ ID NO:3), NbT27 (SEQ ID NO:4), or Nb G5 (SEQ ID NO:5), or a sequence with at least 90% amino acid identity with SEQ ID NO: 2-5, or a humanized variant thereof.

In another embodiment, the binding molecule causing increased thermal stability of CFTR upon binding, resulting in an at least 5° C. melting temperature (Tm) increase for CFTR protein as compared to a negative control, such as an unbound CFTR in the same conditions, and specifically binds the CFTR binding site (also referred to herein as ‘epitope 2’) comprising amino acid residues Met472, Glu474, Phe490, Phe494, Ser495, Trp496, Ile497, Met498, Pro499, 508-510, 560, and 564 of human CFTR, as presented in SEQ ID NO:1. More specific embodiments relate to those binding agents being a small molecule compound, a chemical, a peptide, a peptidomimetic, an antibody mimetic, a immunoglobulin single variable domain (ISVD) or an active antibody fragment. Even more specifically, the binding agents comprise ISVDs. In particular said ISVDs comprise an amino acid sequence comprising 4 framework regions (FR) and 3 complementarity determining regions (CDR) according to the following formula (1): FR1-CDR1-FR2-CDR2-FR3-CDR3-FR4 (1); and/or said ISVDs comprise a CDR1 consisting of a sequence selected from the group of SEQ ID NO: 37, 44; a CDR2 consisting of a sequence selected from the group of SEQ ID NO: 39, 46; and a CDR3 consisting of a sequence selected from the group of SEQ ID NO: 41, 48. Another embodiment discloses the binding agents as ISVD comprising the sequences of Nb T4 (SEQ ID NO:6), Nb T8 (SEQ ID NO:7), or a sequence with at least 90% amino acid identity with SEQ ID NO: 2-5, or a humanized variant thereof.

In another aspect, the invention relates to a multi-specific binding agent, comprising at least one of these binding agents as referred to herein, i.e. a binding agent causing increased thermal stability of CFTR upon binding, resulting in an at least 5° C. melting temperature (Tm) increase for CFTR protein as compared to a negative control CFTR protein, such as an unbound CFTR in the same conditions, and specifically binding at least one of both binding sites, CFTR epitope 1 or epitope 2. Another embodiment discloses the multi-specific binding agent comprising at least one of said CFTR binding agents wherein said multi-specific binding agent is formed by coupling said CFTR binding agent to another binding agent, via a linker or a spacer. Said other binding agent(s) may comprise the same target protein, so CFTR, with a different binding site as compared to the first binding agent, or may relate to a binding agent for a different target protein, such as for instance a half-life extension. In another embodiment, the invention relates to a multi-specific binding agent, comprising a first binding agent according to binding to the CFTR binding site (epitope 1) comprising amino acid residues Ala457, Ser459, Gly550-Gly551, Gly576, Tyr577, Leu578, Asp579, Va1580, Leu581, Ser605, Lys606, Met607, Glu608, Leu610, Ile618, Tyr625, and Leu633 of human CFTR, and a second binding agent specifically recognizing the CFTR binding site (epitope 2) comprising amino acid residues Met472, Glu474, Phe490, Phe494, Ser495, Trp496, Ile497, Met498, Pro499, 508-510, 560, and 564 of human CFTR, as presented in SEQ ID NO:1, wherein said first and second binding agents are coupled via a linker or spacer, and optionally can be linked to further binding agents, for the same (CFTR) or different protein targets. A further embodiment relates to the multi-specific binding agent being a bispecific binding agent, wherein both binding agents specifically bind CFTR protein via a different binding site, which may for instance be the binding to epitope 1 and/or epitope 2, as defined herein. In a particular embodiment, said binding agents of the multi-specific binding agent comprise ISVDs. In particular embodiments, said binding agents comprise a combination of the ISVDs as described herein, either defined by their CDRs or defined by the SEQ ID NOs, wherein the first binding agent may comprise SEQ ID NO:2-5 and the second binding agent may comprise SEQ ID NO:6-7.

Another aspect of the invention relates to a composition comprising at least one of the CFTR binding agents as disclosed herein, or the multi-specific binding agent as disclosed herein. A further embodiment relates to a composition comprising the combination of at least one of the CFTR binding agents as described herein and at least one small molecule compound, wherein said small molecule compound is a CFTR corrector and/or a CFTR potentiator.

Another embodiment relates to a host cell or a vector for expression of the binding agent or the multi-specific binding agent according to the invention in a cell or in a subject, preferably a viral vector, lentiviral, adenoviral or adeno-associated viral vector.

Another aspect of the invention relates to the CFTR binding agent, multi-specific binding agent, the vector for expression of the binding agent, or the composition as disclosed herein, for use as a medicament. A specific embodiment of the invention relates to the binding agent or the composition as disclosed herein, for use in treatment of cystic fibrosis or CFTR-related disorders.

Another aspect of the invention relates to a complex comprising CFTR and a CFTR binding agent as described herein. Alternatively, the complex comprises the NBD1-domain of CFTR and a CFTR binding agent as described herein. In a specific embodiment, any of said complexes is in a crystalline form. More specifically, the complex comprises CFTR or NBD1 protein and a CFTR binding agent which is an ISVD, or a multi-specific binding agent comprising an ISVD, in particular an ISVD comprising the CDRs as disclosed herein, or an ISVD comprising SEQ ID NO: 2-7 or a sequence with at least 90% amino acid identity thereof, or a humanized variant thereof. In a specific embodiment said CFTR/ISVD complex is crystalline.

Another embodiment discloses a crystal composition containing the CFTR NBD1 domain, and a CFTR binding agent as described herein, wherein the NBD1 domain is a domain with an amino acid sequence corresponding to the 2PT-NBD1 domain (see Examples; SEQ ID NO:58) or to the ΔRI NBD1 domain (see Examples; SEQ ID NO:59) or a domain corresponding to a sequence with at least 90% identity to SEQ ID NO:58 or SEQ ID NO:59, and is further characterized in that the crystal is:
i) a crystal between the NBD1 domain of CFTR and said binding agent in the space group C121, with the following crystal lattice constants: a=152.2 Å±5%, b=41.6 Å±5%, c=99.3 Å±5%, α=90°, β=120.56°, γ=90°, or
ii) a crystal between the NBD1 domain of CFTR and said binding agent in the space group C222₁, with the following crystal lattice constants: a=38.68 Å±5%, b=135.78 Å±5%, c=190.65 Å±5%, α=β=γ=90°, or
iii) a crystal between the NBD1 domain of CFTR, and said binding agent in the space group P2₁2₁2₁, with the following crystal lattice constants: a=64.49 Å±5%, b=118.15 Å±5%, c=180.21 Å±5%, α=β=γ=90°, or
iv) a crystal between the NBD1 domain of CFTR, and said binding agent in the space group P12₁1, with the following crystal lattice constants: a=80.94 Å±5%, b=55.19 Å±5%, c=114.99 Å±5%, α=90°, β=103.96°, γ=90°.

Another embodiment relates to said crystal as described above, which has a three-dimensional structure wherein said crystal i) comprises an atomic structure characterized by the coordinates of PDB: 6GJS or a subset of atomic coordinates of PDB: 6GJS, or wherein the crystal ii) comprises an atomic structure characterized by the coordinates of PDB: 6GJU or a subset of atomic coordinates of PDB: 6GJU, or wherein the crystal iii) comprises an atomic structure characterized by the coordinates of PDB: 6GJQ or a subset of atomic coordinates of PDB: 6GJQ, or wherein the crystal iv) comprises an atomic structure characterized by the coordinates of PDB: 6GK4 or a subset of atomic coordinates of PDB: 6GK4.

Another embodiment describes the binding site of said binding agent to said NBD1 domain, consisting of a subset of atomic coordinates, present in the crystals i), ii), iii) or iv) as defined herein, wherein said binding site consists of the binding site of the T2a or D12 Nbs, namely the binding site (epitope 1′) corresponding to residues 457, 459, 550-551, 576-581, 605-608, 610, 618, 625, 633 and 636 of CFTR (SEQ ID NO:1), or the binding site of the T27 Nb, namely the site (epitope 1″) corresponding to residues 457-460, 550-551, 576-581, 605-608, 610, 618, 620, 625, and 633 of CFTR (SEQ ID NO:1), or the binding site of the T4 Nb, namely the site (epitope 2′) corresponding to residues 469, 472, 474, 488-490, 494-499, 508-510, 553, 560, and 564 of CFTR (SEQ ID NO:1), or the binding site of the T8 Nb, namely the site (epitope 2″) corresponding to residues 472, 474, 490, 492, 494-499, 504, 506, 508-510, 560, and 564, of CFTR (SEQ ID NO:1), wherein said amino acid residues represent the binding agent's CFTR NBD1 binding site. The binding sites epitope 1′ and epitope 1″ contain the minimal epitope residues of epitope 1, and all together, said minimal epitope 1 on NBD1 is bound by said Nbs capable of stabilizing wild type as well as F508del mutant CFTR proteins. Similarly, the binding sites epitope 2′ and epitope 2″ contain the minimal epitope residues of epitope 2, and all together, said minimal epitope 2 on NBD1 is bound by said Nbs capable of stabilizing at least wild type CFTR protein. Moreover, said stabilizing effect of said stabilizing NBs as used herein refers to an increase of more than 5° C. in melting temperature CFTR protein when bound, a newly technical effect that has never been observed for any CFTR binding agents.

In a final aspect of the invention, a computer-assisted method of identifying, designing or screening for a modulator of CFTR is described, wherein said modulator may be a stabilizer, which is a binding agent selected from the group consisting of a small molecule compound, a chemical, a peptide, a peptidomimetic, an antibody mimetic, an ISVD, or an active antibody fragment, and further comprises:
i) introducing into a suitable computer program parameters for defining the 3D structure of the binding site as described herein by the atomic coordinates of the corresponding crystals,
ii) creating the 3D structure of a test compound in said computer program, and
iii) displaying a superimposing model of said test compound on the 3D model of the binding site; and assessing whether said test compound model fits spatially and chemically into said binding site.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIG. 1. Binding of NBD1-specific nanobodies to isolated 2PT-NBD1 and F508del-2PT-NBD1.

(a) Nanobody binding to 2PT-NBD1 measured by ELISA. Biotinylated 2PT-NBD1 was immobilized on avidin-coated plates and incubated with increasing concentration of each nanobody. Binding of nanobody was followed by immunodetection of the His-tag (see Methods). Representative curve of 3 independent experiments is shown. Error bars represent the standard deviation (SD) of duplicates. Data were normalized to maximum signal for each nanobody separately. (b) Nanobody binding to F508del-2PT-NBD1 measured by ELISA as described in panel a. (c) Nanobody T4 binding to 2PT-NBD1 and F508del-2PT-NBD1. Data were normalized to maximum signal of T4 binding to 2PT-NBD1. (d) Nanobody T8 binding to 2PT-NBD1 and F508del-2PT-NBD1. Data were normalized to maximum signal of T8 binding to 2PT-NBD1. (e) Thermodynamic parameters of nanobody binding to 2PT-NBD1 determined using isothermal calorimetry (curves shown in FIG. 9), and pEC50 determined by ELISA (panel a). KD values determined by ITC represent mean±SD (n=3).

FIG. 2. Stabilization of isolated 2PT-NBD1 and F508del-2PT-NBD1 variants by nanobodies.

(a) Differential scanning fluorescence (DSF) of purified 2PT-NBD1. The protein (alone or in complex with one or two different nanobodies) was incubated with SYPRO Orange dye and fluorescence was measured as a function of temperature. The melting temperatures (Tm) were determined by the maxima of the first derivative of fluorescence. Curves depict mean of duplicates of one experiment representative of at least three independent experiments. (b) Summary of melting temperature differences (ΔTm) of 2PT-NBD1 in presence of different nanobodies determined using DSF as in panel (a). Data are mean±SEM of duplicates from four independent experiments. (c) Summary of melting temperature differences (ΔTm) of F508del-2PT-NBD1 in presence of different nanobodies using DSF as in panel (a). Data are mean of duplicates ±SEM (n=4).

FIG. 3. Crystal structures of NBD1-nanobody complexes.

(a) Structures of nanobodies D12, T2a and T27 bound to NBD1. Superimposition was performed on the NBD1 region. (b) Structure of NBD1-nanobody D12 complex highlighting the different structural elements of hNBD1 as well as the CDRs of the nanobody (c) Details of the interface between nanobody D12 and NBD1. Polar interactions are highlighted by dashed lines. Only side chains participating in the interface are explicitly shown. (d) Structures of nanobodies T4 and T8 bound to NBD1. Superimposition was performed on the NBD1 region. The view is rotated compared to panel a. (e) Structure of NBD1-T4 nanobody complex highlighting the different structural elements of NBD1 as well as the CDRs of the nanobody (f) Details of the interface between nanobody T4 and NBD1. Polar interactions are highlighted by dashed lines. Only side chains participating in the interface are explicitly shown. (g) Close-up of the interaction of F508 from NBD1 to residues in T4. Atoms are shown as space-filling model to highlight the contacts, occurring at Van der Waals distances. (h) Structure of NBD1-G3a nanobody complex (i) Details of the interface between nanobody G3a and NBD1. Polar interactions are highlighted by dashed lines. Only side chains participating in the interface are explicitly shown.

FIGS. 4A-4I: Binding of nanobodies to Full-Length-CFTR.

(FIG. 4A) Dose-response ELISA of interactions between wt-CFTR and nanobodies. Immobilized nanobodies (T2a, T8 and G3a) were incubated with different concentrations of purified CFTR. (FIG. 4B) Immobilized purified wt-CFTR was incubated with increasing concentrations of nanobodies. For both (FIG. 4A) and (FIG. 4B) Data were normalized to maximal response of T2a after subtraction of the signal from the negative control nanobody. Graph depicts one representative of at least three independent experiments. Error bars represent standard deviations of triplicates. (FIG. 4C) Average Bₘₐₓ of 3 independent experiments (±SEM) calculated for curves in FIG. 4B. (FIG. 4D) Flow cytometry analysis of nanobodies T2a, T8 and G3a on parental BHK-21 cells shows no difference in labelling compared to a negative control nanobody while in (FIG. 4E) increased labelling is observed for the NBD1-specific nanobodies in BHK-21 cells overexpressing wt-CFTR. Data were normalized to the number of events acquired in each condition. Graph depicts one representative of at least three independent experiments. (FIG. 4F) Average median fluorescence (fold over negative control) for each of the three representative nanobodies as illustrated in (FIG. 4D) and (FIG. 4E). Quantification of at least 3 independent experiments (±SEM). (g) Immunoblot of CFTR from solubilized BHK-21 cells pulled-down with His-tagged nanobodies, including a non-CFTR nanobody as a control. Eluted nanobodies-CFTR complexes were separated by SDS-PAGE and presence of CFTR was detected with mAb 596 antibody after immunoblotting. Arrows indicate the mature (band C) and immature (band B) forms of CFTR. Representative of at least 3 independent experiments. (FIG. 4H) Flow cytometry analysis of nanobodies T2a, T8 and G3a on BHK-21 cells expressing 2PT-F508del showing increased labelling for T2a and G3a, but not T8. (FIG. 4I) Quantification of data illustrated in (FIG. 4I). Average of 3 independent experiments (±SEM).

FIG. 5. Nanobodies reduce ATPase activity of CFTR but increase the temperature of thermal inactivation.

(a) Influence of nanobody addition on ATPase activity of wt-CFTR. Conversion of α-³²P-ATP to ADP was measured after 1 h incubation of wt-CFTR with the different nanobodies. Data from replicate determinations are represented as mean±SEM (n=3, except for ATPase activity of wt-CFTR activity with nanobodies Neg and T4 for which n=4). (b) Thermoprotection of wt-CFTR activity by nanobodies. Inactivation threshold temperatures were determined by measuring residual ATPase activity after 30 min heat challenge at various temperatures. Data from replicate determinations are represented as mean±SEM (n=3, except for wt-CFTR activity in absence of nanobody for which n=4). (c) Thermostability of stab-CFTR measured by nanoDSF. First derivative of 350 nm fluorescence as a function of temperature showing the determination of Tm of purified stab-CFTR alone (black) or in complex with nanobody T2a (dark grey). Melting curve of nanobody T2a alone is depicted in light grey. One representative experiment shown. (d) Thermostability of stab-CFTR as in (c), in complex with nanobody T4 (dark grey). Melting curve of nanobody T4 alone is depicted in light grey. One representative experiment shown. (e) Summary of melting temperatures of stab-CFTR in complex with nanobodies T2a, T4 and T8 determined by nanoDSF. Data from triplicates are represented as mean±SD (n=2).

FIG. 6. NBD1-nanobody complexes superimposed onto the structure of CFTR.

(a) The previously reported cryo-EM structure of CFTR (PDB: 5UAK) was aligned with structure of ΔRI-NBD1-G3a complex showing that the epitope is located in the periphery of CFTR. (b) Same alignment as (a) with the structure 2PT-NBD1-T2a complex, showing a compatible binding of nanobody D12 in between the NBDs. (c) Same alignment as (a) with the ΔRI-NBD1-T8 complex where the nanobody overlaps with the TMDs, indicating that binding is not compatible with this conformational state of CFTR.

FIG. 7. NBD1 must undock from the TMDs to allow binding of nanobodies T4 or T8.

Current models indicate that CFTR alternates between a state where the two NBDs are in close contact (state A), leading to channel opening, and a state where the NBDs separate leading to channel closing (state B). State A would typically be induced by PKA phosphorylation. States A and B have been observed by cryo-EM 2,4 and both bury F508 in the NBD1-TMD interface. Nanobodies T4 and T8 bind an epitope containing F508 (illustrated in purple), thus requiring a transient undocking of NBD1 from the TMDs (state C). This transient state can be stabilized upon binding of these nanobodies (state D).

FIG. 8. Multiple alignment of the selected nanobody sequences.

Amino acid sequences of D12, T2a, T27, T4, T8 and G3a nanobodies selected for this study. The complementarity-determining region (CDR) sequences alternating with framework (FR) sequences were identified according to International ImMunoGeneTics information system amino acid numbering (http://http://www.imgt.org/). The alignment has been generated using ClustalX.

FIGS. 9A-9E: Representative thermograms obtained by titrations of nanobodies T2a, T27, T4, T8 and G3a into 2PT-NBD1 at 20° C.

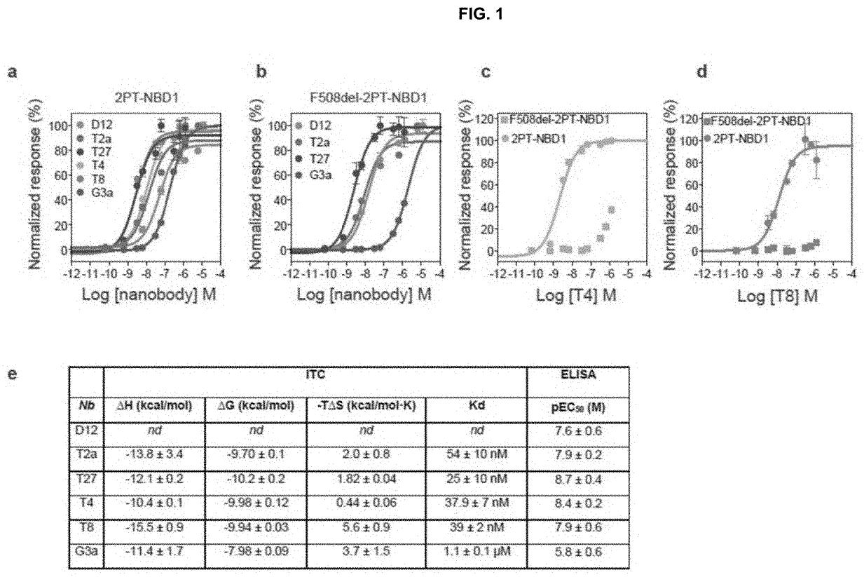

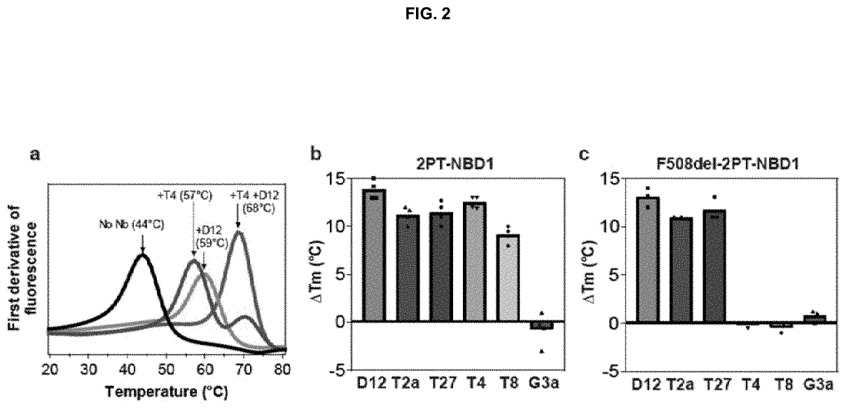

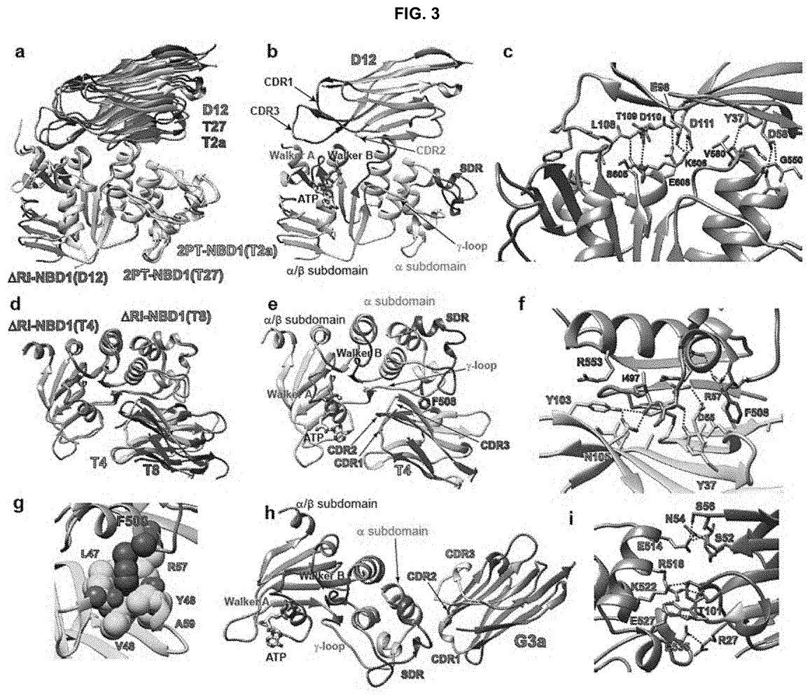

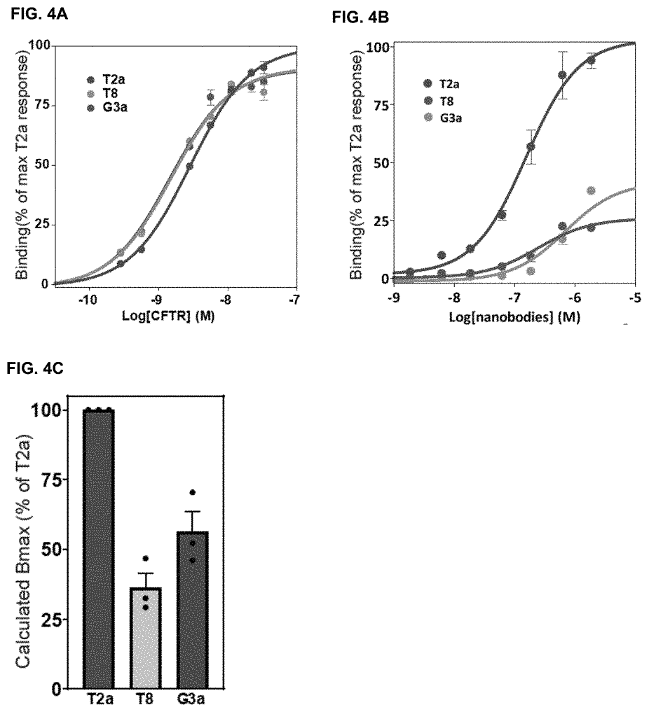

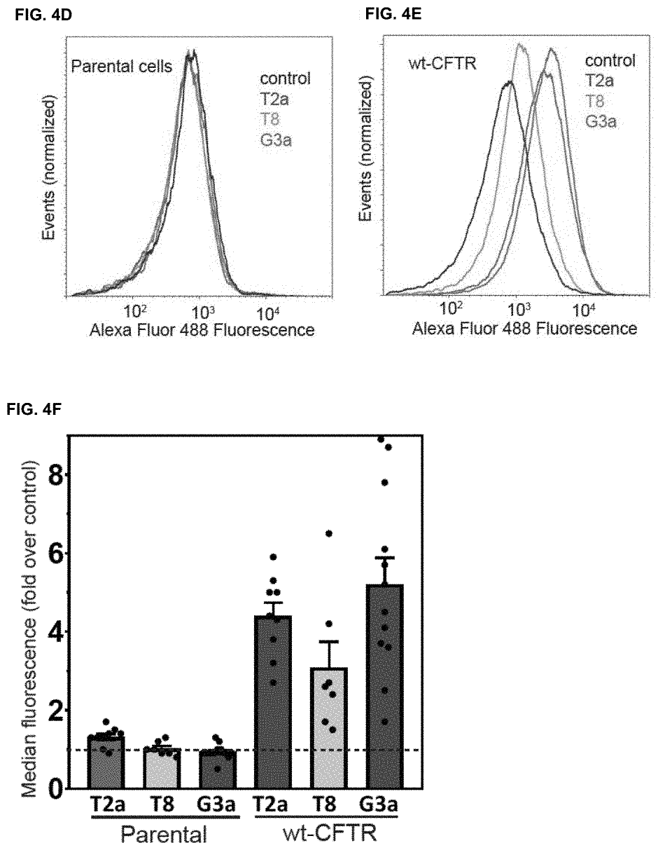

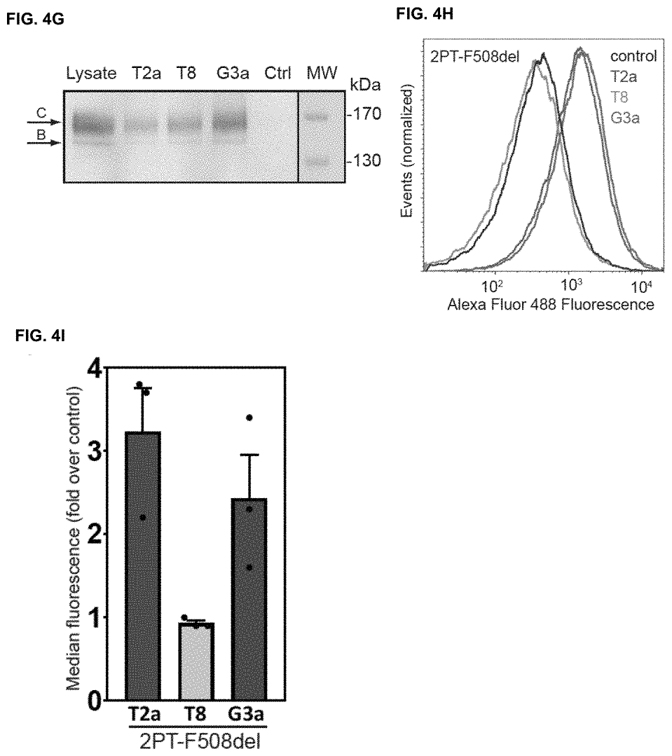

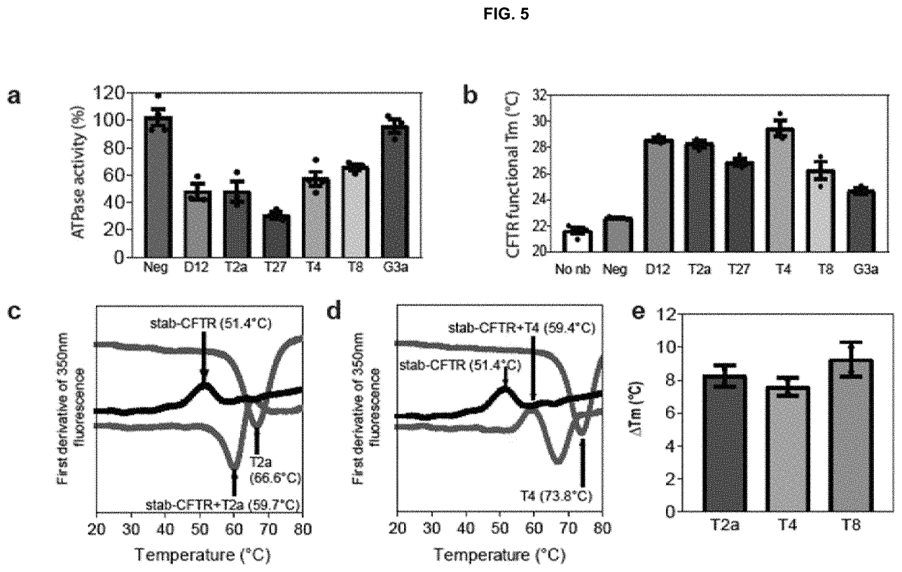

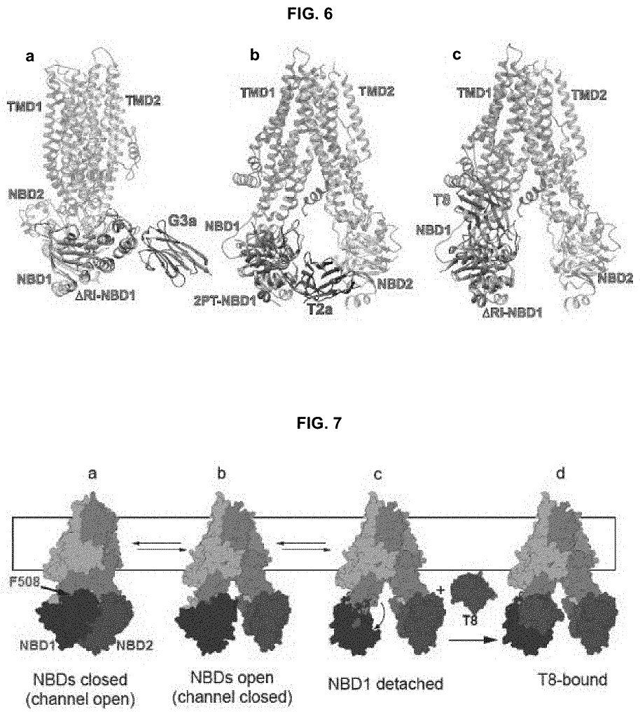

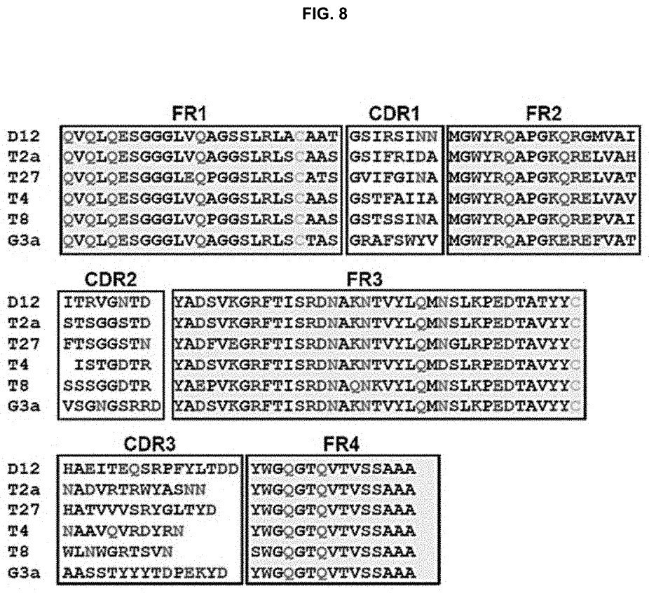

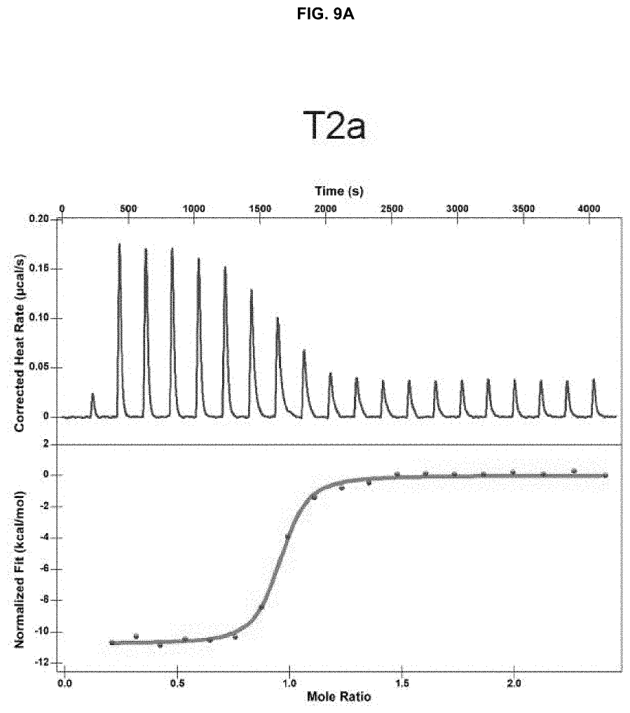

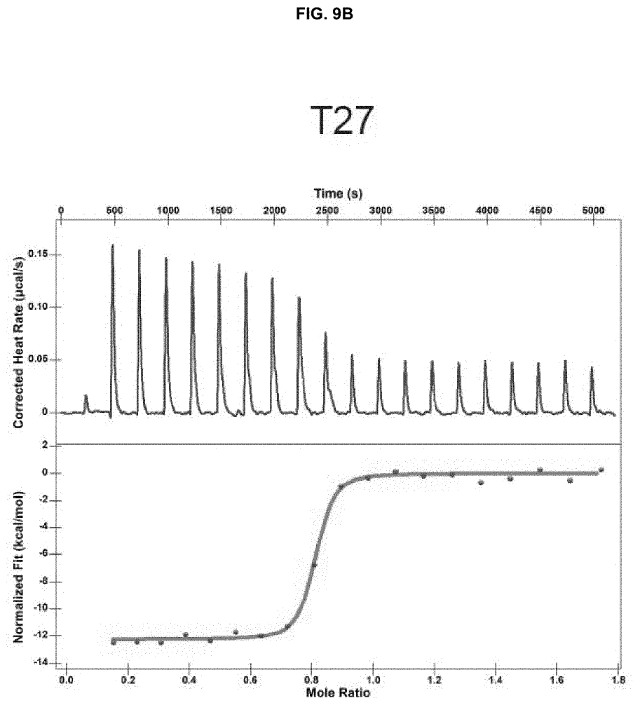

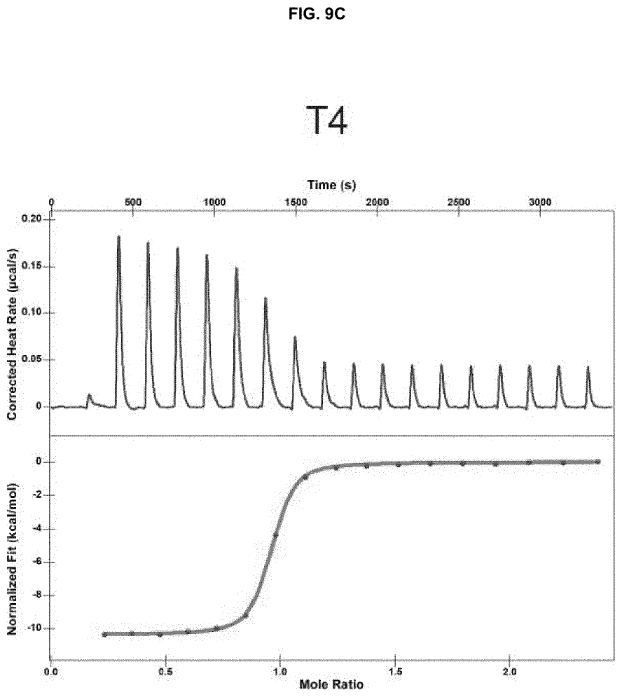

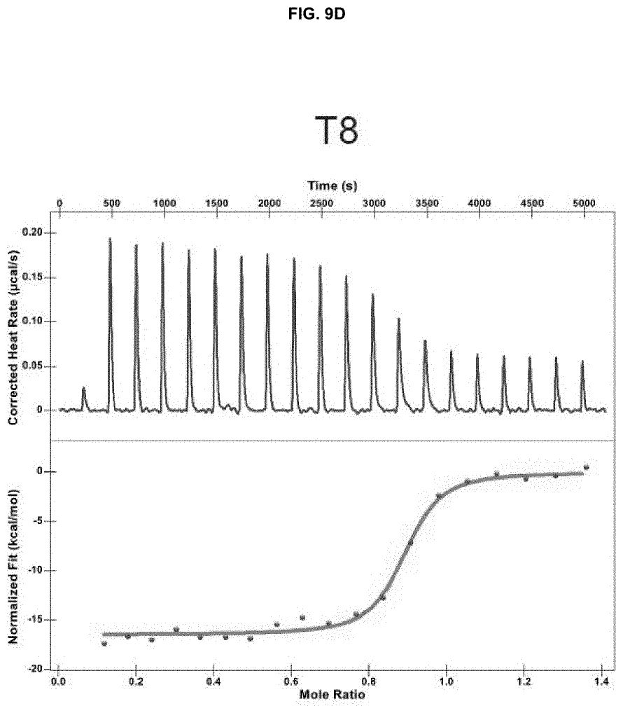

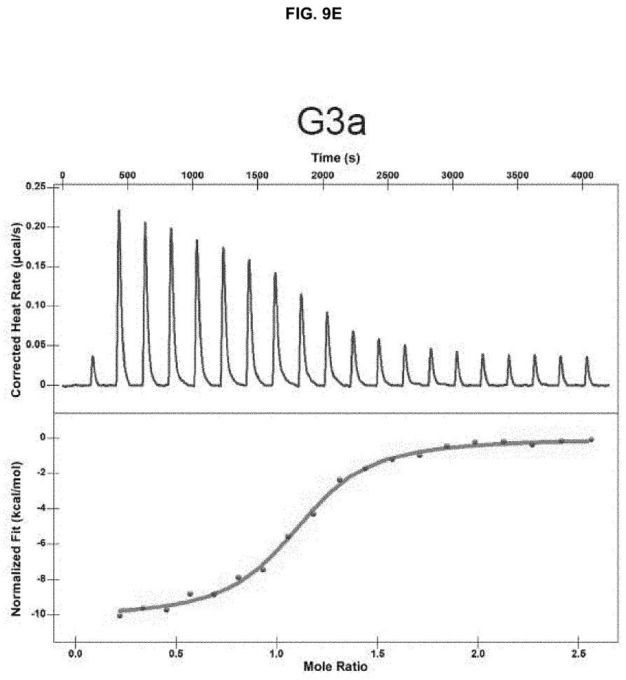

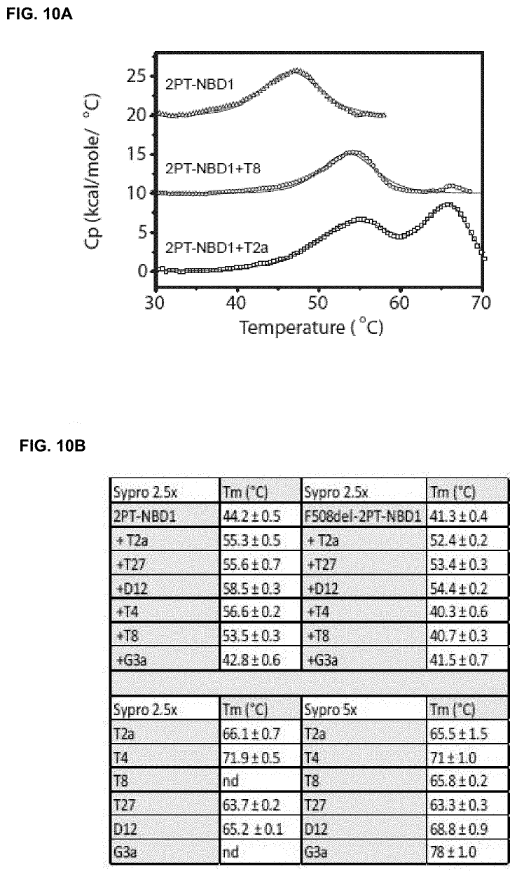

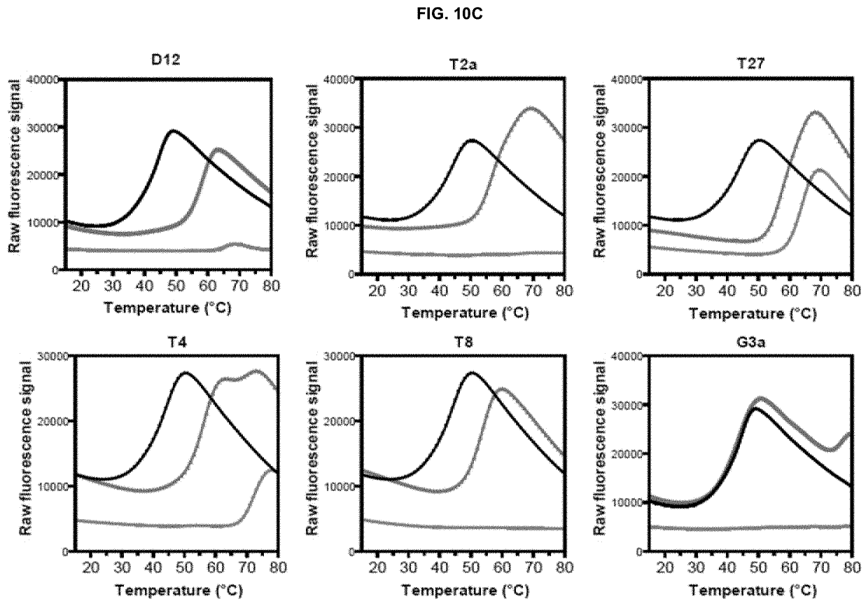

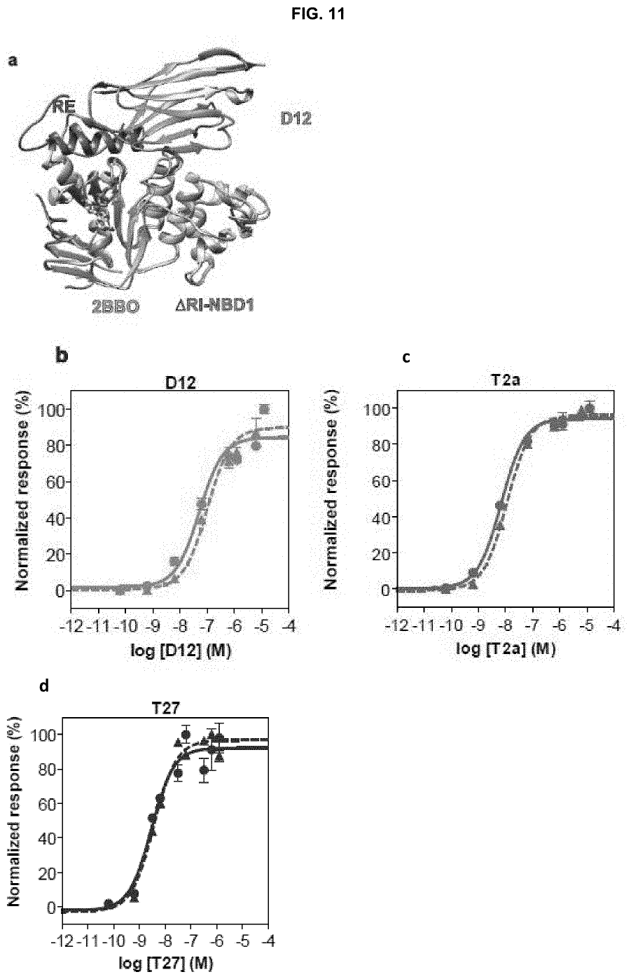

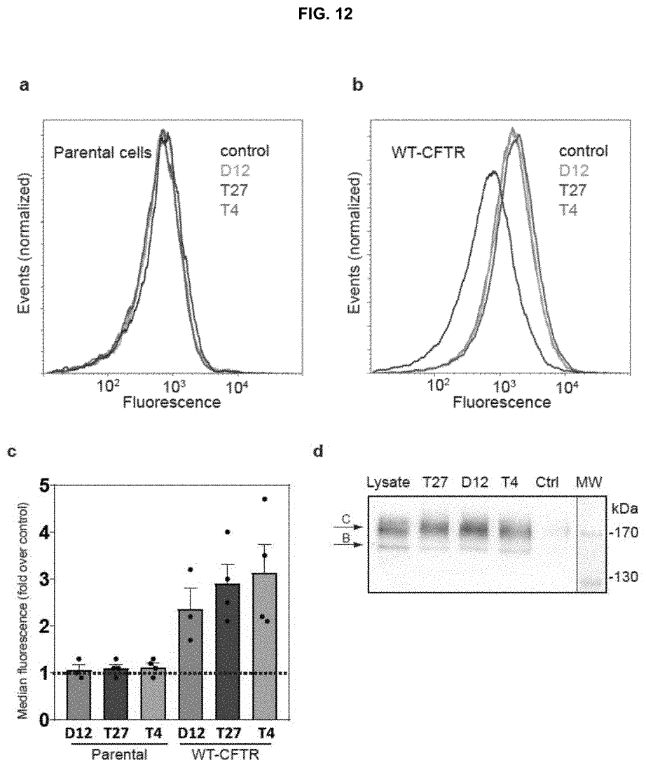

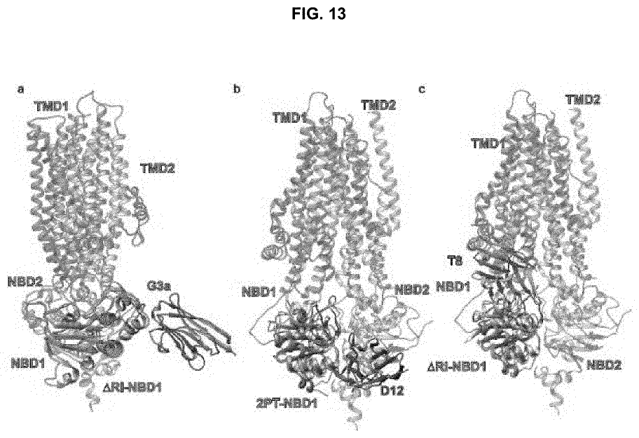

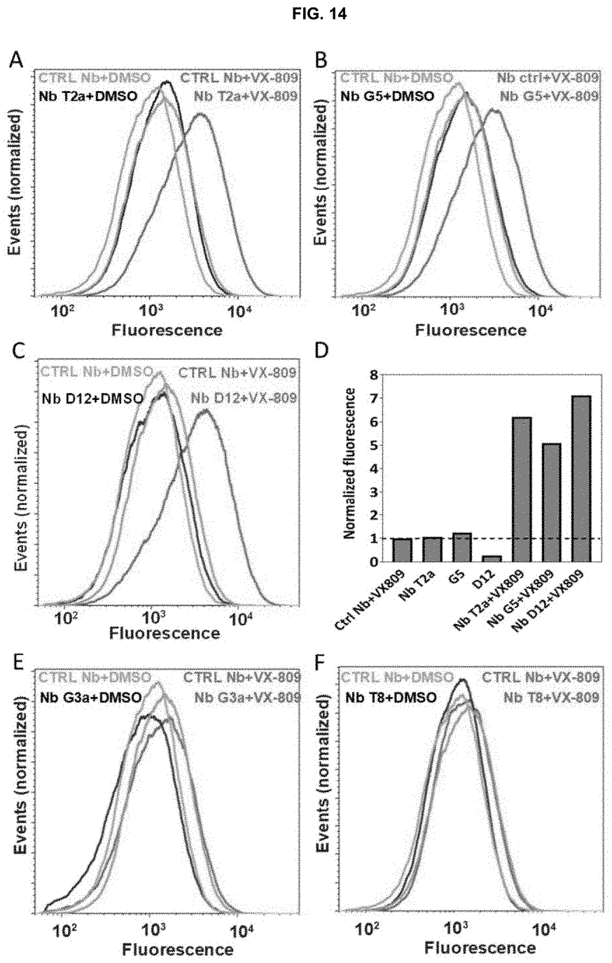

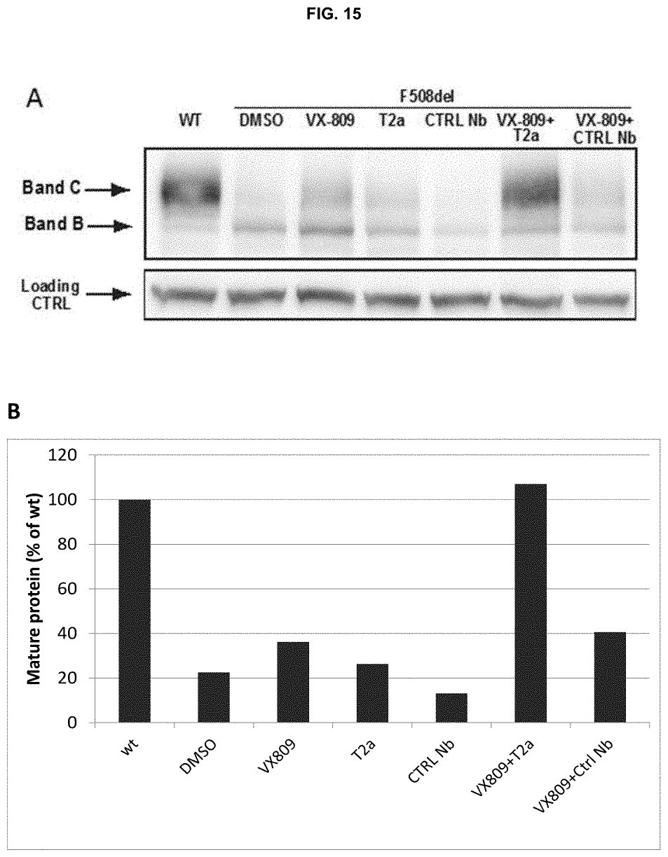

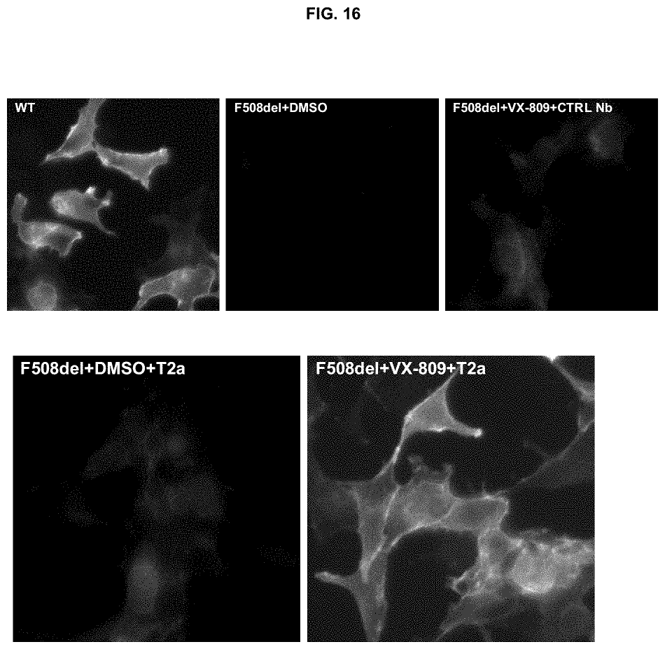

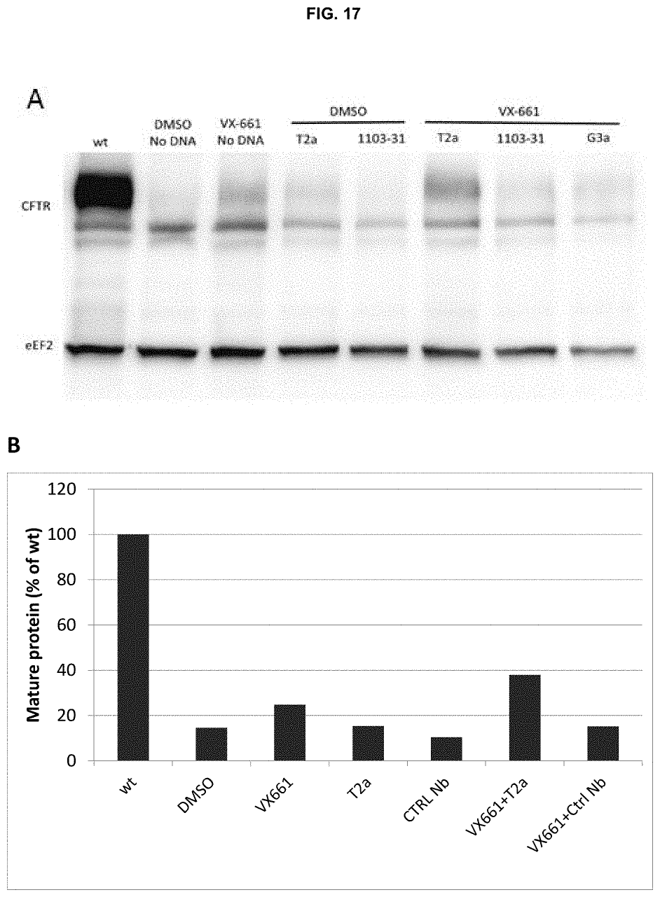

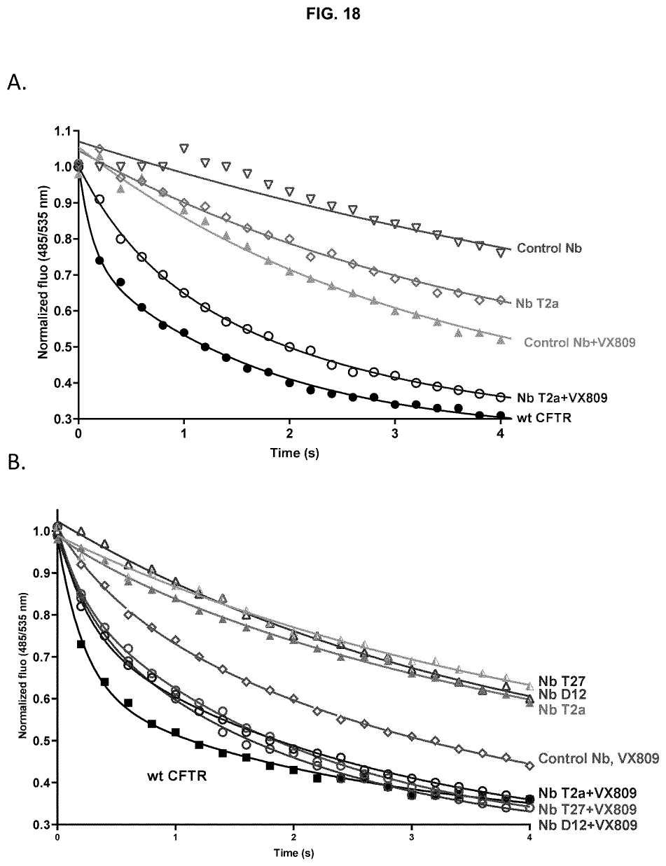

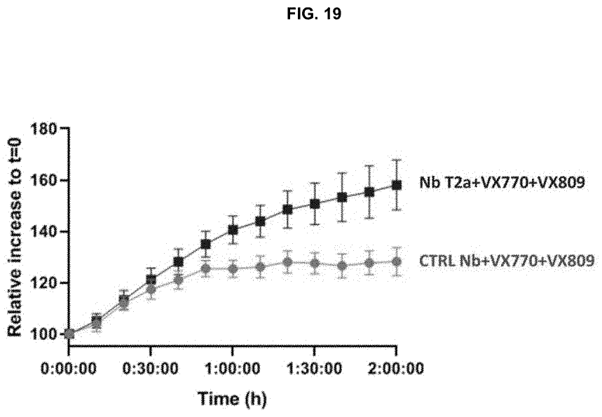

Upper panels show raw data, and lower panels represent the integration of heat changes associated with each injection of nanobodies. Data were fitted using a one-site binding model as described in Methods. Computed parameters are presented in FIG. 1. Representative curve of 3 independent experiments is shown.

FIGS. 10A-10C: Thermostabilization of NBD1 by nanobodies.

(FIG. 10A) Stacked overlay of DSC fitted curves obtained with 2PT-NBD1 alone or stabilized with nanobodies T2a

and T8. Representative curve of 2 independent experiments is shown. (FIG. 10B) Summary table of melting temperatures of 2PT-NBD1 and F508del-2PT-NBD1 in absence and/or presence of different nanobodies (top panel) determined using DSF as in panel (FIG. 10C). The lower part of the table shows melting temperatures of isolated nanobodies at 2 different concentration of Sypro-Orange dye. Data are mean±SEM of duplicates from four independent experiments. (FIG. 10C) Raw fluorescence signal of thermal unfolding scans of 2PT-NBD1 in the absence (black curves) and presence of nanobody (green curves) were acquired by DSF using 2.5× Sypro-Orange. Unfolding of nanobody alone is depicted as grey curves. Curves depict mean of duplicates of one experiment representative of three independent experiments.

FIG. 11. Regulatory Extension (RE) of NBD1 does not impede binding of D12, T2a and T27 nanobodies.

(a) Superimposition of published structure of human NBD1 (PDB: 2BBO) and the structure of ΔRI-NBD1 in complex with nanobody D12, showing overlap between the nanobody and the RE. (b-c-d) Doseresponse ELISA showing nanobodies D12, T2a, T27 binding to 2PT-NBD1-RE (dashed lines) or 2PTNBD1 (solid lines), as described in FIG. 1a. Representative curve of 3 independent experiments is shown. Error bars represent the standard deviation (SD) of duplicates. Data were normalized to maximum signal for each nanobody separately.

FIG. 12. Effect of Nbs D12, T27 and T4 on CFTR in BHK-21 cells.

(a) Flow cytometry analysis of nanobodies D12, T27 and T4 on parental BHK-21 cells show no difference in labelling compared to a negative control nanobody while in (b) increased labelling is observed for the NBD1-specific nanobodies in BHK-21 cells overexpressing wt-CFTR. Data were normalized to the number of events acquired in each condition. Graph depicts one representative of at least three independent experiments. (c) Average median fluorescence (fold over negative control) for each of the three nanobodies as illustrated in panel (a) and (b). Average of at least 3 independent experiments (±SEM). (d) Immunoblot of CFTR from solubilized BHK-21 cells pulled-down with His-tagged nanobodies. Eluted nanobodies-CFTR complexes were separated by SDS-PAGE and presence of CFTR was detected with 596 antibody after immunoblotting. Arrows indicate the mature (band C) and immature (band B) forms of CFTR.

FIG. 13. NBD1-nanobody complexes superimposed onto phosphorylated CFTR.

(a) Superimposition of the structure of zebrafish CFTR structure (PDB: 5W81) and ΔRI-NBD1 in complex with nanobody G3a structure showing that binding of G3a is also compatible with the phosphorylated state of CFTR. (b) Superposition of the 2PT-NBD1:T2a complex with the same CFTR structure shows that T2a binding is incompatible with the closing of the NBD1 observed in the ATP-bound CFTR structure. (c) Same superimposition as in (a) with the structure of ΔRI-NBD1 in complex with nanobody T8 suggesting a different conformational state for which a large motion of NBD1 is necessary to permit T8 binding.

FIG. 14. Cell-surface expression of F508del-CFTR in HEK-293T cells measured by flow cytometry.

An engineered extracellular 3HA-tag was used. Incubation of the cells with the small molecule corrector VX-809 (A-F) leads to a moderate increase in surface expression, as also observed upon transfection with stabilizing T2a (A), G5 (B), or D12 (C) nanobodies. Combining the two treatments of Nb and VX-809 (A-C; pink) shows strong recovery of cell-surface expression when using T2a, G5 or D12 stabilizing Nbs. No effect is observed when transfecting either the non-stabilizing nanobody G3a (E) or the nanobody T8 which stabilizes WT CFTR but not F508del (F). (D) shows a quantification of the normalized signal and reveals that a synergistic impact is observed in the combination treatments of A-C.

FIG. 15. CFTR protein maturation by Western Blot analysis in 3HA-F508del-CFTR in HEK-293T cells.

(A) Band B (^{˜}170 kDa) represents non-glycosylated immature CFTR while band C is fully glycosylated mature CFTR. Band C is absent for untreated F508del-CFTR but detectable after treatment with VX-809 or transfection with T2a. A strong Band C is observed upon combination of the two, with a staining intensity comparable to that of wt protein; (B) Quantification of band intensity, normalized to the intensity of the loading control band.

FIG. 16: Immunostaining of cell-surface expression of WT and F508del-expressing HEK-293T cells.

An engineered extracellular 3HA-ta was used. Moderate staining is observed in F508del-CFTR cells treated with VX-809 or transfected with T2a while a wild-type-like staining is seen when treated with both.

FIG. 17. CFTR protein maturation by Western Blot analysis in 3HA-F508del-CFTR in HEK-293T cells.

(A) Band B (^{˜}170 kDa) represent non-glycosylated immature CFTR while band C is fully glycosylated mature CFTR. Band C is absent for untreated F508del-CFTR but detectable after treatment with VX-661 or transfection with T2a or G3a. A strong Band C is observed upon combination of the two; (B) Quantification of band intensity, normalized to the intensity of the loading control band.

FIG. 18. HS-YFP quenching assay.

Normalized fluorescence signal measured in stimulated (10 μM forskolin and 3 μM VX770 potentiator) HEK293T cells stably overexpressing F508del-CFTR and HS-YFP upon treatment with (A) T2a Nb and/or 3 μM VX809 compound with a negative control Nb, versus fluorescence measured in wt CFTR-expressing cells; (B) T27, D12 or T2a Nb, or control Nb and/or VX809 versus fluorescence measured in wt CFTR-expressing cells.

FIG. 19. Forskolin induced organoid swelling assay.

Intestinal organoids homozygous for the F508del mutation were transduced with a lentiviral vector expressing T2A or control Nb and analyzed by forskolin induced swelling (FIS) 2 weeks later. 3 μM VX809 corrector was added 24 h prior to the FIS. FIS responses were measured over a period of 2 h, after stimulated with 5 μM forskolin, and 3 μM VX770 potentiator.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions. By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or nanobody as identified and disclosed herein which has been removed from the molecules present in the a sample or mixture, such as a production host, that are adjacent to said polypeptide. An isolated protein or peptide can be generated by amino acid chemical synthesis or can be generated by recombinant production or by purification from a complex sample. “Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

As used herein, the term “crystal” means a structure (such as a three-dimensional (3D) solid aggregate) in which the plane faces intersect at definite angles and in which there is a regular structure (such as an internal structure) of the constituent chemical species. The term “crystal” refers in particular to a solid physical crystal form such as an experimentally prepared crystal. The term “co-crystal” as used herein refers to a structure that consist of two or more components that form a unique crystalline structure having unique properties, wherein the components may be atoms, ions or molecules. In the context of current application, a co-crystal comprising the NBD1 domain and one of the herein described Nanobodies (Nbs) is equivalent to a crystal of the NBD1 domain in complex with one of the herein described Nbs. The term “crystallization solution” refers to a solution which promotes crystallization comprising at least one agent including a buffer, one or more salts, a precipitating agent, one or more detergents, sugars or organic compounds, lanthanide ions, a poly-ionic compound, and/or stabilizer. The terms “suitable conditions” refers to the environmental factors, such as temperature, movement, other components, and/or “buffer condition(s)” among others, wherein “buffer conditions” refers specifically to the composition of the solution in which the molecules are present. A composition includes buffered solutions and/or solutes such as pH buffering substances, water, saline, physiological salt solutions, glycerol, preservatives, etc. for which a person skilled in the art is aware of the suitability to obtain optimal assay performance. Suitable conditions as used herein could also refer to suitable binding conditions, for instance when Nbs are aimed to bind CFTR. Suitable conditions as used herein could also refer to suitable crystallization or cryo-EM conditions, which may alternatively mean suitable conditions wherein the aimed structural analysis is expected. Suitable conditions may further relate to buffer conditions in which thermal stability assays can be performed. The “same” conditions as referred to herein means to apply the same buffer, temperature, pH, osmolyte concentration salt content, etc . . . . for such comparison, for instance for determining the melting temperature of a protein or protein complex, as described herein.

The term “binding pocket” or “binding site” refers to a region of a molecule or molecular complex, that, as a result of its shape and charge, favourably associates with another chemical entity, compound, proteins, peptide, antibody or Nb. For antibody-related molecules, the term “epitope” or “conformational epitope” is also used interchangeably herein. The term “pocket” includes, but is not limited to cleft, channel or site. The NBD1 domain herein described comprises a binding pocket or binding site which include, but is not limited to a Nb binding site. The term “part of a binding pocket/site” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. A “binding agent” relates to a molecule that is capable of binding to another molecules, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others.

The term “molecular complex” or “complex” refers to a molecule associated with at least one other molecule, which may be a chemical entity. The term “associating with” refers to a condition of proximity between a chemical entity or compound, or portions thereof, and a binding pocket or binding site on a protein. The association maybe non-covalent—wherein the juxtaposition is energetically favored by hydrogen bonding or van der Waals or electrostatic interactions—or it may be covalent. The term “chemical entity” refers to chemical compounds, complexes of at least two chemical compounds, and fragments of such compounds or complexes. The chemical entity may be, for example, a ligand, a substrate, a phosphate, a nucleotide, an agonist, antagonist, inhibitor, antibody, a single domain antibody, drug, peptide, peptidomimetic, protein or compound.

The term “antibody” as used herein, refers to an immunoglobulin (Ig) molecule or a molecule comprising an immunoglobulin (Ig) domain, which specifically binds with an antigen. ‘Antibodies’ can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains, Nanobodies, domain antibodies, and single chain structures, such as a complete light chain or complete heavy chain. An additional requirement for “activity” of said fragments in the light of the present invention is that said fragments are capable of binding CFTR, and preferably increase CFTR thermal stability, more preferably rescue CFTR protein maturation.

The term “antibody”, “antibody fragment” and “active antibody fragment” as used herein refer to a protein comprising an immunoglobulin domain or an antigen binding domain capable of specifically binding CFTR. The antibodies or active antibody fragments of the invention may be coupled to a functional moiety, or to a cell penetrant carrier. Antibodies are typically tetramers of immunoglobulin molecules. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention also refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies and Nanoclone® are registered trademarks of Ablynx N.V. (a Sanofi Company). For a general description of Nanobodies (Nbs), reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent constructs (including some non-limiting examples of linker sequences) and different modifications to increase the half-life of the Nanobody and their preparations can be found e.g. in WO 08/101985 and WO 08/142164. Nanobodies form the smallest antigen binding fragment that completely retains the binding affinity and specificity of a full-length antibody. Nbs possess exceptionally long complementarity-determining region 3 (CDR3) loops and a convex paratope, which allow them to penetrate into hidden cavities of target antigens. In particular, humanized immunoglobulin single variable domains, such as Nanobody (including VHH domains) may be immunoglobulin single variable domains that are as generally defined for in the previous paragraphs, but in which at least one amino acid residue is present (and in particular, at least one framework residue) that is and/or that corresponds to a humanizing substitution (as defined herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody (including VHH domains) may be partially humanized or fully humanized.

An “epitope”, as used herein, refers to an antigenic determinant of a polypeptide, constituting a binding site or binding pocket on a target molecule, such as CFTR NBD1. An epitope could comprise 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure.

Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

As used herein, a “therapeutically active agent” means any molecule that has or may have a therapeutic effect (i.e. curative or stabilizing effect) in the context of treatment of a disease (as described further herein). Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent. Even more preferably, a therapeutically active agent has a curative effect on the disease. The binding agent or the composition, or pharmaceutical composition of the invention may act as a therapeutically active agent, when beneficial in treating cystic fibrosis-related diseases. The binding agent may include the CFTR binder and may contain or be coupled to additional functional groups, advantageous when administrated to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, Pa. (1980). Such functional groups may for example be linked directly (for example covalently) to the ISVD or active antibody fragment, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in a immunoglobulin single variable domain of the invention, a immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment of the invention, all using techniques of protein engineering known per se to the skilled person. Another, usually less preferred modification comprises N-linked or O-linked glycosylation, usually as part of co-translational and/or post-translational modification, depending on the host cell used for expressing the antibody or active antibody fragment. Another technique for increasing the half-life of a binding domain may comprise the engineering into bifunctional or bispecific domains (for example, one ISVD or active antibody fragment against the target CFTR and one against a serum protein such as albumin or Surfactant Protein A (SpA)—which is a surface protein abundantly present in the lungs aiding in prolonging half-life)) or into fusions of antibody fragments, in particular immunoglobulin single variable domains, with peptides (for example, a peptide against a serum protein such as albumin).

The term “compound” or “test compound” or “candidate compound” or “drug candidate compound” as used herein describes any molecule, either naturally occurring or synthetic that is designed, identified, screened for, or generated and may be tested in an assay, such as a screening assay or drug discovery assay, or specifically in the method for identifying a compound capable of modulating CFTR activity. As such, these compounds comprise organic and inorganic compounds. For high-throughput purposes, test compound libraries may be used, such as combinatorial or randomized libraries that provide a sufficient range of diversity. Examples include, but are not limited to, natural compound libraries, allosteric compound libraries, peptide libraries, antibody fragment libraries, synthetic compound libraries, fragment-based libraries, phage-display libraries, and the like. Such compounds may also be referred to as binding agents; as referred to herein, these may be “small molecules”, which refers to a low molecular weight (e.g., <900 Da or <500 Da) organic compound. The compounds or binding agents also include chemicals, polynucleotides, lipids or hormone analogs that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody mimetics, antibody fragments or antibody conjugates.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product.

As used herein, the terms “determining,” “measuring,” “assessing,”, “identifying”, “screening”, and “assaying” are used interchangeably and include both quantitative and qualitative determinations. “Similar” as used herein, is interchangeable for alike, analogous, comparable, corresponding, and -like or alike, and is meant to have the same or common characteristics, and/or in a quantifiable manner to show comparable results i.e. with a variation of maximum 20%, 10%, more preferably 5%, or even more preferably 1%, or less.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present.

The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

### DETAILED DESCRIPTION

While remarkable progress has been made in the development of CFTR corrector small molecules in the last few years, little or no mechanistic insights are available to rationalize their mode of action. On the other hand, it is known that the destabilizing effect of the F508del mutation on CFTR can be compensated by artificially introduced specific mutations at various sites in NBD1, leading to significant recovery of channel expression and activity^{[19,29]}. This shows that the molecular stress caused by the F508del mutation in CFTR can be counteracted allosterically and the development of NBD1 chaperones remains an underexplored therapeutic route. So far, few molecules have been shown to specifically stabilize CFTR or even NBD1^{[14]}. Studies have shown that small compounds such as BIA or BEIA are able to slightly stabilize the protein (<3° C. increase in Tm) but only at very high concentrations (close to mM)^{[14]}, thus precluding any therapeutic developments^{[26]}. The invention as described herein is based on the detailed analysis of crystal structures of CFTR NBD1-nanobody complexes which provide an atomic description of their novel and unique binding epitopes and reveal the molecular basis for thermal stabilization of CFTR, caused by the specific binding of these Nbs. Furthermore, novel conformational dynamics of CFTR are disclosed herein, involving detachment of NBD1 from the transmembrane domain, which contrasts with the compact assembly observed in cryo-EM structures. These unexpected dynamics are likely to have major relevance for CF pathogenesis as well as for the normal function of CFTR and other ABC proteins. So these findings as presented herein resulted in structural and functional information for a panel of different families of binding agents, in particular ISVDs, of which at least 4 Nbs bind to the same binding site of NBD1, a binding site present in wild type CFTR as well as in the pathogenic F508del mutant. These NBD1-specific CFTR stabilizing nanobodies with the ability to improve the thermostability of F508del mutant, shown as an increase in its melting temperature with at least 5° C. when bound to said Nbs as compared to non-bound CFTR, are described herein as a starting point for structure-based drug design (or intracellular delivery of biologicals). Another at least 2 Nb families were found to also stabilize wild type CFTR, by binding a second epitope (involving F508), allowing specific interaction with and stabilization of wild type CFTR, but not the mutant F508del CFTR. All these Nbs are capable to increase thermal stability of CFTR in the sense that the melting temperature of CFTR is at least 5° C. higher as compared to CFTR that is not bound to a Nb, under the same testing conditions, or CFTR bound to a non-stabilizing Nb, i.e. a control Nb. Such a high increase in Tm has never been reported as a property for any CFTR binding agent, at least not as an increase with a significant impact on mutant protein maturation. For both epitopes characterized herein, the contact residues involving the binding site are far apart from each other, thereby reducing conformational flexibility in the NBD domain when bound to the Nb. Additional data transfecting or intracellularly expressing those Nbs in CFTR-expressing cells appear to increase cell-surface expression of mutant F508del CFTR, indicative of a protective effect of the Nbs on the (mutant) channel and which suggests a stabilization of its functional conformation. Indeed, functional assays showed that the presence of stabilizing Nbs as described herein in the cells expressing F508del-CFTR, allowed an increased functionality of the mutantchannel. Thereby confirming that channel activity is not prohibited by Nbs binding the NBD1 domain of CFTR or mutant CFTR, and the stabilizing effect of the bound Nb aiding in increasing the amounts of cell-surface delivered functional CFTR. Moreover, the ‘epitope 1’ binding Nbs additionally have therapeutic potential when used in combination with current state of the art small molecule CFTR correctors since a composition applying the combination revealed a synergistic effect on maturation of the protein. Altogether, novel binding sites on CFTR were characterized, revealing to have, upon binding of CFTR binding agent, in particular of the corresponding specifically binding Nb, a thermal stabilization effect on the CFTR protein, and thereby contributing to a therapeutic potential in CFTR functionality to provide for novel insights in development of next-generation CF therapeutics for treatment of cystic fibrosis and CFTR-related disorders.

In a first aspect of the invention, a binding agent is disclosed, which specifically interacts with CFTR, more specifically via a binding site on the CFTR NBD1 domain, and resulting in increased thermostability of the CFTR protein and/or NBD1 domain as compared to the unbound CFTR or NBD1 domain. Said increase in stability involves an at least 5° C.; at least 6° C., at least 7° C., at least 8° C., at least 9° C., or at least 10° C. increase in melting temperature of CFTR or NBD1 domain when bound to said binding agent, and in comparison to a non-bound CFTR or NBD1 protein tested in the same conditions, or as compared to a CFTR or NBD1 protein bound to a non-stabilizing Nb. Examples of non-stabilizing Nbs or agents are described herein, and used as negative control. For example, the G3a Nb, which was shown to bind a different binding site of NBD1 does not have such a stabilizing effect on CFTR and is thus a non-stabilizing Nb used as vehicle control herein. Said binding agents further are specifically binding the binding site comprising amino acid residues 457, 459, 550-551, 576-581, 605-608, 610, 618, 625, and 633 of the α/β core region of NBD1 of the human CFTR as set forth in SEQ ID NO:1. Said binding site or epitope is also referred to herein as ‘epitope 1’ of the invention. Moreover, the epitope here refers to residues in human CFTR (https://www.uniprot.org/uniprot/P13569; SEQ ID NO:1) which are ‘in contact’ with the binding agent. In particular, where the epitope is described as disclosed herein ‘contact’ is defined herein as closer than 4 Å from any residue (or atom) belonging to the nanobody or binding agent of interest upon binding of a nanobody to CFTR. The binding site as defined herein is present in wild type (WT) CFTR, and in F508del mutant CTR. So the binding agents described in this embodiment are defined as stabilizing Nbs of at least wt CFTR protein and F508del CFTR. In a preferred embodiment, a binding agent is disclosed which specifically binds the CFTR NBD1 domain, thereby increasing the thermal stability of the NBD1 domain, as an increase in its melting temperature of at least 10° C. as compared to the unbound NBD1 domain. In another embodiment, the binding agent elevates the thermostability as an increase in its melting temperature of at least 8° C. of the CFTR full length as compared to the unbound CFTR protein. With thermal stability or thermostability is meant that the melting temperature of the protein is increased, and so the higher this value is for CFTR protein or NBD1 domain, the higher its activity is retained upon increasing temperature, so the higher the temperature may be to present a properly folder ion channel protein, and act as a ion channel in the membrane. The methods to measure the melting temperature as an indicator for thermostability are known to the skilled person, and are described for example herein in the methods and example section by applying a thermal shift assay (DSF) or NanoDSF. As a relative comparison, the control or vehicle sample should be the same NBD1 domain protein or CFTR protein but in absence of binding agent, and sampled in the same conditions (such as buffer, temperature, pH, etc., as described elsewhere herein). Another control or vehicle sample may comprise the same NBD1 domain protein or CFTR protein bound to a binding agent known to be non-stabilizing or negative control.

Another embodiment refers to said binding agents which are capable of increasing the thermostability of CFTR as defined herein, by specifically binding the binding site comprising amino acid residues 472, 474, 490, 494-499, 508-510, 560, and 564 of the Q-loop of NBD1 of the human CFTR as set forth in SEQ ID NO:1. Said binding site or epitope is also referred to herein as ‘epitope 2’ of the invention. The binding site as defined herein is present in wild type (WT) CFTR, and may exist in mutant CFTR, though not in F508del mutant CTR. So the binding agents described in this embodiment are defined as stabilizing Nbs of at least wt CFTR protein, but non-binding and non-stabilizing for the F508del CFTR.

In particular embodiments, said binding agent stabilizing CFTR by binding epitope 1 or epitope 2 of the invention, may be small molecule compounds, chemicals, peptides, peptidomimetics, antibody mimetics, immunoglobulin single variable domains (ISVDs) or an antibody derivative such as an active antibody fragment.

In one embodiment, the binding agents binding to epitope 1 or epitope 2, as presented herein, are ISVDs comprising at least the structure including 4 Framework regions and 3 CDRs according to the sequence of FR1-CDR1-FR2-CDR2-FR3-CDR3-FR4. Moreover, specific embodiments are provided herein with those ISVDs of the invention binding epitope 1, wherein CDR1 contains SEQ ID NO: 9, 16, 23, or 30; wherein CDR2 contains SEQ ID NO: 11, 18, 25, or 32; wherein CDR3 contains SEQ ID NO: 13, 20, 27, or 34. A further embodiment discloses those ISVDs of the invention binding epitope 1, depicted in SEQ ID NO:2-5, or depicting a sequence with at least 99%, at least 95%, at least 90%, or at least 85% identity thereof. Alternatively, said ISVDS comprise a humanized variant of SEQ ID NO: 2, 3, 4 or 5. In further specific embodiments, those ISVDs of the invention binding epitope 2 are provided, wherein CDR1 contains SEQ ID NO: 37 or 44; wherein CDR2 contains SEQ ID NO: 39, or 46; wherein CDR3 contains SEQ ID NO: 41, or 48. A further embodiment discloses those ISVDs of the invention binding epitope 2, depicted in SEQ ID NO:6 or in SEQ ID NO:7, or depicting a sequence with at least 99%, at least 95%, at least 90%, or at least 85% identity thereof. Alternatively, said ISVDS comprise a humanized variant of SEQ ID NO: 6 or 7.

The CDR region annotation for each ISVD sequence described herein is shown in FIG. 8 from the current analysis, corresponding to IMGT annotation (LeFranc, 2014; Frontiers in Immunology. 5 (22): 1-22). Alternatively, slightly different CDR annotations known in the art may be applied here and relate to the AbM (AbM is Oxford Molecular Ltd.'s antibody modelling package as described on http://www.bioinf.org.uk/abs/index.html), Chothia (Chothia and Lesk, 1987; J Mol Biol. 196:901-17), or Kabat (Kabat et al., 1991; Sequences of Proteins of Immunological Interest. 5th edition, NIH publication 91-3242), which are all applicable to identify the CDR regions of the ISVDs as disclosed herein for SEQ ID NO: 2-7. It should be noted that—as is well known in the art for VH domains and for VHH domains—the total number of amino acid residues in each of the CDRs may vary and may not correspond to the total number of amino acid residues indicated by the annotation used (that is, one or more positions according to a certain annotation may not be occupied in the actual sequence, or the actual sequence may contain more amino acid residues than the number allowed for by the annotation). This means that, generally, the numbering when using for instance the annotation according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

In another embodiment, the CFTR binding agent comprises an ISVD comprising the amino acid sequence selected from the group consisting of SEQ ID NO:2-7, or an ISVD comprising the amino acid sequence selected from the group consisting of a sequence with at least 85% identity to any of the sequences of SEQ ID NO:2-7, wherein the CDRs are identical to the CDRs of SEQ ID NO:2-7, with any annotation used possible, and differences may be present in Framework residues. In a specific embodiment, the CFTR binding agent comprises an ISVD comprising the amino acid sequence selected from the group consisting of a sequence with at least 90% identity to any of the sequences of SEQ ID NO:2-7, wherein the CDRs are identical to the CDRs of SEQ ID NO:2-7, and differences may be present in Framework residues, except for the llama germline hallmark residues present in said Framework regions. More specifically, the latter correspond to residues 37 (Kabat N°; Y in D12, T2a and T27), residue 44-45 (Kabat N°; QR in D12, T2a and T27), residue 47 (Kabat N°; M or L in D12, T2a and T27), residue 78 (Kabat N°; V in D12, T2a and T27) and residue 84 (Kabat N°; P in D12, T2a and T27), and residue 93 (Kabat N°; H or N in D12, T2a and T27), and residue 94 (Kabat N°; A in D12, T2a and T27). In another embodiment, said CFTR binding agent comprises and ISVD comprising the amino acid sequence selected from the group consisting of a humanized variant of any of the sequences of SEQ ID NO:2-7, or a humanized variant of any of the sequences with 85-95% identity to SEQ ID NO:2-7, wherein the CDRs are identical to the CDRs of SEQ ID NO:2-7, and differences may be present in the FR regions.

The term ‘humanized variant’ of an immunoglobulin single variable domain such as a domain antibody and Nanobody® (including VHH domain) refers to an amino acid sequence of said ISVD representing the outcome of being subjected to humanization, i.e. to increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other or further suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized. Humanized immunoglobulin single variable domains, in particular Nanobody, may have several advantages, such as a reduced immunogenicity, compared to the corresponding naturally occurring VHH domains. In summary, the humanizing substitutions should be chosen such that the resulting humanized amino acid sequence of the ISVD and/or VHH still retains the favourable properties, such as the antigen-binding capacity, and allosteric modulation capacity. The skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also, a subset of human germline alleles most homologous to the target sequence may be aligned as starting point to identify suitable humanisation residues. Alternatively, the VHH is analyzed to identify its closest homologue in the human alleles, and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues (i.e. hallmark residues) in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization. Suitable mutations, in particular substitutions, can be introduced during humanization to generate a polypeptide with reduced binding to pre-existing antibodies (reference is made for example to WO 2012/175741 and WO2015/173325), for example in at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined herein) or preferably at one or more other framework residues (i.e. non-Hallmark residues) or any suitable combination thereof. Depending on the host organism used to express the amino acid sequence, ISVD, VHH or polypeptide of the invention, such deletions and/or substitutions may also be designed in such a way that one or more sites for posttranslational modification (such as one or more glycosylation sites at asparagine to be replaced with G, A, or 5; and/or Methionine oxidation sites) are removed, as will be within the ability of the person skilled in the art. Alternatively, substitutions or insertions may be designed so as to introduce one or more sites for attachment of functional groups, for example to allow site-specific pegylation. In some cases, at least one of the typical Camelidae hallmark residues with hydrophilic characteristics at position 37, 44, 45 and/or 47 is replaced (Kabat N°; see WO2008/020079 Table A-03). Another example of humanization applicable to the ISVDs as described herein relates to the substitution of residues in FR 1, such as position 1, 5, and 14; in FR3, such as positions 74, and 83; and in FR4, such as position 108 (all numbering according to the Kabat). In one embodiment said humanized variant includes at least one substitution in any one of the ISVDs comprising SEQ ID NO:2-7 selected from the group of substitutions at the following positions (according to Kabat N°): residue 1 substitution to E or D; residue 14 to P; 62 to 5; 64 to K; 74 to A; 83 to R; and/or 108 to L. More preferably, said humanized variant includes at least one substitution in any one of the ISVDs comprising SEQ ID NO:2-7 selected from the group of substitutions at the following positions (according to Kabat N°): residue 1 substitution to E or D; residue 14 to P; 74 to A; 83 to R; and/or 108 to L.

Another aspect described herein concerns multi-specific binding agents comprising at least one CFTR binding agent as described herein. The nature and structure of the multispecific binding agent may be diverse, as it may be a protein coupled to a chemical moiety, or several proteins coupled to each other as well as a covalent complex of proteins with different binding specificity. The multi-specific binding agent may further comprise binding agents specific for other targets, such as for albumin or surfactant protein A to increase the half-life to the binding agent in a subject. In a particular embodiment, the multi-specific binding agent comprises immunoglobulin single variable domains of the invention, which may be present in a “multivalent” form and are formed by bonding, chemically or by recombinant DNA techniques, together two or more monovalent immunoglobulin single variable domains. Non-limiting examples of multivalent constructs include “bivalent” constructs, “trivalent” constructs, “tetravalent” constructs, and so on. The immunoglobulin single variable domains comprised within a multivalent construct may be identical or different. In another particular embodiment, the immunoglobulin single variable domains of the invention are in a “multi-specific” form and are formed by bonding together two or more immunoglobulin single variable domains, of which at least one with a different specificity. Non-limiting examples of multi-specific constructs include “bi-specific” constructs, “tri-specific” constructs, “tetra-specific” constructs, and so on. To illustrate this further, any multivalent or multi-specific (as defined herein) ISVD of the invention may be suitably directed against two or more different epitopes on the same antigen, for example against epitope 1 and epitope 2 of CFTR NBD1; or may be directed against two or more different antigens, for example against CFTR and one as a half-life extension against Serum Albumin or SpA. Multivalent or multi-specific ISVDs of the invention may also have (or be engineered and/or selected for) increased avidity and/or improved selectivity for the desired CFTR interaction, and/or for any other desired property or combination of desired properties that may be obtained by the use of such multivalent or multi-specific immunoglobulin single variable domains. For instance, the combination of one or more ISVDs binding epitope 1, and one or more ISVDs binding epitope 2 as described herein, results in a multi-specific binding agent of the invention. Said multi-specific binding agent comprises at least said binding agents directed against epitope 1 and epitope 2, which may be coupled via a linker, spacer. Upon binding CFTR, said multi-specific binding agent or multivalent ISVD may have an additive or synergistic impact on the stabilization or functionality of CFTR as compared to the monovalent or as compared to the combination of the single binding agents. In another embodiment, the invention provides a polypeptide comprising any of the immunoglobulin single variable domains according to the invention, either in a monovalent, multivalent or multi-specific form. Thus, polypeptides comprising monovalent, multivalent or multi-specific nanobodies are included here as non-limiting examples. One further aspect of the invention provides for a host cell comprising the binding agent, in particular the ISVD or active antibody fragment of the invention. The host cell may therefore comprise the nucleic acid molecule encoding said binding agent or ISVD or multi-specific or multivalent binding agent or ISVD. Host cells can be either prokaryotic or eukaryotic. The host cell may also be a recombinant host cell, which involves a cell which has been genetically modified to contain an isolated DNA molecule, nucleic acid molecule encoding the ISVD of the invention. Representative host cells that may be used to produce said ISVDs, but are not limited to, bacterial cells, yeast cells, plant cells and animal cells. Bacterial host cells suitable for production of the binding agents of the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. Animal host cells suitable for use with the invention include insect cells and mammalian cells (most particularly derived from Chinese hamster (e.g. CHO), and human cell lines, such as HeLa). Exemplary insect cell lines include, but are not limited to, Sf9 cells, baculovirus-insect cell systems (e.g. review Jarvis, Virology Volume 310, Issue 1, 25 May 2003, Pages 1-7). Alternatively, the host cells may also be transgenic animals.

Therapeutically Targeting CFTR

While nanobodies targeting extracellular epitopes of proteins involved in human diseases are currently being developed as potential drugs for a variety of human diseases^{[30]}, correcting CFTR folding defect requires intracellular action, and most likely at the level of the endoplasmic reticulum and/or the Golgi apparatus. Various tools for intracellular delivery have been developed over the years to introduce proteins (including antibodies) into cells in a functional state^{[31-33]}. When it is desired that the binding agent of the invention act intracellularly, the binding agent may require a cell penetrant carrier, which is capable of entering a cell through a sequence which mediates cell penetration (or cell translocation). So the binding agent further comprising a cell penetrant carrier involves the recombinant or synthetic attachment of a cell penetration sequence or molecule. Thus, the molecule (or polypeptide) may be further fused or chemically coupled to a sequence facilitating transduction of the fusion or chemical coupled proteins into prokaryotic or eukaryotic cells. Sequences facilitating protein transduction are known to the person skilled in the art and include, but are not limited to Protein Transduction Domains. It has been shown that a series of small protein domains, termed protein transduction domains (PTDs), cross biological membranes efficiently and independently of transporters or specific receptors, and promote the delivery of peptides and proteins into cells. Preferably, said sequence is selected from the group comprising TAT protein from human immunodeficiency virus (HIV-1), a polyarginine sequence, penetratin and a short amphipathic peptide carrier, Pep-1. Still other commonly used cell-permeable peptides (both natural and artificial peptides) are disclosed in Joliot A. and Prochiantz A. (2004) Nature Cell Biol. 6 (3) 189-193.

Still, the practical applications of intracellular delivery techniques into therapeutics will likely remain a significant challenge in the foreseeable future. Alternatively, gene therapy or the application of intrabodies (intracellular expression of nanobodies) may be considered. So another preferred embodiment relates to a vector for expression of the binding agent comprising an ISVD, or the multi-specific binding agent, preferably a viral vector, lentiviral, adenoviral or adeno-associated viral vector.

Where said (multispecific) CFTR binding agent is provided as a nucleic acid or a vector, it is particularly envisaged that the binding agent is administered through gene therapy. ‘Gene therapy’ as used herein refers to therapy performed by the administration to a subject of an expressed or expressible nucleic acid. For such applications, the nucleic acid molecule or vector as described herein allow for production of the CFTR binding agent, in particular the ISVD or intrabody, within a cell. A large number of methods for gene therapy are available in the art and include, for instance (adeno-associated) virus mediated gene silencing, or virus mediated gene therapy (e.g. US 20040023390; Mendell et al 2017, N Eng J Med 377:1713-1722). A plethora of delivery methods are well known to those of skill in the art and include but are not limited to viral delivery systems, microinjection of DNA plasmids, biolistics of naked nucleic acids, use of a liposome. In vivo delivery by administration to an individual patient occurs typically by systemic administration (e.g., intravenous, intraperitoneal infusion or brain injection; e.g. Mendell et al 2017, N Eng J Med 377:1713-1722). It is more particularly also envisaged that the binding agent is administered through delivery methods and vehicles that comprise nanoparticles or lipid-based delivery systems such as artificial exosomes, which may also be cell-specific, and suitable for delivery of the binding agents or multi-specific binding agents as intrabodies or in the form of DNA to encode said binding agent or modulator [48-49].

Such an alternative delivery method may comprise the use of lamellar lipid-build vesicle-like bodies (e.g. known as LAMELLASOMET1, for instance made as a synthetic mimetic based on phospholipids, with biophysical properties on the components of cystic fibrosis (CF) sputum, essentially identical to those of a natural lamellar body. The fact that such delivery vehicles seem clinically safe, and can be optimised to deliver active payloads such as gene therapies and anti-infectives provides for new avenues in biological delivery through the mucus and inside pulmonary cells of CF patients.

However, the use of small molecule compounds remains the method of choice for intracellular therapeutic targets, as membrane penetration can be an inherent property of the drug-like molecules. While classical experimental and computational approaches have failed to isolate small molecules with sufficient potential to stabilize CFTR^{[26]}, the crystal structures of complexes between NBD1 and various stabilizing nanobodies described here offer a new route for rational design of CFTR stabilizers (see below). As presented herein, the binding agents of the invention include small compounds, chemicals, nucleotides, peptides, peptide- or antibody-mimetics, as well as ISVDs or active antibody fragments, which specifically bind CFTR binding site or minimal epitope 1 and/or epitope 2, as described herein.

Another aspect of the invention relates to these binding agents of the invention, or the vectors expressing said binding agents, for use as a medicament, i.e. for therapeutic use. More specifically, said binding agents, multi-specific binding agents or vectors expressing said binding agents of the invention are for use in treatment of cystic fibrosis (CF) and/or CFTR-related disorders.

In fact, besides classic CF, non-classic CF and CFTR-related diseases all involve CFTR defects. The classic characteristics of CF are found in sinopulmonary disease, pancreatic insufficiency, male infertility, and elevated sweat chloride. The cornerstone of the diagnosis of CF is dysfunction of CFTR leading to disease in the sinopulmonary system, pancreas, sweat glands, and vas deferens. Over 300 mutations in the CFTR gene have been identified, leading to its dysfunction and a CF phenotype, which is determined by a gradient of CFTR dysfunction depending on the mutation type, as well as organ sensitivity to CFTR dysfunction. CFTR mutations have been divided into five different classes depending on the mechanism of mutation effect, from Class I (consisting of mutations that result in no meaningful CFTR protein production), to Class V (which results in decreased expression of normal CFTR protein). For an overview on the deficiencies per class, see for instance [51-52]. The vast majority of individuals with CF demonstrate a classic phenotype, with 85% or more being pancreatic insufficient and approximately 98% having elevated sweat chloride values. The disorder's most common signs and symptoms include progressive damage to the respiratory system and chronic digestive system problems. Most people with cystic fibrosis also have digestive problems. Some affected babies have meconium ileus, a blockage of the intestine that occurs shortly after birth. Other digestive problems result from a build-up of thick, sticky mucus in the pancreas. The pancreas is an organ that produces insulin (a hormone that helps control blood sugar levels). It also makes enzymes that help digest food. In people with cystic fibrosis, mucus often damages the pancreas, impairing its ability to produce insulin and digestive enzymes. Problems with digestion can lead to diarrhea, malnutrition, poor growth, and weight loss. In adolescence or adulthood, a shortage of insulin can cause a form of diabetes known as cystic fibrosis-related diabetes mellitus (CFRDM). Pancreatic phenotype can usually be predicted by CFTR genotype, with individuals carrying two “severe” mutations from classes I to III almost invariably being pancreatic exocrine insufficient. This is in contrast to CF lung disease, where a broad spectrum in severity is seen, but CFTR genotype is not predictive. Pulmonary phenotype appears to be most influenced by a combination of environmental factors and modifier gene.

‘Non-classical’ or ‘atypical’ CF disease is currently defined as the group that demonstrates a CF phenotype in at least one organ system and have normal (<40 mmol/L) or borderline (40-60 mmol/L) sweat chloride values. In general, individuals in this group tend to have pancreatic exocrine sufficiency and often have milder lung disease. Individuals with non-classic CF carry two CFTR mutations, at least one of which is usually a “mild” mutation resulting in partial CFTR expression and function. Examples of non-classical CF are Congenital bilateral absence of the vas deference (CBAVD) and Recurrent idiopathic pancreatitis. Even outside of the diagnosis of CF, other well-known disease entities can be influenced by CFTR genotype. While these diseases do not fit the criteria for CF or follow a Mendelian inheritance pattern, they are associated with CFTR mutations and therefore also defined as “CFTR-related diseases”, including Allergic Bronchopulmonary Aspergillosis (ABPA), chronic sinusitis, and idiopathic bronchiectasis. Although these illnesses appear to be influenced by CFTR dysfunction, they are most influenced by non-CFTR genes and environmental exposures.

The term “CFTR-related diseases”, “CFTR-related disorders”, or “CFTR-opathies” as used interchangeably herein hence include classic cystic fibrosis, and CFRDM, as well as non-classic CF, and other CFTR-related diseases such as ABPA, chronic sinusitis, and idiopathic bronchiectasis.

Drugs that target the underlying defect in the CFTR protein are called ‘CFTR modulators’. The three main types of modulators are potentiators, correctors, and amplifiers. Potentiators are drugs that help open the CFTR channel at the cell surface and increase chloride transport. Correctors are drugs that help the defective CFTR protein fold properly so that it can move to the cell surface. Amplifiers increase the amount of CFTR protein that the cell makes. Many CFTR mutations produce insufficient CFTR protein. If the cell made more CFTR protein, potentiators and correctors would be able to allow even more chloride to flow across the cell membrane. Furthermore, read-through compounds aim to allow full-length CFTR protein to be made, even when the RNA contains a mutation telling the ribosome to stop. RNA therapies aim to either fix the incorrect instructions in defective RNA, or provide normal RNA directly to the cell. Gene-editing techniques aim to repair the underlying genetic defect in the CF gene DNA. Gene replacement techniques aim to provide a correct copy of the CFTR gene.

There has been remarkable progress in the development of drugs to treat the underlying cellular processing and gating defects produced by mutations in CFTR. About 88% of the mutations include class II ‘F508del’, ‘N1303K’, and/or ‘I507del’ aka “processing mutations” wherein CFTR protein is created, but misfolds, keeping it from moving to the cell surface. Almost half of people with CF have two copies of the F508del mutation, which prevents the CFTR protein from forming the right shape. The protein with F508del (deletion of phenylalanine at position 508) is nearly completely degraded (99%) following polyubiquitination and recruitment of cytosolic proteasomes to the ER. Known that even wild-type CFTR protein shows very inefficient processing (as up to 75% of newly synthesized wild-type CFTR is degraded by the same pathway), this further illustrates that optimal protein folding is dependent not only on the primary amino acid sequence but also on other potentially manipulatable conditions. Since “misfolding” of the F508del CFTR and other class II mutants (e.g., G480C) does not completely abolish CFTR chloride conductance, therapies can be aimed primarily at overcoming the trafficking block, thereby permitting surface expression of the partially active mutant channel.

Correctors such as lumacaftor (VX-809; Vertex Pharmaceuticals) or tezacaftor (VX-661) help defective CFTR fold correctly, traffic to the cell surface, and stay there longer. Lumacaftor is capable of restoring ˜15% CFTR channel activity in primary respiratory epithelia expressing F508del-CFTR and is more selective for CFTR than most other folding correctors (for example, VRT-325 and corr-4a).

So, VX-809 mono- or combination therapy may restore function to a large number of rare CFTR mutations, aside its main action as a class II F508del-CFTR corrector. But, even with lumacaftor and tezacaftor, only about a third of the CFTR protein reaches the cell surface, so by itself it can't reduce the symptoms of CF. The F508del mutation showed not only misfolding of NBD1 (containing residue 508), but also instability of the NBD1-MSD2 interface, which may explain the rather modest rescue effect of most CFTR correctors, which target only a single defect. Thus, multidrug therapy combining a NBD1 domain stabilizer and a NBD1-MSD2 interface stabilizer is desired to overcome efficacy issues. Conceivably, the parallel targeting of multiple conformational defects by separate correctors will allow wild-type folding of the mutant protein and obviate the need for a potentiator. Remaining mutations lead to need for potentiators such as ivacaftor (VX-770; Kalydeco®), approved to treat cystic fibrosis caused by the G551D mutation and at least 38 other mutant CFTRs with defective channel gating, helps to open the CFTR channel and also help increase the function of normal CFTR.

The combination treatments using both, a corrector and a potentiator are for instance established by the combinations of lumacaftor/ivacaftor (Orkambi™) and tezacaftor/ivacaftor (Symdekon™), used to treat people with two copies of the F508del mutation. Tezacaftor/ivacaftor also can be used to treat people with a single copy of one of 26 specified mutations, regardless of the second mutation. However, the limited efficacy of lumacaftor/ivacaftor therapy in cell models and human clinical trials has motivated the development of corrector combination therapies in which a potentiator is combined with two correctors, each in principle targeting a distinct structural or dynamic defect in F508del-CFTR. The triple combination therapy called TrikaftaT® contains Elexacaftor (VX-445)+tezacaftor (VX-661)+ivacaftor (VX-770) as a combination of two CFTR correctors (Elexacaftor and tezacaftor), and one potentiator. Though, still full recovery of CFTR has not been observed so far, and still a population of F508del mutant CF patients is non-responsive. Alternatively, a concept combining potentiators (‘co-potentiator’) therapy for cystic fibrosis caused by difficult-to-treat CFTR mutations that appear to be refractory to treatment by single potentiators alone or in combination with correctors has been applied as well.

So, in the present application, as an alternative to purely administering one or another compound to a subject, one may also administer a composition comprising several types or several compounds. An embodiment of the invention provides for a composition, or a pharmaceutical composition, which contains the binding agents of the invention, including binding agents for the CFTR binding site of epitope 1 and/or epitope 2 of the invention. A further embodiment relates to said composition further comprising a small compound that is a CFTR corrector different from those binding agents of the invention. Said CFTR binding agents for epitope 1 and/or epitope 2 of the invention may act as a class II corrector, and may be present as a small compound, a chemical, a nucleotide, a peptide, a peptidomimetic, an antibody mimetic, an ISVD, or an antibody derivative such as an active antibody fragment. Further, said composition is characterized in that it will provide a synergistic effect on CFTR, and will be therapeutically useful. Said synergistic effect may be a synergistic effect on stabilization of CFTR, on folding of CFTR, as well as on ion channel activity of CFTR, or a combination of any of those effects which is resulting in an effect that is greater than the effects attained by the sum of the single compound administration. In a specific embodiment, said composition comprises a CFTR binding agent of the invention binding to epitope 1 and/or a CFTR binding agent of the invention binding to epitope 2, and a small compound CFTR corrector, and/or a CFTR potentiator. More specifically, wherein said CFTR corrector is lumacaftor, tezacaftor, elexacaftor, or another next-generation corrector, or a combination thereof, and said potentiator may be for instance but not limited to ivacaftor. In another specific embodiment, said composition comprises a CFTR binding agent of the invention binding to epitope 1 and/or a CFTR binding agent of the invention binding to epitope 2, and a small compound CFTR potentiator. More specifically, said CFTR potentiator may be ivacaftor, or a next-generation potentiator. Further embodiments involve a composition or pharmaceutical composition comprising a CFTR binding agent of the invention binding to epitope 1 and/or a CFTR binding agent of the invention binding to epitope 2, and additionally a different CFTR corrector and/or CFTR potentiator and/or a CFTR combination drug or mixture, wherein said combination drug or mixture may be selected from the list of lumacaftor/ivacaftor (Orkambi™), tezacaftor/ivacaftor (SymdekoN, or a potentiator combined with two correctors (e.g. Trikafta™), or a co-potentiator, or a combination of novel next-generation correctors and/or potentiators.

Such pharmaceutical compositions can be utilized to achieve the desired pharmacological effect by administration to a patient in need thereof. A “pharmaceutically or therapeutically effective amount” of compound or binding agent or composition is preferably that amount which produces a result or exerts an influence on the particular condition being treated. The CFTR binding agent or the pharmaceutical composition as described herein may also function as a “therapeutically active agent” which is used to refer to any molecule that has or may have a therapeutic effect (i.e. curative or stabilizing effect) in the context of treatment of a disease (as described herein). Preferably, a therapeutically active agent is a disease-modifying agent, and/or an agent with a curative effect on the disease. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52(5), 238-311), Strickley, R. G (“Parenteral Formulations of Small Molecule Therapeutics Marketed in the United States (1999)-Part-1” PDA Journal of Pharmaceutical Science & Technology 1999, 53(6), 324-349), and Nema, S. et al. (“Excipients and Their Use in Injectable Products” PDA Journal of Pharmaceutical Science & Technology 1997, 51 (4), 166-171). The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles. Such pharmaceutical composition comprising said CFTR binding agent may also concern a nanoparticle containing composition or lipid-based exosome delivery vehicle, as discussed herein. The CFTR binding agent or the pharmaceutical composition as described herein may act as a therapeutically active agent, when beneficial in treating CFTR-related diseases. The pharmaceutical composition as described herein may also comprise a multi-specific binding agent which may contain or be coupled to additional functional groups or moieties, advantageous when administrated to a subject. The compositions or pharmaceutical compositions as described herein may be applied for use as a medicament. More specifically, their use in the treatment of a patient with cystic fibrosis or a CFTR-related disorder is aimed for. Finally, due to the oral bio-availability of these new CF therapeutics, CFTR repair is no longer restricted to the lungs, as is the case for most DNA- and RNA-based mutation repair techniques, but is feasible in virtually all CF-relevant cell types, including bile ducts, intestine, sweat glands and immune cells.

Crystal Complexes

Another aspect of the invention relates to a complex comprising the CFTR, or at least the NBD1 domain, and a binding agent as described herein.

In a further embodiment, said complex is of a crystalline form. The crystalline allows to further use said the atomic details of the interactions in said complex as a molecular template to design molecules that will recapitulate the key features of the NBD1-binding agent interfaces. In the light of recent developments in computational docking and in pharmacophore building, the isolation of small compounds that can mimic protein-protein interface is becoming a realistic strategy. One of the challenges here is to develop small molecules that will not only bind given subdomains of NBD1, but also form the physical connection across these subdomains, which may require chemically linking molecules targeting different subdomains into a chimeric compound. In fact, the crystal structures of the complexes as presented herein allow direct modeling of the binding mode of each nanobody to FL-CFTR by superimposing the coordinates of NBD1 on the recently available cryo-EM structures of CFTR^{[2-4]}.

In a specific embodiment, the complex comprises CFTR or NBD1 protein and a CFTR binding agent which is an ISVD, or a multi-specific binding agent comprising an ISVD, in particular an ISVD comprising the CDRs as disclosed herein, or an ISVD comprising SEQ ID NO: 2-7 or a sequence with at least 90% amino acid identity thereof, or a humanized variant thereof. In a specific embodiment said CFTR/ISVD complex is crystalline.

So another embodiment relates to a composition in crystalline form comprising CFTR, or at least the NBD1 domain, and a binding agent, such as the nanobodies as presented herein, wherein the NBD1 domain is a domain with an amino acid sequence corresponding to 2PT-NBD1 (SEQ ID NO:58) and/or ΔRI-NBD1 (SEQ ID NO:59) or with a sequence with at least 90% identity thereof, and characterized in that the crystal is:
a crystal between the NBD1 domain and said binding agent in the space group C121, with the following crystal lattice constants: a=152.2 Å±5%, b=41.6 Å±5%, c=99.3 Å±5%, α=90°, β=120.56°, γ=90°, or
a crystal between the NBD1 domain and said binding agent in the space group C222₁, with the following crystal lattice constants: a=38.68 Å±5%, b=135.78 Å±5%, c=190.65 Å±5%, α=β=γ=90°, or
a crystal between the NBD1 domain, and said binding agent in the space group P2₁2₁2₁, with the following crystal lattice constants: a=64.49 Å±5%, b=118.15 Å±5%, c=180.21 Å±5%, α=β=γ=90°, or
a crystal between the NBD1 domain, and said binding agent in the space group P12₁1, with the following crystal lattice constants: a=80.94 Å±5%, b=55.19 Å±5%, c=114.99 Å±5%, α=90°, β=103.96°, γ=90°,

wherein the variation of crystal lattice constants may also be less than 5%, such as 4%, 3%, 2%, or 1%.

In another embodiment, said crystals as described herein has a three-dimensional structure wherein the crystal comprises an atomic structure characterized by the coordinates of PDB: 6GJS or a subset of atomic coordinates thereof. Alternatively, said crystal as described herein has a three-dimensional structure wherein the crystal comprises an atomic structure characterized by the coordinates of PDB: 6GJU or a subset of atomic coordinates thereof. Alternatively, said crystal as described herein has a three-dimensional structure wherein the crystal comprises an atomic structure characterized by the coordinates of PDB: 6GJQ or a subset of atomic coordinates thereof. Alternatively, said crystal as described herein has a three-dimensional structure wherein the crystal comprises an atomic structure characterized by the coordinates of PDB: 6GK4 or a subset of atomic coordinates thereof.

Another embodiment further discloses a CFTR NBD1 binding site, based on the information derived from said crystal structures, and hence consisting of a subset of atomic coordinates, present in the crystals as presented herein, wherein said binding site (epitope 1′) consists at least of the amino acid residues: 457, 459, 550-551, 576-581, 605-608, 610, 618, 625, 633 and 636, as depicted in SEQ ID NO:1 (human FL-CFTR), which in fact provides for the binding site of the T2a and D12 epitopes described herein, derived from the crystal of the complexes ΔRI-NBD1-D12-T4, 2PT-NBD1-T2a-T4, ΔRI-NBD1-D12-T8 and ΔRI-NBD1-D12-G3a. Alternatively, the binding site that is based on the information derived from said crystal structures, consists of a subset of atomic coordinates, present in the crystals as presented herein, wherein said binding site (epitope 1″) consists at least of the amino acid residues: 457-460, 550-551, 576-581, 605-608, 610, 618, 620, 625, and 633 as depicted in SEQ ID NO:1 (FL-CFTR), which in fact provides for the binding site of the T27 epitope described herein, derived from the crystal of the complex 2PT-NBD1-T27. Alternatively, the binding site that is based on the information derived from said crystal structures, consists of a subset of atomic coordinates, present in the crystals as presented herein, wherein said binding site (epitope 2′) consists at least of the amino acid residues: 469, 472, 474, 488-490, 494-499, 508-510, 553, 560, and 564 as depicted in SEQ ID NO:1 (FL-CFTR), which in fact provides for the binding site of the T4 epitope described herein, derived from the crystals of the complexes ΔRI-NBD1-D12-T4, and 2PT-NBD1-T2a-T4. Alternatively, the binding site that is based on the information derived from said crystal structures, consists of a subset of atomic coordinates, present in the crystals as presented herein, wherein said binding site (epitope 2″) consists at least of the amino acid residues: 472, 474, 490, 492, 494-499, 504, 506, 508-510, 560, and 564, as depicted in SEQ ID NO:1 (FL-CFTR), which in fact provides for the binding site of the T8 epitope described herein, derived from the crystal of the complex ΔRI-NBD1-D12-T8.

The binding sites epitope 1′ and epitope 1″ contain the minimal epitope residues of epitope 1, and all together, said minimal epitope 1 on NBD1 is bound by said Nbs capable of stabilizing wild type as well as F508del mutant CFTR proteins. Similarly, the binding sites epitope 2′ and epitope 2″ contain the minimal epitope residues of epitope 2, and all together, said minimal epitope 2 on NBD1 is bound by said Nbs capable of stabilizing at least wild type CFTR protein. Moreover, said stabilizing effect of said stabilizing NBs as used herein refers to an increase of more than 5° C. in melting temperature CFTR protein when bound, a newly technical effect that has never been observed for any CFTR binding agents.

Another aspect of the invention relates to a computer-assisted method of identifying, designing or screening for a modulator of CFTR, more specifically a molecule stabilizing the CFTR protein, wherein said modulator may be a stabilizer, a destabilizer, a channel activity antagonist, agonist, or inverse agonist, and is a CFTR binding agent selected from the group consisting of a small molecule compound, a chemical, a peptide, a peptidomimetic, an antibody mimetic, an ISVD, or an active antibody fragment, and comprising: i) introducing into a suitable computer program parameters defining the three-dimensional structure of the CFTR NBD1 binding site as disclosed herein; ii) creating a three-dimensional structure of a test compound in said computer program; iii) displaying a superimposing model of said test compound on the three-dimensional model of the binding site; and iv) assessing whether said test compound model fits spatially and chemically into the binding site.

In a specific embodiment, the computer-assisted method of identifying, designing or screening for a modulator of CFTR, the modulating activity is a stabilization of CFTR, more specifically the stabilizer is capable of increasing the thermal stability of CFTR with at least 5° C., resulting from an interaction with the NBD1 domain, and wherein said stabilizer is a CFTR binding agent selected from the group consisting of a small molecule compound, a chemical, a peptide, a peptidomimetic, an antibody mimetic, an ISVD, or an active antibody fragment, and comprising: i) introducing into a suitable computer program parameters defining the three-dimensional structure of the CFTR NBD1 binding site as disclosed herein; ii) creating a three-dimensional structure of a test compound in said computer program; iii) displaying a superimposing model of said test compound on the three-dimensional model of the binding site; iv) assessing whether said test compound model fits spatially and chemically into the binding site; and v) analysing the thermal stability as compared to the unbound or control CFTR complex via methods known in the art (see Examples for instance).

With a ‘control’ is meant herein a CFTR protein that is not bound to any compound, or that is bound to a molecule which has not thermostabilizing effect. A ‘control CFTR’ may be a wild-type or mutant CFTR, depending on the CFTR that is used for the method to identify the compound. A ‘control’ or ‘reference’ may also be a pool of data of control complexes or CFTR proteins. And a control should be treated or sampled or measured and analyzed in the same manner and conditions as the test sample or compound.

Rational Drug Design

Using a variety of known modelling techniques, the crystal structures of the present application can be used to produce models for evaluating the interaction of compounds with CFTR, in particular with the NBD1 domain. As used herein, the term “modelling” includes the quantitative and qualitative analysis of molecular structure and/or function based on atomic structural information and interaction models. The term “modelling” includes conventional numeric-based molecular dynamic and energy minimisation models, interactive computer graphic models, modified molecular mechanics models, distance geometry and other structure-based constraint models. Molecular modelling techniques can be applied to the atomic coordinates of the NBD1 domain, Nb complexes or parts thereof to derive a range of 3D models and to investigate the structure of binding sites, such as the binding sites with chemical entities. These techniques may also be used to screen for or design small and large chemical entities which are capable of binding the NBD1 domain and modulate the activity of CFTR. Such a screen may employ a solid 3D screening system or a computational screening system. Such modelling methods are to design or select chemical entities that possess stereochemical complementary to identified binding sites or pockets in the NBD1 domain. By “stereochemical complementarity” it is meant that the compound makes a sufficient number of energetically favourable contacts with the CFTR protein or with the NBD1 domain as to have a net reduction of free energy on binding to the CFTR protein or NBD1 domain. By “stereochemical similarity” it is meant that the compound makes about the same number of energetically favourable contacts with the NBD1 domain set out by the coordinates shown in PDB files: 6GJS, 6GJU, 6GJQ, and 6GK4. Stereochemical complementarity is characteristic of a molecule that matches intra-site surface residues lining the groove of the receptor site as enumerated by the coordinates set out in PDB files: 6GJS, 6GJU, 6GJQ, and 6GK4. By “match” we mean that the identified portions interact with the surface residues, for example, via hydrogen bonding or by non-covalent Van der Waals and Coulomb interactions (with surface or residue) which promote dissolvation of the molecule within the site, in such a way that retention of the molecule at the binding site is favoured energetically. It is preferred that the stereochemical complementarity is such that the compound has a K_{d} for the binding site of less than 10⁻⁴M, more preferably less than 10⁻⁵M and more preferably 10⁻⁶M. In a most particular embodiment, the K_{d} value is less than 10⁻⁸M and more particularly less than 10⁻⁹M. Chemical entities which are complementary to the shape and electrostatics or chemistry of the NBD1 domain or binding pockets of the NBD1 domain, characterised by amino acids positioned at atomic coordinates set out in PDB files: 6GJS, 6GJU, 6GJQ, and 6GK4 will be able to bind to the NBD1 domain, and when the binding is sufficiently strong, substantially modulate the activity of CFTR.

A number of methods may be used to identify chemical entities possessing stereochemical complementarity to the structure or substructures of the CFTR binding domain. For instance, the process may begin by visual inspection of a selected binding site in the NBD1 domain on the computer screen based on the coordinates in PDB files: 6GJS, 6GJU, 6GJQ, and 6GK4 generated from the machine-readable storage medium. Alternatively, selected fragments or chemical entities may then be positioned in a variety of orientations, or docked, within the selected binding site. Modelling software is well known and available in the art. This modelling step may be followed by energy minimization with standard available molecular mechanics force fields. Once suitable chemical entities or fragments have been selected, they can be assembled into a single compound. In one embodiment, assembly may proceed by visual inspection of the relationship of the fragments to each other on the three-dimensional image displayed on a computer screen in relation to the atomic coordinates of selected binding site or binding pocket in the CFTR binding site. This is followed by manual model building, typically using available software. Alternatively, fragments may be joined to additional atoms using standard chemical geometry. The above-described evaluation process for chemical entities may be performed in a similar fashion for chemical compounds.

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), Chemical Abstracts Service (Columbus, Ohio), the Available Chemical Directory (Symyx Technologies, Inc.), the Derwent World Drug Index (WDI), BioByteMasterFile, the National Cancer Institute database (NCI), Medchem Database (BioByte Corp.), ZINC docking database (University of California, Sterling and Irwin, J. Chem. Inf. Model, 2015), and the Maybridge catalogue. Once an entity or compound has been designed or selected by the above methods, the efficiency with which that entity or compound may bind to the CFTR (NBD1) domain or binding site can be tested and optimised by computational evaluation. For example, a compound that has been designed or selected to function as a NBD1 domain binding compound must also preferably traverse a volume not overlapping that occupied by the binding site when it is bound to the native NBD1 domain. An effective CFTR binding compound must preferably demonstrate a relatively small difference in energy between its bound and free states (i.e. a small deformation energy of binding). Thus, the most efficient CFTR binding compound should preferably be designed with a deformation energy of binding of not greater than about 10 kcal/mole, particularly, not greater than 7 kcal/mole. CFTR binding compounds may interact with, for instance but not limited to, the NBD1 domain in more than one conformation that are similar in overall binding energy. In those cases, the deformation energy of binding is taken to be the difference between the energy of the free compound and the average energy of the conformations observed when the compound binds to the protein. Further, a compound designed or selected as binding to the NBD1 domain may be further computationally optimised so that in its bound state it would preferably lack repulsive electrostatic interaction with the target protein.

Once a NBD1 domain or CFTR binding compound has been optimally selected or designed, as described above, substitutions may then be made in some of its atoms or side groups to improve or modify its binding properties. Generally, initial substitutions are conservative, i.e. the replacement group will have approximately the same size, shape, hydrophobicity and charge as the original group. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. It should, of course, be understood that components known in the art to alter conformation should be avoided. Such substituted chemical compounds may then be analysed for efficiency of fit to CFTR by the same computer methods described above.

Specific computer software is available in the art to evaluate compound deformation energy and electrostatic interaction. The screening/design methods may be implemented in hardware or software, or a combination of both. However, preferably, the methods are implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design. Each program is preferably implemented in a high level procedural or object-oriented programming language to communicate with a computer system. However, the programs can be implemented in assembly or machine language, if desired. In any case, the language may be compiled or interpreted language. Each such computer program is preferably stored on a storage medium or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

Compounds

The term “compound” or “test compound” or “candidate compound” or “drug candidate compound” as used herein describes any molecule, either naturally occurring or synthetic that may be tested in an assay, such as a screening assay or drug discovery assay, or specifically in the method for identifying a compound capable of modulating CFTR protein activity or stability. As such, these compounds comprise organic and inorganic compounds. The compounds may be small molecules, chemicals, peptides, antibodies or ISVDs or active antibody fragments. Compounds of the present invention include both those designed or identified using a screening method of the invention (as described herein for instance) and those which are capable of binding and modulating CFTR as defined above.

Compounds capable of binding and modulating CFTR may be produced using a screening method based on use of the atomic coordinates corresponding to the 3D structure of CFTR NBD1 complexes as presented herein, or based on a screening assay making use of the binding agents disclosed herein. The candidate compounds and/or compounds identified or designed using a method and or the binding agents of the present invention or derivatives thereof may be any suitable compound, synthetic or naturally occurring, preferably synthetic. In one embodiment, a synthetic compound selected or designed by the methods of the invention preferably has a molecular weight equal to or less than about 5000, 4000, 3000, 2000, 1000 or more preferably less than about 500 daltons. A compound of the present invention is preferably soluble under physiological conditions. The compounds may encompass numerous chemical classes, though typically they are organic molecules, preferably small organic compounds having a molecular weight of more than 50 and less than about 2,500 daltons, preferably less than 1,500, more preferably less than 1,000 and yet more preferably less than 500. Such compounds can comprise functional groups necessary for structural interaction with proteins, particularly hydrogen bonding, and typically include at least an amine, carbonyl, hydroxyl or carboxyl group, preferably at least two of the functional chemical groups. The compound may comprise cyclical carbon or heterocyclic structures and/or aromatic or polyaromatic structures substituted with one or more of the above functional groups. Compounds can also comprise biomolecules including peptides, saccharides, fatty acids, steroids, purines, pyrimidines, derivatives, structural analogues, or combinations thereof. Compounds may include, for example: (1) peptides such as soluble peptides, including Ig-tailed fusion peptides and members of random peptide libraries and combinatorial chemistry-derived molecular libraries made of D- and/or L-configuration amino acids; (2) phosphopeptides (e.g. members of random and partially degenerate, directed phosphopeptide libraries, (3) antibodies (e.g., polyclonal, monoclonal, humanized, anti-idiotypic, chimeric, and single chain antibodies, nanobodies as well as Fab, (Fab)₂, Fab expression library and epitope-binding fragments of antibodies); (4) non-immunoglobulin binding proteins such as but not restricted to avimers, DARPins and lipocalins; (5) nucleic acid-based aptamers; and (6) small organic and inorganic molecules.

Synthetic compound libraries are commercially available from, for example, Maybridge Chemical Co. (Tintagel, Cornwall, UK), AMRI (Budapest, Hungary) and ChemDiv (San Diego, Calif.), Specs (Delft, The Netherlands), ZINC15 (Univ. of California). In addition, numerous means are available for random and directed synthesis of a wide variety of organic compounds and biomolecules, including expression of randomized oligonucleotides. Alternatively, libraries of natural compounds in the form of bacterial, fungal, plant and animal extracts can be readily produced. In addition, natural or synthetic compound libraries and compounds can be readily modified through conventional chemical, physical and biochemical means and may be used to produce combinatorial libraries. In another approach, previously identified pharmacological agents can be subjected to directed or random chemical modifications, such as acylation, alkylation, esterification, amidification, and the analogues can be screened for CFTR modulating activity. In addition, numerous methods of producing combinatorial libraries are known in the art, including those involving biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide or peptide libraries, while the other four approaches are applicable to polypeptide, peptide, nonpeptide oligomer, or small molecule libraries of compounds. Compounds also include those that may be synthesized from leads generated by fragment-based drug design, wherein the binding of such chemical fragments is assessed by soaking or co-crystallizing such screen fragments into crystals provided by the invention and then subjecting these to an X-ray beam and obtaining diffraction data. Difference Fourier techniques are readily applied by those skilled in the art to determine the location within the CFTR structure at which these fragments bind, and such fragments can then be assembled by synthetic chemistry into larger compounds with increased affinity for CFTR. Further, compounds identified or designed using the methods of the invention can be a peptide or a mimetic thereof. The isolated peptides or mimetics of the invention may be conformationally constrained molecules or alternatively molecules which are not conformationally constrained such as, for example, non-constrained peptide sequences. The term “conformationally constrained molecules” means conformationally constrained peptides and conformationally constrained peptide analogues and derivatives. In addition, the amino acids may be replaced with a variety of uncoded or modified amino acids such as the corresponding D-amino acid or N-methyl amino acid. Other modifications include substitution of hydroxyl, thiol, amino and carboxyl functional groups with chemically similar groups. With regard to peptides and mimetics thereof, still other examples of other unnatural amino acids or chemical amino acid analogues/derivatives can be introduced as a substitution or addition. Also, a peptidomimetic may be used. A peptidomimetic is a molecule that mimics the biological activity of a peptide but is no longer peptidic in chemical nature. By strict definition, a peptidomimetic is a molecule that no longer contains any peptide bonds (that is, amide bonds between amino acids). However, the term peptide mimetic is sometimes used to describe molecules that are no longer completely peptidic in nature, such as pseudo-peptides, semi-peptides and peptoids. Whether completely or partially non-peptide, peptidomimetics for use in the methods of the invention, and/or of the invention, provide a spatial arrangement of reactive chemical moieties that closely resembles the three-dimensional arrangement of active groups in the peptide on which the peptidomimetic is based. As a result of this similar active-site geometry, the peptidomimetic has effects on biological systems which are similar to the biological activity of the peptide. There are sometimes advantages for using a mimetic of a given peptide rather than the peptide itself, because peptides commonly exhibit two undesirable properties: (1) poor bioavailability; and (2) short duration of action. Peptide mimetics offer an obvious route around these two major obstacles, since the molecules concerned are small enough to be both orally active and have a long duration of action. There are also considerable cost savings and improved patient compliance associated with peptide mimetics, since they can be administered orally compared with parenteral administration for peptides. Furthermore, peptide mimetics are generally cheaper to produce than peptides. Naturally, those skilled in the art will recognize that the design of a peptidomimetic may require slight structural alteration or adjustment of a chemical structure designed or identified using the methods of the invention. In general, chemical compounds identified or designed using the methods of the invention can be synthesized chemically and then tested for ability to modulate CFTR, using any of the methods described herein. The peptides or peptidomimetics of the present invention can be used in assays for screening for candidate compounds which bind to selected regions or selected conformations of CFTR. Binding can be either by covalent or non-covalent interactions, or both. Examples of non-covalent interactions include electrostatic interactions, van der Waals interactions, hydrophobic interactions and hydrophilic interactions.

When a compound of the invention interacts with CFTR, in particular interacts with the NBD1 domain of CFTR, it preferably “modulates” CFTR activity and/or stability. By “modulate” it is meant that the compound changes an activity and/or stability of CFTR by at least 10%, by at least 20%, by at least 30%, by at least 40%, or by at least 50%. Suitably, a compound modulates CFTR activity by increasing or decreasing the chloride channel activity of CFTR, preferably by increasing or decreasing its protein stability, more preferably its thermal stability. The ability of a candidate compound to increase or decrease the activity or stability of CFTR, can be assessed by any one of the CFTR assays known in the art, or as exemplified herein (see Example section). Compounds of the present invention preferably have an affinity for CFTR, preferably the NBD1 domain, sufficient to provide adequate binding for the intended purpose. Suitably, such compounds have an affinity (K_{d}) of from 10⁻⁵ to 10⁻¹⁵ M. For use as a therapeutic, the compound suitably has an affinity (K_{d}) of from 10⁻⁷ to 10⁻¹⁵ M, preferably from 10⁻⁸ to 10⁻¹² M and more preferably from 10⁻¹⁰ to 10⁻¹² M. As will be evident to the skilled person, the crystal structure presented herein has enabled, for the first time, new conformational states and dynamics of CFTR.

Screening Assays and Confirmation of Binding and Modulation

Screening assays for identifying compounds binding the CFTR binding site at epitope 1 or epitope 2, as described herein, may be obtained by a method making use of the ISVDs described herein binding to said epitopes, or making use of for instance low affinity mutants and derivatives thereof to further screen for new compounds that compete in CFTR or NBD1 binding, and with the functional property to increase activity and/or stability of CFTR in a similar manner as described herein.

One may envisage a screening assay in which the physical proximity of NBD1 (or CFTR) and a ‘minimal epitope 1- or minimal epitope 2-binding’ ISVD, in particular a Nb, is monitored. Measurement of the competitive binding capacity of the test compound is performed by measuring (physical) displacement of the epitope 1- or 2-binding ISVD relative to NBD1 (or CFTR) upon increasing concentration of the test compound or molecule. Non-limiting examples of ‘epitope 1- or epitope 2-binding’ ISVDs comprise the ISVDs as disclosed herein (SEQ ID NO:2-7), or variants thereof, for instance with reduced affinity as for instance depicted in SEQ ID NOs: 63-67, or further alternative variants as known by the skilled person thereof. The screening assay would require the following: both NBD1 (or CFTR) and the ‘epitope 1- or epitope 2-binding’ ISVD sequences may be engineered to bear a single accessible cysteine. The position of the cysteines are selected so as to be separated by a given distance (i.e. 50 Å) in NBD1-ISVD complex as seen in the crystal structure of the respective complex. Purified NBD1 will be labelled covalently on its accessible lone cysteine (i.e. position 519) with a commercially available thiol-reactive donor fluorophore. In parallel, the purified engineered ‘epitope 1- or epitope 2-binding’ ISVD will be labelled covalently on its lone engineered Cysteine (ie position 44) with a thiol-reactive acceptor fluorophore. The donor and acceptor fluorophore are selected to form a FRET (Forster Resonance Energy Transfer) pair, where light excitation of the donor leads to excitation and fluorescence emission of the donor when in close range (typically about 50 Å). Specifically, the pair is chosen to have a Ro (Foster radius) greater than the distance separating the two selected cysteines. In the absence of competition of the test compound, the NBD1 and ISVD will form a complex, leading to a strong FRET signal between the donor and acceptor fluorophores: the donor is excited at appropriate wavelength and the emission of the acceptor is measured. Upon addition of a competitor test compound, the ISVD and NBD1 will separate and the FRET signal will decrease. Binding of the competitor test compound molecule on NBD1 will therefore be measured as decrease in FRET signal, indicating the test compound as a suitable candidate CFTR binding agent as disclosed herein, i.e. with the functional properties for acting as a CFTR thermal stabilizer to increase a melting temperature with at least 5° C. as compared to a non-bound CFTR control.

The positive test compounds may be subjected to further confirmation of modulating or stabilizing CFTR, by co-crystallization of the compound with CFTR, or in particular with the NBD1 domain, and structural determination, as described herein. Additionally, the functional property can be tested by the thermal shift assay and DSF as described herein. Compounds designed or selected according to the methods disclosed herein are preferably assessed by a number of further in vitro and in vivo assays of CFTR interaction, in particular CFTR function to confirm their ability to affect CFTR protein maturation and its effect on functional ion channel transport activity.

For said screening assays, libraries may be screened in solution by the disclosed methods and/or methods generally known in the art for determining whether ligands competitively bind at a common binding site. Such methods may include screening libraries in solution, or on beads or chips. Where the screening assay is a binding assay, CFTR, in particular the NBD1 domain, may be joined to a label, as exemplified herein, where the label can directly or indirectly provide a detectable signal. Various labels include radioisotopes, fluorescent molecules, chemiluminescent molecules, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labelled with a molecule that provides for detection, in accordance with known procedures. A variety of other reagents may be included in the screening assay. These include reagents like salts, neutral proteins, e.g., albumin, detergents, etc., which are used to facilitate optimal protein-protein binding and/or reduce non-specific or background interactions. Reagents that improve the efficiency of the assay, such as protease inhibitors, nuclease inhibitors, antimicrobial agents, etc., may be used. The components are added in any order that produces the requisite binding. Incubations are performed at any temperature that facilitates optimal activity, typically between 4° C. and 40° C. Direct binding of compounds to CFTR, in particular its NBD1 domain, can also be done for example by Surface Plasmon Resonance (BIAcore).

Alternative CFTR Binding Agents for Modulating CFTR Activity

A final aspect of the invention provides for a binding agent specifically binding the CFTR binding site comprising amino acid residues 514, 515, 518, 522, 527, 530, 531, 534-537 as set forth in SEQ ID NO:1. Said binding site represents interaction with the NBD1 domain of CFTR without further stabilizing the protein, though said binding agent represents another potential modulator of CFTR. Indeed, specific binding to this site by, for instance, but not limited to Nb G3a, resulted in a thermal inactivation shift by 3.1° C. (FIG. 5b). So more specifically, said binding agents may be an ISVD comprising 4 framework regions (FR) and 3 complementarity determining regions (CDR) according to the following formula (1): FR1-CDR1-FR2-CDR2-FR3-CDR3-FR4 (1); wherein CDR1 consists of SEQ ID NO: 52; CDR2 consists of SEQ ID NO: 54; and CDR3 consists of SEQ ID NO: 56. Moreover, said ISVD may comprise SEQ ID: 50 (G3a Nb), or a sequence with at least 90% amino acid identity to SEQ ID NO: 50, or a humanized variant thereof. Another embodiment provides for a multi-specific binding agent, wherein at least one of the binding agents of the other binding sites of the invention is linked directly or via a spacer to the binding agent binding to the alternative binding site as presented herein.

In another embodiment, said binding agent may be used as a medicament, more specifically for treatment of cystic fibrosis. Alternatively said binding agent may be used as a tool for structural analysis, for diagnostic assaying, for detection of specific conformations of CFTR, among other applications. Another embodiment provides for the complex comprising said binding agent and the NBD1-domain of CFTR, which may be a crystalline complex. A specific embodiment provides for said crystal between the binding agent and the NBD1 domain, wherein said NBD1 domain is a domain with an amino acid sequence of SEQ ID NO: 58 or 59, or with a sequence with at least 90% identity thereof, and further characterized in that said crystal is a crystal in the space group P2₁2₁2₁, with the following crystal lattice constants: a=116.94 Å±5%, b=146.83 Å±5%, c=188.34 Å±5%, α=β=γ=90°.

The crystal as described herein may have a three-dimensional structure wherein the crystal i) comprises an atomic structure characterized by the coordinates of PDB: 6GKD or a subset of atomic coordinates thereof. Moreover, within said crystal, a binding site consisting of a subset of atomic coordinates, consists of the amino acid residues: 514, 515, 518, 522, 527, 530, 531, 534-537 as set forth in SEQ ID NO:1 as set forth in SEQ ID NO: 1, and wherein said amino acid residues represent the binding agent's CFTR binding site.

Another embodiment relates to a computer-assisted method of identifying, designing or screening for a modulator or binder of CFTR wherein said modulator is a binding agent selected from the group consisting of a small molecule compound, a chemical, a peptide, a peptidomimetic, an antibody mimetic, an ISVD, or an active antibody fragment, and comprises: introducing into suitable computer program parameters defining the three-dimensional structure of the binding site described herein; creating a three-dimensional structure of a test compound in said computer program; displaying a superimposing model of said test compound on the three-dimensional model of the binding site; and assessing whether said test compound model fits spatially and chemically into a binding site.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Generation of High Affinity Nanobodies Against NBD1

Two different llamas were immunized with 2PT-NBD1, a stabilized version of human CFTR NBD1 domain bearing the mutations S492P/A534P/1539T^{[19]}. Nanobodies were obtained after phage display selection, using established protocols^{[16]}. After two rounds of selection against 2PT-NBD1, a set of candidate binders was isolated. Among these nanobodies, we focused our biochemical characterization effort on 5 different nanobodies belonging to different sequence clusters, classified according to the sequences of the third complementarity determining region (CDR3) (FIG. 8).

Specific binding and apparent affinity of purified nanobodies D12, T2a, T27, T4, T8, and G3a to 2PT-NBD1 were confirmed by enzyme-linked immunosorbent assay (ELISA) using immobilized 2PT-NBD1. Dose response curves indicated a highly potent binding to 2PT-NBD1 and F508del-2PT-NBD1 (EC50 in the 1 nM to 50 nM range) for all nanobodies, except nanobody G3a, which displayed a weaker binding potency (FIG. 1a,e). Interestingly, F508del mutation drastically affected binding of nanobodies T4, T8 and to a lesser extent G3a, while nanobodies D12, T2a and T27 were not affected by the deletion (FIG. 1b-d).

Isothermal titration calorimetry (ITC) was used to characterize the thermodynamic parameters of the binding (FIG. 1e and FIG. 9). In each case the titrations were consistent with a 1:1 bimolecular association between nanobodies and monomeric 2PT-NBD1 with KD values of 54±10, 25±10, 37.9±7, and 39±2 nM with nanobodies T2a, T27, T4, and T8 respectively. Nanobody G3a bound 2PT-NBD1 with a lower affinity (1.1 μM±0.1 μM) which is consistent with our determination of apparent affinity by ELISA (FIG. 1a,e). These KD values are similar to those measured for other in vivo matured camelid heavy-chain antibodies interacting with their ligands^{[20]}. The thermodynamic parameters of the interaction of nanobodies with 2PT-NBD1 were determined based on these ITC measurements (FIG. 1e and FIG. 9).

### Example 2. Thermal Stabilization of NBD1 by Nanobodies

A range of mutations in NBD1 has already been described to improve thermal stability of this domain^{[5,9,21]} and more recently, He et al., showed that small molecules such as indole based-compounds, can counteract the folding defect of CFTR by stabilizing NBD1^{[14]}. In the context of F508del, specific NBD1 chaperones could be key molecules to overcome the deleterious effects of the mutation from a therapeutic perspective^{[22]}. We therefore evaluated the effect of our different nanobodies on apparent thermal unfolding (Tm) of 2PT-NBD1 by thermal shift assay (illustrated in FIG. 2a and FIG. 10). As shown in FIG. 2b, binding of nanobodies D12, T2a, T27, T4 and T8 led to strong stabilization of 2PT-NBD1, with increases of Tm of 13.8° C., 11.2° C., 11.9° C., 12.5° C. and 9.3° C. respectively. In contrast, G3a did not induce a significant shift in 2PT-NBD1 thermal stability. We confirmed the stabilizing effect using differential scanning calorimetry (DSC) for two nanobodies (T2a and T8) and obtained similar increases in Tm (FIG. 10). The stabilizing effects of D12 and T4 nanobodies were additive in combination, leading to an apparent melting temperature of the complex of 68° C., which is 24° C. higher than isolated 2PT-NBD1 (FIG. 2a). Interestingly, nanobodies D12, T2a and T27 stabilized F508del-2PT-NBD1 mutant to the same extent as 2PT-NBD1. In agreement with our binding data (FIGS. 1c,d), nanobodies T4 and T8 did not stabilize F508del-2PT-NBD1 (FIG. 2c).

### Example 3. Structures of Nanobody-NBD1 Complexes

In order to identify the molecular basis of NBD1 stabilization by nanobodies, we determined the crystal structure of each complex. Crystallization trials were performed using either 2PT-NBD1 or ΔRI-NBD1 constructs, as removal of the RI is known to improve the protein stability and favor crystallogenesis^{[7]}. We employed various crystallization strategies, using multiple stabilizing nanobodies at the same time and/or limited proteolysis to facilitate crystal formation. Structures were solved by molecular replacement using published structures of human NBD1 and nanobodies. Resolutions ranged from 1.9 to 3.0 Å (Table 1), allowing complete description of the binding interfaces.

As listed in Table 1, by multiplying nanobody combinations to help crystal formation, we solved the different interfaces several times under various crystallization conditions and in all cases showing a similar binding mode for a given nanobody. For example, nanobody D12 was observed in 3 different crystals: the ΔRI-D12-T4 complex (diffracting up to 1.95 Å), the ΔRI-D12-T8 complex (diffracting to 2.90 Å) and the ΔRI-D12-G3a complex treated with papain (diffracting to 3.00 Å). Comparison of the NBD1-D12 interfaces (residues with atoms closer than 4 Å) leads to C_{α}-atom root mean square deviations (RMSDs) below 0.41 Å.

In some cases, in situ limited proteolysis using papain or subtilisin A was required to generate diffracting crystals. Limited proteolysis is typically used to remove flexible loops that can prevent lattice formation^{[23]}. Analysis of the structures showed that the nanobodies themselves remained unaffected by protease treatment and that the complete binding interface is present and clearly seen in all structures. In contrast, significant portions of the NBD1 domain were cleaved, but the remaining fragment exhibited the typical NBD1 fold, albeit with some minor deviations far from the binding interface. For example, in the 2PT-NBD1-T2a-T4, papain cleavage at position K447 of 2PT-NBD1 led to the crystallization of a fragment of 2PT-NBD1 missing residues 389-447 and no electron density was observed for the likely flexible C terminal segment 638-646 and the loop 479-483 from the ABC_{β} subdomain. Nevertheless, the overall folding of 2PT-NBD1 polypeptide was highly similar to that of the previously published structure of NBD1 (PDB: 2PZE) with a root mean square deviation for the α-carbons of 0.78 Å. In addition, the regions involved in binding are highly similar in spite of protease treatment. For example we measured an overall RMSD below 1 Å for the C_{α}'s of residues involved in the binding interface shared (see below) by D12 (where no protease was used), T2a (treated with papain) and T27 (treated with subtilisin). Analysis of the different crystal structures revealed that 3 different epitopes are recognized by the 5 nanobodies characterized here.

### Example 4. A First Stabilizing Epitope Covers Several Subdomains

Nanobodies D12, T2a, and T27 recognize the same epitope (FIG. 3a) located on the edge of the α/β-core region, including the first residues of the Walker A motif and the last residues of the Walker B (FIG. 3b). Although these nanobodies belong to different sequence clusters (FIG. 8), their mode of binding is remarkably similar. While nanobodies typically recognize their cognate epitope via their highly variable and long CDR3^{[20,24],} these three nanobodies interact with NBD1 not only through residues from CDRs but also through their (conserved) framework regions. This observation explains the particularly large binding interfaces, extending over 1000 A2, with multiple contacts across the interface conserved among the nanobodies. In each of these three nanobodies, the CDR3 adopts a β-strand configuration, further extending the overall β-sandwich fold of the nanobody. The CDR3's contain one of two acidic residues that form an ionic interaction with K606 (FIG. 3c) in NBD1. Hydrogen bonds are formed between acidic side-chains and backbone amides, for example E608 in NBD1 with backbone from D109 in T27 or from D111 in D12 (illustrated in FIG. 3c), forming a tight set of polar interactions together with the aforementioned ionic bond. A set of hydrophobic interactions are observed towards the tip of the CDR3 loops of these nanobodies. This loop sits on top of the Walker A motif, where hydrophobic side chains from the nanobody occupy a small cavity present in the neighboring α/β-subdomain (see L108 in D12, FIG. 3c). We observe interaction between NBD1 and sidechains from the framework of the nanobody such as the conserved Y37 that forms a H-bond with the backbone amide from V580 in all of the structures of these three nanobodies. In addition, a hydrogen bond is observed between an Asp found at the tip of the CDR1 of nanobodies D12 and T2a and the backbone amides of G550 and G551 (slight differences are seen between the different solved structures). In summary, for these three nanobodies, the large interface can be similarly decomposed into four main contact sites, where specific interactions (electrostatic, hydrophobic and H-bond) are formed, extending over different subdomains of NBD1, covering over 30 Å in its longest axis.

TABLE 1
Data collection and refinement statistics

| | ΔRI-NBD1-D12-T4 | | 2PT-NBD1-T2a-T4 | | 2PT-NBD1-T27 | |
|---|---|---|---|---|---|---|
| PDB entry | 6GJS | | 6GJU | | 6GJQ | |
| Beamline | Proxima 2A | | Diamond I04 | | Diamond I02 | |
| Wavelength (Å) | 0.9789 | | 0.9795 | | 0.9795 | |
| Resolution range | 42.6-1.951 | (2.021-1.951) | 46.4-2.6 | (2.693-2.6) | 45.05-2.491 | (2.58-2.491) |
| (Å) | | | | | | |
| Space group | C 1 2 1 | | C 2 2 2₁ | | P 2₁ 2₁ 2₁ | |
| Unit cell | | | | | | |
| a, b, c (Å) | 152.2 41.6 99.3 | | 38.68 135.78 190.65 | | 64.49 118.15 180.21 | |
| α, β, γ (°) | 90 120.56 90 | | 90 90 90 | | 90 90 90 | |
| Total reflections | 144455 | (14336) | 103077 | (10201) | 445126 | (38800) |
| Unique reflections | 39029 | (3830) | 16001 | (1564) | 48669 | (4483) |
| Multiplicity | 3.7 | (3.7) | 6.4 | (6.5) | 9.1 | (8.7) |
| Completeness | 0.99 | (0.98) | 1.00 | (1.00) | 0.99 | (0.93) |
| Mean I/sigma(I) | 11.82 | (2.32) | 12.13 | (0.99) | 10.34 | (1.23) |
| Wilson B-factor | 27.57 | | 73.53 | | 50.54 | |
| (Å²) | | | | | | |
| R-merge | 0.07603 | (0.584) | 0.1204 | (1.649) | 0.1819 | (1.64) |
| R-meas | 0.08915 | (0.6811) | 0.1311 | (1.793) | 0.193 | (1.741) |
| CC1/2 | 0.997 | (0.746) | 0.997 | (0.565) | 0.995 | (0.467) |
| Reflections used | 39013 | (3830) | 15980 | (1559) | 48662 | (4482) |
| in refinement | | | | | | |
| Reflections used | 1952 | (192) | 799 | (78) | 2433 | (224) |
| for R-free | | | | | | |
| R-work | 0.1791 | (0.2576) | 0.2233 | (0.4685) | 0.1967 | (0.3100) |
| R-free | 0.2125 | (0.2783) | 0.2471 | (0.6226) | 0.2417 | (0.3794) |

| | | | |
|---|---|---|---|
| Number of non- | 3931 | 3254 | 10055 |
| hydrogen atoms | | | |
| Macromolecules | 3469 | 3154 | 9478 |
| Ligands | 33 | 13 | 124 |
| Protein residues | 458 | 421 | 1231 |
| RMS deviations | 0.014 | 0.012 | 0.015 |
| (bonds) (Å) | | | |
| RMS deviations | 1.68 | 1.66 | 1.97 |
| (angles) (°) | | | |
| Ramachandran | 98 | 96 | 97 |
| favored (%) | | | |
| Ramachandran | 2.2 | 3.7 | 3 |
| allowed (%) | | | |
| Ramachandran | 0 | 0 | 0 |
| outliers (%) | | | |
| Rotamer outliers (%) | 1.1 | 2.6 | 0.4 |
| Clashscore | 4.22 | 8.55 | 5.86 |
| Average B-factor | 35.25 | 77.33 | 51.12 |
| Macromolecules | 34 | 78 | 51 |
| Ligands | 39 | 81 | 84 |
| Solvent (%) | 44.50 | 67.01 | 50.01 |

| | |
|---|---|
| ΔRI-NBD1-D12-T8 | ΔRI-NBD1-D12-G3a |

| | | |
|---|---|---|
| PDB entry | 6GK4 | 6GKD |
| Beamline | Diamond I04 | Diamond I24 |

| Wavelength (Å) | 0.9795 | | 0.9686 | |
|---|---|---|---|---|
| Resolution range (Å) | 45.16-2.91 | (3.014-2.91) | 34.43-2.992 | (3.099-2.992) |

| | | |
|---|---|---|
| Space group | P 1 2₁ 1 | P 2₁ 2₁ 2₁ |
| Unit cell | | |
| a, b, c (Å) | 80.94 55.19 114.99 | 116.94 146.83 188.34 |
| α, β, γ (°) | 90 103.96 90 | 90 90 90 |

| Total reflections | 72866 | (656) | 293796 | (24776) |
|---|---|---|---|---|
| Unique reflections | 22020 | (190) | 65514 | (6045) |
| Multiplicity | 3.3 | (3.5) | 4.5 | (4.1) |
| Completeness | 0.99 | | 0.99 | (0.93) |
| Mean I/sigma(I) | 8.1 | (2.2) | 9.01 | (1.25) |
| Wilson B-factor | 49.80 | | 69.43 | |
| (Å²) | | | | |
| R-merge | 0.135 | (0.592) | 0.155 | (1.106) |
| R-meas | 0.161 | (0.703) | 0.1761 | (1.268) |
| CC1/2 | 0.985 | (0.620) | 0.992 | (0.4) |
| Reflections used | 21883 | (2167) | 65500 | (6045) |
| in refinement | | | | |
| Reflections used | 1086 | (102) | 3276 | (303) |
| for R-free | | | | |
| R-work | 0.2464 | (0.3037) | 0.2044 | (0.3206) |
| R-free | 0.2946 | (0.3657) | 0.2352 | (0.3525) |

| | | |
|---|---|---|
| Number of non- | 6870 | 20899 |
| hydrogen atoms | | |
| Macromolecules | 6563 | 20342 |
| Ligands | 70 | 323 |
| Protein residues | 894 | 2761 |
| RMS deviations | 0.015 | 0.013 |
| (bonds) (Å) | | |
| RMS deviations | 1.82 | 1.66 |
| (angles) (°) | | |
| Ramachandran | 95 | 98 |
| favored (%) | | |
| Ramachandran | 4.6 | 2.4 |
| allowed (%) | | |
| Ramachandran | 0 | 0 |
| outliers (%) | | |
| Rotamer outliers (%) | 3.6 | 2.1 |
| Clashscore | 11.18 | 7.20 |
| Average B-factor | 51.70 | 75.81 |
| Macromolecules | 52 | 76 |
| Ligands | 77 | 95 |
| Solvent (%) | 22.43 | 54.54 |

The location of these nanobodies completely overlaps (FIG. 11a) with that of the C-terminal regulatory extension (RE) observed in previously published structures of NBD1^{[25,26].} The RE segment, comprising residues 654-673 was removed from the 2PT-NBD1 construct used for immunization and characterization. While the functional role of the RE is still unclear, it has been described to be a very mobile domain^{[27]}. When we tested whether our nanobodies were able to bind a construct containing the RE (2PT-NBD1-RE), we still observed high-affinity binding for nanobodies D12, T2a and T27, albeit with decrease in apparent EC₅₀ compared to 2PT-NBD1 (FIG. 11b). This is consistent with RE being a dynamic region of CFTR.

### Example 5. A Second Stabilizing Epitope Includes F508

Although nanobodies T4 and T8 share no sequence similarity in CDR3, the crystal structures revealed that they bind NBD1 in the same location, a groove which includes the γ-phosphate switch loop/Q-loop (FIG. 3d,e) with an overall binding interface of over 900 A2. Here also we observed a non-classical nanobody-antigen binding mode in which the CDR3s contributed only a portion of the interface. Close inspection revealed that for both nanobodies 3 hydrogen bonds are formed between CDR3 residues and the Q-loop backbone, for example between Y103 and N105 in T4 and the carbonyl of 1497 (FIG. 3f). Y103 is also interacting with R553 in the α-subdomain of NBD1 through cation-π interactions. The other CDRs also participate in the interface, including a salt bridge observed between D54 in the CDR2 of T8 and K564 of NBD1, and a hydrogen bond observed between D55 of T4 and the backbone amide of F490. The conserved Y37 is also participating in the interface, in this case with the backbone carbonyl of P499. R57 in T4 (R58 in T8) makes a hydrogen bond with the backbone carbonyl of R560 and importantly also with the backbone carbonyl of F508. Indeed, one of the key features of the T4/T8 interface is that it directly involves F508. As shown in FIG. 3g, F508 is nestled inside a hydrophobic pocket formed by residues located between the second framework β-strand and CDR2, namely P47, L50, A60 and the Cβ of R58 in the case of T8, while for T4 the pocket is made up of L47, V50, A59, backbone atoms of V48 and Y48 as well as the Cβ of R57. In both cases the carbons of the aromatic ring of F508 are in ideal proximity to these side chains to form Van Der Waals interactions. Therefore, F508 is clearly part of the binding interface and it is thus not surprising that the binding of both T4 and T8 to F508del-2PT-NBD1 is drastically affected by F508 deletion (FIG. 1c,d).

### Example 6. Nanobody G3a Recognizes the Structurally Diverse Region

The non-stabilizing nanobody G3a (FIG. 2b) recognizes a third epitope located entirely in the so-called structurally diverse region (SDR) of NBD1 (FIG. 3h) with an overall surface of about 650 A2. On the nanobody, residues from the three CDRs (but not from the framework regions) contribute a series of hydrogen bonds (FIG. 3i). Residues S52, N54 and S56 in CDR2 form a tight cluster of hydrogen bonds with E514. CDR3 residues are involved in only two contacts (hydrogen bonds with K522 and E527), while CDR1 interacts more extensively, in particular as the formation of a short α-helix allows W31 to form cation-π interaction R518, which itself interacts with the backbone carbonyl of W31, and a salt bridge is observed between E535 from NBD1 and R27 from CDR1. This third epitope solely involves a single subdomain (spanning between residues 514 and 535), located on the tip of NBD1, unlike the other two epitopes in which the stabilizing nanobodies contact residues located far apart in NBD1, thus likely reducing conformational flexibility of NBD1.

### Example 7. Interaction of Nanobodies with Full-Length CFTR

As discussed above, thermal stabilization of NBD1 may provide a novel therapeutic route against the destabilizing F508del mutation. Considering that the stabilizing nanobodies described here were developed using isolated recombinant NBD1 domain for both immunization and selection, we investigated the ability of the nanobodies to recognize and stabilize the full-length CFTR (FL-CFTR). We thus tested the ability of these nanobodies to bind FL-CFTR in different assays. First, we used purified human CFTR to quantify binding potencies of representative nanobodies (one for each epitope) in an ELISA assay. When the nanobodies were immobilized and purified FL-CFTR was titrated, T2a, T8 and G3a were all able to bind with high affinity (FIG. 4a) reaching similar Bₘₐₓ values, demonstrating that each of the three epitopes identified was accessible in the context of the full-length protein. Interestingly, when performing the assay using immobilized CFTR and titrating the nanobodies (FIG. 4b), we observed that G3a and T8 reached Bₘₐₓ values lower than that observed for T2a (about 50% and 30% of T2a maximum signal respectively, FIG. 4c). This indicates that the epitopes of these two nanobodies are not accessible in a subset of population, suggestive of conformational diversity in the ensemble. We then used flow cytometry on permeabilized baby hamster kidney (BHK)-21 cells stably expressing human CFTR to establish whether the different nanobodies were capable of recognizing FL-CFTR in a cellular context. When comparing the fluorescence measured for the NBD1-specific nanobodies to that of the negative control (irrelevant nanobody, i.e. directed against a non-CFTR antigen) we observed strong increase in median signal, ranging from 3 fold to 5 fold over control (FIG. 4e,f), demonstrating all of these nanobodies also bind cellular CFTR. A similar behavior was observed for D12, T27 and T4 nanobodies (FIG. 12b,c). In order to test whether this signal was originating from binding to mature FL-CFTR we performed pull-down of cellular CFTR with T2a, T8 and G3a nanobody and analyzed the isolated CFTR by immunodetection after electrophoresis. Functional mature CFTR being fully glycosylated, electrophoresis allows to separate it from the intracellular immature CFTR. Mature CFTR with complex N-linked oligosaccharide chains migrates at an apparent molecular weight of 170 kDa (historically called band C) while immature core-glycosylated CFTR runs at a lower molecular weight (named band B). As shown in FIG. 4G, immunoblot analysis indicated that CFTR recognized by the three nanobodies shows an identical electrophoresis pattern as observed in whole cell lysate, where the large majority of the protein migrates to an apparent size of 170 kDa, which is expected for glycosylated CFTR (band C, highlighted in FIG. 4g), and thus mature protein. This was also observed for D12, T27 and T4 nanobodies (FIG. 12d).

In order to verify that the recognition of FL-CFTR by the nanobodies followed the binding modes observed on isolated NBD1, we performed flow cytometry experiment on 2PT-F508del expressing cells. This version of F508del is stabilized by three point mutations (I539T/S492P/A534P) which enable proper folding and maturation of CFTR, leading recovery of channel activity^{[19]}. As shown in FIG. 4H and FIG. 4I, nanobodies T2a and G3a bind efficiently this mutant, indicating that the native fold of NBD1 is present. However, nanobody T8 is not able to bind this mutant, most likely due to the lack of F508, which is involved in its epitope (FIG. 3g) and thus also required for binding of T8 to isolated NBD1 (FIG. 1d).

As our nanobodies are directed against NBD1 and that current models suggest that NBD1 and NBD2 must make contact in order to hydrolyze ATP^{[4,8]} we tested whether the different nanobodies could affect ATPase activity of CFTR. Incubation with saturating concentration of nanobodies D12, T2a, and T27 and strongly reduced ATPase activity to respectively 50%, 50% and 30% of PKA-phosphorylated CFTR (FIG. 5a), demonstrating nanobody interaction with the active, phosphorylated protein. ATPase activity was lowered to 60% in presence of nanobody T8, while G3a did not affect it.

ATPase activity was also used to measure thermal inactivation of CFTR, an assay shown to coincide with thermostability of NBD1^{[28]}. Addition of each of the different nanobodies shifted CFTR inactivation to higher temperature, up to 7° C. for the best stabilizing nanobody D12 (FIG. 5b), just as these nanobodies increased the apparent Tm of isolated NBD1 (FIG. 2b). We noted that, while ATPase activity of CFTR was not affected by the presence of G3a, thermal inactivation was shifted by 3.1° C. in the presence of G3a (FIG. 5b). This contrasts with the behavior observed by thermal shift assay where G3a did not affect the apparent Tm of isolated NBD1 (FIG. 2b,c). Nanobody stabilization of human FL-CFTR was confirmed with nanoscale differential scanning fluorimetry (nanoDSF). The analysis was performed with a stabilized version of human CFTR (stab-CFTR: 2PT/ARI/R1048A_1172X) allowing the production and purification of sufficient amount of functional human CFTR in detergent. As illustrated by the melting curves of stab-CFTR alone or in complex with T2a or T4 (FIG. 5c,d), we observed thermostabilization of 8° C. which is an example of a CFTR-specific reagent with strong stabilizing properties. Tm values obtained by nanoDSF are summarized in FIG. 5e.

### Example 8. Effect of Nanobodies on F508del Mutant CFTR Expression and Maturation

HEK293T cells expressing F508del-CFTR with an engineered extracellular 3HA tag were transiently transfected with the stabilizing Nbs, as well as control Nbs. The effect on F508del mutant CFTR expression and maturation was measured by flow cytometry, Western Blot and fluorescence microscopy. Next, the effect of the transfection of each Nb was measured in the absence or presence of the correctors VX-809 (lumacaftor) or VX-661 (Tezacaftor).

As shown in FIG. 14, the flow cytometry measurements illustrate that incubation of the cells with VX-809 corrector leads to a moderate increase in surface expression, as also observed upon transfection of the cells with stabilizing T2a, D12 or G5 Nbs. The G5 Nb (SEQ ID NO:5) has also been identified to interact with NBD1, at the same binding site as T2a, T27 and D12, and was taken along in structural and functional analyses (data not shown).

Furthermore, a combination of Nb and corrector presence showed a much stronger recovery of cell-surface expression, as shown in FIG. 14 (D), outlining a quantification of the normalized signals of FIG. 14A-C, with a synergistic effect of the combination of a F508del CFTR-stabilizing Nb and VX809 corrector treatments.

In addition, the western blot in FIG. 15 further demonstrated that band C, which represents the fully glycosylated mature CFTR present at the surface (and which is hence absent for untreated F508del-CFTR), is detectable after treatment of the cells with VX-809 or after transfection of the cells with T2a Nb. However, a much stronger band C is observed upon combination of the corrector VX-809 and T2a Nb, comparable to the level of WT CFTR present at the cell surface. Indeed, quantification of the band intensity (FIG. 15b) revealed a synergistic impact to establish a recovered level of mature CFTR protein, comparable to normal wild type levels, when VX-809 and T2a Nb were combined. On the other hand, no effect was observed when transfecting either the non-stabilizing G3a Nb or the T8 Nb which stabilizes WT CFTR, but not F508del CFTR.

Finally, immunostaining of cell-surface expression of WT and F508del CFTR-expressing HEK-293T cells was performed to visualize that the protein was present with a corresponding level at the surface: moderate staining was observed in F508del-CFTR cells treated with VX-809 or transfected with T2a Nb, but a wild-type-like staining level was observed when treated with both.

As a second example, the combination of the same T2a Nb with the corrector VX-661 was analysed. FIG. 17 shows the CFTR protein bands on Western blot as well as the quantification of the signals for the mature protein band. From this first analysis, only a minor effect could be observed for the addition of VX-661 or addition of T2a Nb as compared to their respective controls, but still a synergistic effect was observed when both the corrector VX-661 and T2a Nb were used in combination. The recovery of the level of mature CFTR was lower as compared to wild type levels, though the effect is clear. Further repetition and tests may be necessary to confirm these differences.

So, there is a clear increase in protein maturation when the F508del CFTR-expressing cells are treated with known corrector drugs or with the novel Nbs as presented in this application. Moreover, a synergistic effect on protein maturation is observed when a combination of both, drugs and Nbs, is simultaneously applied.

### Example 9. In Cellulo CFTR Function Analysis in the Presence of Nbs and/or Small Molecules

To study CFTR function in cells, we used a HS-YFP quenching assay where changes in fluorescence of halide sensitive YFP (HS-YFP) reflect halide entrance through CFTR. HEK293T cells stably expressing F508del-CFTR (or WT CFTR as a control) and a modified YFP were transiently transfected with pcDNA3-based plasmid coding either for a stabilizing nanobody (T2a, D12 or T27) or a control nanobody (not binding F508del-CFTR) and incubated with 3 μM VX809 corrector or 0.06% DMSO for 24 h. Before addition of iodide, cells are stimulated by 10 μM forskolin and 3 μM VX770 potentiator for 20 mins and YFP fluorescence signal is measured (excitation 485, emission 535 nm) over a period of 4 seconds.

The results are shown in FIG. 18, and demonstrate that the mutant F508del CFTR expressing cells with a control (non-binding Nb) almost do not show any quenching of the signal, whereas the F508delCFTR expressing cells treated with stabilizing Nb T2a, D12, and T27 as well as the treatment with control NB+VX809 corrector lead to a quenching of YFP, indicating partial functionality of the CFTR activity. Moreover, a combination treatment of said stabilizing Nbs and the VX809 corrector has a further synergistic impact on the functionality of the CFTR channel in said cells, restoring the function to wild type CFTR levels.

### Example 10. Forskolin Induced Organoid Swelling Assay

Intestinal organoids are 3D epithelial structures grown from a single Lgr5+ stem cell (originating from the crypts of the GI-tract) with an internal lumen that recapitulates key features of the intestinal tissue architecture. When differentiated, the organoids form villus and crypt-like structures. CFTR is located at the apical membrane lining the internal lumen and its activation leads to rapid organoid swelling, in direct correlation with the amount of functional CFTR. This model system thus provides a physiologically relevant assay to evaluate the potential of new therapies with high translational value. CF patient-derived organoids expressing F508del-CFTR from two alleles were transduced with lentiviral vectors encoding the sequence of either a stabilizing nanobody (T2a) or a control nanobody. 24 h before FIS assay, corrector 3 μM VX-809 or DMSO was added. Organoids were stimulated by Fsk (0,8 μM) and 3 μM VX-770 just before FIS. CFTR response was followed by measuring the relative increase in surface area of the organoids over a period of 2 h. The results are presented in FIG. 19 and reveal that the organoids transduced with stabilizing Nb led to a significantly increased CFTR functionality over time, whereas the increase measured for the organoids transduced with non-stabilizing control Nb showed a stagnation of the CFTR response upon treatment with small molecules corrector VX809 after approx. 1 h. In conclusion, this ‘gold standard’ test for clinical application of CFTR agents reveals therapeutic potential for an improved combination therapy applying small molecules correctors, such as VX809 and/or VX-661, with the stabilizing CFTR binding agents of the present invention.

Conclusions of the Examples.

This study demonstrates that large thermal stabilization (>10° C. increase in Tm) of isolated NBD1 and of full-length CFTR bound to a stabilizing Nb versus their non-bound form, under the same testing conditions, can be achieved with antibodies. The stabilizing nanobodies bind distinct, conformational and non-overlapping epitopes, with common features. For instance, the interaction interfaces span several subdomains of NBD1, covering relatively large distances (over 30 Å). As such, both families of stabilizing nanobodies (targeting epitope 1 or 2), provide, upon binding, a physical connection between the α-subdomain and the α/β-subdomain of NBD1 (FIG. 3b,e). This exogenous bridging of NBD1 tertiary structure is likely to be responsible for the large stabilizing effect observed. In contrast, the non-stabilizing nanobody G3a does not mediate long range connection, instead binding solely to a unique subdomain (SDR). Importantly, binding to two different stabilizing epitopes act on the protein in distinct ways, as incubating NBD1 with D12 and T4 produced additive effects (FIG. 2a), suggesting that several sites could be targeted to maximize therapeutic benefit.

Superimpositions of our crystal structures of the complexes show that the epitope recognized by G3a should be accessible in CFTR (FIG. 6a and FIG. 13a) with no visible steric hindrance, correlating with the efficient recognition observed in flow cytometry, ELISA and pull-down experiments with this nanobody.

Nanobodies D12, T2a, and T27 are predicted to bind CFTR between NBD1 and NBD2 (FIG. 6b). The structure of dephosphorylated human CFTR (PDB: 5UAK) displays sufficient spread between the two NBDs to allow positioning of the nanobody (a slight increase in the opening could be required to alleviate any minor steric overlap). In contrast, the closing of the NBDs observed in the structure of phosphorylated zebrafish CFTR (PDB: 5W81) is expected to prevent binding of such nanobody (FIG. 13b). This agrees well with the strong decrease of ATPase activity observed in the presence of these nanobodies which, upon binding would thus prevent the NBD1-NBD2 interaction required for enzymatic activity. Therefore, while theses nanobodies may be able to stabilize NBD1, they could also hinder channel function. The use of a small molecule mimetic might circumvent such steric limitation, and rational drug design may require carefully taking into account the structures of the different states of CFTR, which are currently emerging.

Superimposing the crystal structures of NBD1-T4 or NBD1-T8 onto the cryo-EM structures of FL-CFTR suggest that these nanobodies should not recognize the full-length protein (FIG. 6c and FIG. 13c). Indeed, T4 and T8 completely overlap with the position of the coupling helix of the ICL4, and also with ICL1 and surrounding helices. For ABC proteins in general, ICL4 is considered to be the main interaction site between NBD1 and TMD2, yielding a stable TMD-NBD1 interface.

Moreover, while F508 is completely solvent exposed in the isolated NBD1 domain, it becomes completely buried in the NBD1-ICL interface observed in the cryo-EM structures (and thus not available for the nanobodies), while our data have demonstrated that interaction with F508 is strictly required for binding by T4 or T8. Based on these structural data, one would predict that epitope of T4/T8 should not be accessible in FL-CFTR, although our experiments clearly demonstrate that these nanobodies bind mature CFTR, either isolated or in cellular membranes.

Altogether our data would imply that NBD1 must detach from ICL4 and reorient in a manner that allows binding of a ^{˜}15 kDa nanobody (schematized in FIG. 7). Interestingly, structural analysis of the interface reveals that the NBD1-TMD interface is significantly weaker than the NBD2-TMD interface, mainly because NBD1 is devoid of usually conserved NBD structural features, namely the S5 β-strand and the h2 α-helix, leading to a reduced interaction surface^{[35]}. While this has been previously described as a structural weakness that will render the channel sensitive to modification of the interface (i.e. F508del), it could also be that the reduced interface was evolved to allow undocking of NBD1 for a functional reason. While undocking of NBD1 from ICL4 may appear surprising, it is supported by previous work. Earlier studies have shown that cysteines introduced in NBD1 (at position 508) and ICL4 (at position 1068) which are separated in the cryo-EM structure by about 7 Å (C_{β}-C_{β} distance) can be efficiently bridged using crosslinkers of lengths ranging from 4 Å to 24 Å, which could agree with domain motion^{[34]}. Furthermore, crosslinking these two positions with the short reagent 1,1-methanediyl bismethanethiosulfonate (M1M) leads to inhibition of channel gating, which can be reverted by reducing agent, suggesting that a conformational rearrangement of the NBD1-TMD interface may be required for proper function. In addition, HDX experiments on the bacterial ABC homodimeric transporter BmrA have shown that the ICD2 peptide (corresponding to ICL4 in CFTR) exchanges extensively with the solvent, indicating that is not permanently buried as observed in crystal structure of homologs^{[35]} which suggests that NBD undocking may be happening in other members of the ABC family.

In conclusion, nanobody binding at the NBD1-TMD interface implies that this highly important region is more dynamic than previously appreciated, and therefore suggests the necessity to reconsider how mutations affect the integrity of NBD1 and that of the interface in a physiopathological context. We surmise that a new perspective on the dynamics of the interface should have important consequences for therapeutic strategies aimed at modulating its stability.

Methods

Human NBD1 Expression and Purification

Human ΔRI-NBD1 (residues 387-646, A405-436; SEQ ID NO:59) construct was obtained from Arizona State University Plasmid Repository (clone id: 287374), 2PT-NBD1 mutants (residues 387-646 containing the mutations S492P, A534P, I539T; SEQ ID NO:58), 2PT-NBD1-RE (2PT-NBD1 with residues 387-678) were constructed using WT-NBD1 construct from ASU (clone id: 287401). Mutations were introduced by PCR using PfuUltra high-fidelity DNA polymerase from Agilent (catalogue number: 600382) and sequences were confirmed by automated DNA sequencing (UNC-CH Genome Analysis Facility). Proteins were expressed as N-terminal, His6-SUMO fusion proteins in Escherichia coli (BL21(DE3) pLysS cells, Millipore) as described in [5,11] with the following modifications. Cells were lysed using a French press and recombinant proteins were purified by nickel ion affinity chromatography (HisTrap HP, 1 ml—GE Healthcare). The His6-SUMO tag was removed using Ulp1 protease at 1/100 weight/weight ratio during 20 min on ice. Then, the cleaved fraction was separated by affinity chromatography (HisTrap HP, GE Healthcare) and further purified by gel filtration on a Superdex 200 10/300 column (GE Healthcare) equilibrated with storage buffer (20 mM Hepes pH 7.5, 150 mM NaCl, 10% (w/v) glycerol, 10% (w/v) ethylene glycol, 2 mM ATP, 3 mM MgCl2, 1 mM Tris(2-carboxyethyl)phosphine (TCEP)). Protein concentration was determined using Coomassie Plus (Bradford) Assay Kit (Thermo Scientific).

Nanobody Cloning and Expression and Purification

Nanobodies were cloned in pXAP100 vector. pXAP100 is similar to pMES4 (genbank GQ907248) but contains a C-terminal His6-cMyc tag and allows cloning of the VHH repertoire via Sfil-BstEll restriction sites. Twin-Strep nanobodies were design as follow: the synthetic gene encoding full-length T8 nanobody fused to a C-terminal cleavage site for human rhinovirus 3C (P3C, LEVLFQGP (SEQ ID NO:60)), a cMyc tag (EQKLISEEDL (SEQ ID NO:61)) and a Twin-Strep-tag (WSHPQFEKGGGSGGGSGGSAWSHPQFEK (SEQ ID NO:62)) instead of the His6-cMyc tag was synthesized by Eurofins Genomics and then recloned into pXAP100 vector using NotI/EcoRV restrictions sites. Then, the modified vector was digested with Sfi/NotI to allow insertion of nanobodies T2a, T4, T27, G3a, or D12 in frame with the P3C-cMyc-TwinStrep sequence. All constructs were verified by sequencing (Eurofins Genomics). Nanobody expression and purification were performed as previously described^{[16]}. Briefly, nanobodies were produced in Escherichia coli (BL21(DE3) pLysS cells, Millipore), purified from the periplasmic extract via either HisPur Ni-NTA resin (ThermoScientific) or Strep-Tactin XT Superflow resin (iba LifeScience) followed by a size exclusion chromatography on a Superdex 200 Increase 10/300 GL (GE Healthcare) equilibrated in 20 mM HEPES pH 7.5, 150 mM NaCl, and 10% (w/v) glycerol.

NBD1 ELISA Assay

For dose-response assays, Nunc MaxiSorp 96-well plates (ThermoScientific), were coated with 5 μg/ml NeutrAvidin Biotin-Binding protein (ThermoScientific) overnight at 4° C. and blocked 2 h at room temperature (RT) with 2% milk in phosphate-buffered saline (PBS). Each new reagent addition was preceded by three washes with 200 μl of NBD1 buffer (20 mM HEPES pH 7.5, 150 mM NaCl, and 10% (w/v) glycerol, 10% (w/v) ethylene glycol, 2 mM ATP, 3 mM MgCl₂). Then, biotinylated purified NBD1 proteins at 5 μg/ml were immobilized 30 min at RT followed by 1 h RT incubation with 100 μl various concentrations (0-20 μg/ml) of purified nanobodies. Signal detection was followed using His-tag specific antibody (Invitrogen, catalogue number: MA1-135, 1:3000 dilution) to detect the nanobodies and secondary antibody anti-mouse coupled to horse radish peroxidase (HRP) (Millipore, catalogue number: AP308P, 1:5000 dilution). 50 μl of 1-Step UltraTMB-ELISA (ThermoScientific) was used as a substrate for the peroxidase and intensity of the reaction was proportional to absorbance measured at 450 nm with SynergyMx (BioTek) after addition of 50 μl H₂SO₄ at 1M.

Thermal Shift Assay (DSF)

Solutions of either 2PT-NBD1 or F508del-2PT-NBD1 (10 μM final concentration), nanobodies (30 μM final concentration) and 2.5× or 5× concentrated SYPRO Orange Protein Stain (Molecular Probes) diluted in 20 mM HEPES pH 7.5, 150 mM NaCl, 3 mM MgCl₂, 2 mM ATP and 10% (w/v) glycerol, 10% (w/v) ethylene glycol, were added to the wells of a 96-well PCR plates type BR white (VWR) in a final volume of 25 μl. Plates were sealed with EasySeal sheets (Molecular dimensions) and spun 2 min at 900× g. SYPRO orange fluorescence was monitored in CFX96 Touch Real-Time PCR Detection System (Bio-Rad) using FRET scan mode from 10 to 80° C. in increments of either 1° C. or 0.2° C.

Isothermal Titration Microcalorimetry (ITC)

Interactions between nanobodies and 2PT-NBD1 was carried out on NanoITC system (TA Instruments) in 0.165 ml cells at 20° C., 300 rpm syringe stirring. Proteins were extensively dialyzed in 20 mM Hepes buffer pH 7.5, 150 mM NaCl, 10% (w/v) glycerol, 10% (w/v) ethylene glycol, 2 mM ATP and 3 mM MgCl₂ for 16 h at 4° C. Heat of dilution from control experiments of each nanobody titrated into buffer was subtracted from the titration into 2PT-NBD1. Data were integrated analyzed with Origin 7.0 software (Origin Lab Corp.).

Differential Scanning Calorimetry (DSC)

Calorimetry was performed on the MicroCal VP-Capillary DSC system (Malvern Instruments Ltd). Data were analyzed using the MicroCal Origin software and buffer-buffer heat capacity curve was subtracted from each protein curve. Purified 2PT-NBD1 was incubated with 1.2 molar excess of each nanobody in 20 mM Hepes pH 7.5, 150 mM NaCl, 10% (w/v) glycerol, 10% (w/v) ethylene glycol, 2 mM ATP, 3 mM MgCl₂, and incubated for 1 h on ice.

Crystallization Trials and In Situ Proteolysis

For each complex formation, nanobodies were SEC purified the day before in 20 mM Hepes pH 7.5, 150 mM NaCl, 10% (w/v) glycerol and mixed with freshly SEC purified NBD1 with 1.2 molar excess of nanobodies, and keeping 2 mM ATP, 3 mM MgCl₂ and 1 mM TCEP final concentrations. Protein complexes were incubated 1 h on ice and then concentrated onto 30 kDa MWCO Amicon concentrator (Millipore) until protein concentration reaches 10-18 mg/ml. Proteases from Floppy Choppy kit (Jena Biosciences), either papain or subtilisin A, at a concentration of 1 mg/ml were added to the purified protein on ice immediately prior to crystallization trials at a ratio of 1 μg protease per 200 μg of protein complex. Crystallization was performed in sitting drops at RT, adding 100 nl of the protease/protein mixture to 100 nl of the precipitant and were set up immediately using Mosquito robot (Art Robbins). For each NBD1-nanobody complex an initial screen of seven commercial screening kits was used (HR-Index, HR-Crystal Screen I&II, MD-Proplex, MD-PACT premier, MD JCSG+, MD-Clear Strategy I, MD-Structure Screen I&II). Crystallization plates were incubated at 20° C. Single crystals were mounted in CryoLoops (Molecular Dimensions Ltd) and flash-frozen in liquid nitrogen.

Crystal Structure Determination

Native high-resolution X-ray diffraction data were recorded on synchrotron beamline PX2 at SOLEIL in St Aubin, France, with an EIGER X 9M detector for the ΔRI-NBD1-D12-T4 complex, on beamline i04 at the Diamond Light Source in Didcot, United Kingdom, with a PILATUS 6M detector for the 2PT-NBD1-T2a-T4 and ΔRI-NBD1-D12-T8 complexes, on beamline i02 at the Diamond Light Source in Didcot, United Kingdom, with a PILATUS 6M detector for the 2PT-NBD1-T27 complex, and on beamline i24 at the Diamond Light Source in Didcot, United Kingdom, with a PILATUS 6M detector for the 2PT-NBD1-T27 complex. Data were integrated and scaled using the XDS program^{[37]}. For each NBD1-nanobody complex, the dataset was solved by molecular replacement using Molrep^{[38]}. Subsequently, several cycles of model building, using COOT 39, combined with refinement using BUSTER 2.10.1 40 were conducted. Finally, structure validation was performed with MolProbity^{[41]}. Figures and structural comparisons of the different NBD1-nanobody complexes with the human NBD1 structures previously published (PDB: 2PZE and 2PZF 7, PDB: 2BBO 25, PDB: 1XMJ and 1XMJ 6) were prepared using UCSF Chimera^{[42]}. The atomic coordinates and structure factors reported in this paper were deposited in the Protein Data Bank (PDB) with accession numbers PDB: 6GJS, 6GJQ, 6GJU, 6GK4, and 6GKD.

Human CFTR Expression and Purification

Two sources of protein were used. A stabilized version of human CFTR protein (stab-CFTR:2PT/ΔRI/R1048A_1172X) was stably expressed into BHK-21 cells (ATCC; CCL-10) with pNUT vector (Palmiter, 1987) which were maintained in methotrexate containing medium43.44 wt-CFTR fused to enhanced green fluorescent protein (His10-SUMO*-CFTRFLAG-EGFP) was stably expressed in human embryonic kidney (HEK) 293 cell line D165 45 and was PKA phosphorylated with protein kinase A catalytic subunit and affinity purified to homogeneity using NiNTA resin (Qiagen) according to manufacturer-recommended procedures in 50 mM HEPES pH 7.5, 0.15 M NaCl, 10% glycerol, 2.5 mM MgCl₂, 2 mM ATP, 0.35 M imidazole, 0.01% Decyl Maltose Neopentyl Glycol (DMNG—Anatrace), 1 mM dithiothreitol. Cells were cultured according to standard mammalian tissue culture protocols including testing for mycoplasma.

Full-Length CFTR ELISA

Strep-Tactin XT coated microplate (iba Life Science) was coated overnight at 4° C. with Twin-Strep-tagged nanobodies (5 μg/ml). Plate was blocked with 4% milk for 2 h at RT. Then different concentrations of CFTR (10⁻¹⁰ to 10⁻⁸ M) were incubated for 2 h at 4° C. CFTR binding was detected with monoclonal antibodies L12B4, MM13, 154, 660, 570, 596 specific to CFTR obtained from the CFTR Antibody Distribution Program (http://cftrantibodies.web.unc.edu/available-antibodies)46 and then anti-mouse-HRP antibody (Millipore, catalogue number: AP308P, 0.5 μg/ml) for 1 h 30 min at 4° C. For Bₘₐₓ determination Pierce Nickel Coated Plate (ThermoScientific) was coated 1 h at 4° C. with CFTR (8 μg/ml). Plate was blocked with 4% milk for 2 h at 4° C. Then different concentrations of nanobodies (10⁻⁹ to 10⁻⁶ M) were incubated for 2 h at 4° C. Nanobody binding was detected with Myc-tag specific antibody (Sigma, catalogue number: C3956, 0.5 μg/ml) and then anti-rabbit-HRP antibody (Cell Signaling, catalogue number: 7074S, 1:1000 dilution) for 1 h 30 min at 4° C. Between each step wells were washed 3 times by aspiration with 50 mM HEPES pH 7.5, 150 mM NaCl, 10% glycerol, 2 mM ATP, 2.5 mM MgCl₂, 0.01% DMNG (Anatrace). Incubations were performed in the same buffer with 0.4% milk. Reaction was visualized by using 1-Step Ultra TMB-ELISA (ThermoScientific) and stopped with H₂SO₄ (500 mM final). Absorbance was measured at 450 nm using SynergyMx (BioTek).

Flow Cytometry

Parental BHK-21 cells (ATCC; CCL-10) and cells stably overexpressing human wt-CFTR^{[43,44,47]} or 2PT-F508del-CFTR, as described above, were permeabilized with 0.01% n-Dodecyl-β-D-Maltopyranoside β-DDM—Inalco) at least for 2 h on ice. In the meantime, cells were incubated with 50 μg/ml nanobodies and DRAQ7 (0.3 μM—Biostatus) to monitor the permeabilization state. Nanobody binding was detected by using His-tag specific antibody (Invitrogen, catalogue number MA1-135, 1 μg/ml) or Myc-tag specific antibody (Invitrogen, catalogue number 13-2500, 2 μg/ml) and then anti-mouse-Alexa Fluor 488 (Invitrogen, catalogue number A11001, 1.3 μg/ml) at least for 30 min on ice. Cells were washed one time between each step by centrifugation (200×g for 5 min at 4° C.). All incubations (100 μl) and washes (1.5 ml) were performed in PBS with 6% fetal bovine serum (FBS) and 0.01%13-DDM on ice. Cells fluorescence was measured with Gallios Flow Cytometer (Beckman Coulter). Data were analyzed with Kaluza software.

CFTR Pull-Down

Human wt-CFTR was extracted from BHK-21 cells pellet by solubilization with 1% DMNG in PBS with proteases inhibitors for 1 h at 4° C. The cells debris were removed by centrifugation (16,000×g for 30 min at 4° C.). Supernatant was diluted 10 times in PBS with proteases inhibitors plus 10 mM imidazole and incubated at least for 30 min on HisPur Ni-NTA Resin (Thermo Scientific) pre-loaded with nanobodies. Resin was washed with 40 column volumes of PBS with 300 mM NaCl. Nanobodies were eluted with 200 mM imidazole in PBS. Presence of CFTR in each sample was detected by SDS-PAGE and immuno-blotting.

HEK293T Cell Lines Stably Expressing (3HA−) F508del- or WT-CFTR and/or HS-YFP

Stable cell lines were generated by lentiviral transduction as described in (Ensinck, et al. 2020). CFTR variants were cloned to contain a triple hemagglutinin (3HA) tag in the fourth extracellular loop (EC-loop) of CFTR (Sharma, et al. 2004) for maturation and trafficking studies. For HS-YFP quenching studies, double stable cell lines were generated, co-expressing stable HS-YFP (Galietta, et al. 2001).

Immunocytochemistry for PM Detection of 3HA-F508del- or WT-CFTR

For PM staining, cells were blocked with 1% BSA-PBS and incubated with HA.11 antibody (#901515, Biolegend 1:1000) at 4° C. on living cells. Next, cells were fixed (4% PFA) followed by Alexa-488 secondary antibody (#A-11001, Thermo Fischer Scientific, 1:500). Nuclei were stained with DAPI (4′,6-diamidino-2-fenylindole, #D1306, Thermo Fischer Scientific, 1:2000) and sections analyzed by confocal microscopy.

SDS-PAGE and Immunoblotting

Cell extracts were separated by SDS-PAGE on 7.5% polyacrylamide gel and transferred to nitrocellulose membrane (Bio-Rad) for immunodetection. After blocking for 1 h with 5% bovine serum albumin (BSA) in Tris-buffered saline added 0.05% Tween-20 (TBST), CFTR was detected using monoclonal antibody mAb 596, IgG2b (CFTR Antibody Distribution Program, dilution 1:46) for 1 h in blocking buffer. Blot was washed 3 times 5 min and incubated with anti-mouse-HRP antibody (Millipore, catalogue number: AP308P, 0.2 μg/ml) for 1 h in TBST. Membrane was washed 3 times for 5 min. CFTR was visualized by chemiluminescence using Luminata Forte Western HRP Substrate (Millipore) and detected with ImageQuant 400 (GE Healthcare).

ATPase Activity and Functional Stability Assays

To determine effect of nanobodies on functional stability, aliquots of purified wt-CFTR (25 nM) were preincubated 1 h on ice with 1 μM nanobody (or 15 μM, in the case of G3a). Substrate α-[32P]-ATP (2 μl) was then added for measurement of ATPase activity as previously described^{[48]}. Then, nanobody protection against thermal denaturation was determined after a 30 min thermal challenge of the protein complexes followed by an assay of residual ATPase.

NanoDSF

Purified stabilized human CFTR (stab-CFTR: 2PT/ΔRI/R1048A_1172X) was concentrated to 0.5 mg/ml and mixed with 0.1 mg/ml nanobody (^{˜}1:2 molar ratio) and capillaries were loaded with a volume of 10 The capillaries were placed into trays of Prometheus NT.48 (Nanotemper) and subjected to the fluorescence analysis. The emission of fluorescent radiation with the wavelengths of 330 nm and 350 nm was measured with the temperature changes from 25 to 85° C., with the rate of 1° C. min-1. The first derivative of 350 nm fluorescence was used to determine the melting temperature of the proteins.

Statistical Analysis

Affinity constants (KD) and thermodynamic parameters from ITC experiments were determined using one-site binding model with MicroCal Origin 7.0 software (Origin Lab Corp.). Dose response ELISA curves of each Nb binding to either isolated NBD1 or purified FL-CFTR were fitted using the sigmoidal dose-response equation from GraphPad Prism 3. DSC data were analyzed with the MicroCal Origin 7.0 software (Origin Lab Corp.), from which the unfolding temperature (Tm) was obtained.

Halide Sensitive YFP Quenching Assay.

The assay was performed as described previously in (Ensinck, et al. 2020) with minor modifications. Briefly, cells stably expressing wt- or F508del-CFTR and a halide sensitive yellow fluorescent protein (HS-YFP) (Galietta, et al. 2001) were transfected with plasmids encoding the nanobodies using PEI and immediately plated into black, clear-bottomed 96-well plates coated with Poly-D-Lysine. After overnight incubation VX-809 (3 μM) or DMSO was added for 24 h. Next, the cells were washed with DPBS and potentiator VX-770 (3 μM) and/or CFTR activator forskolin (#F3917, Sigma-Aldrich, 10 μM) was added for 20 min. Fluorescence was measured after which a I⁻ buffer (137 mM Nal, 2.7 mM KI, 1.7 mM KH₂PO₄, 10.1 mM Na₂HPO₄, 5 mM D-glucose) was injected into the well and fluorescence monitored for another 4 s. YFP quenching was determined at the end of the interval as F/F₀, and CFTR function as 1-(F/F₀).

Forskolin Induced Swelling (FIS) Assay in Human Intestinal Organoids.

Transduction of human intestinal organoids was performed as described previously (Vidovic, Carlon et al. 2016, Ensinck, De Keersmaecker et al. 2020). Briefly, organoids were trypsinized to single cells, resuspended with equal amounts of viral vector and Matrigel (#356231, Corning) and grown in complete organoid medium (Dekkers, Wiegerinck et al. 2013) containing 10 μM Rock inhibitor (Y-27632-2HCl, #Y0503, Sigma) for the first three days. 14 d post-transduction, FIS was performed as described previously (Dekkers, et al. 2013; Vidovic, et al. 2016; Ensinck, et al. 2020) with minor modifications. VX-809 (3 μM) or DMSO was added to specific wells 24 h before FIS. Organoids were stimulated with forskolin (5 μM) and VX-770 (3 μM) or DMSO, and analyzed by confocal live cell microscopy at 37° C. for 120 min (LSM800, Zeiss, Zen Blue). The total organoid area increase relative to t=0 of forskolin treatment was quantified.

Sequence listing
>SEQ ID NO: 1:
human Cystic fibrosis transmembrane
conductance regulator (CFTR)
(P13569; 1480 aa)
MQRSPLEKASVVSKLFFSWTRPILRKGYRQRLELS
DIYQIPSVDSADNLSEKLEREWDRELASKKNPKLI
NALRRCFFWRFMFYGIFLYLGEVTKAVQPLLLGRI
IASYDPDNKEERSIAIYLGIGLCLLFIVRTLLLHP
AIFGLHHIGMQMRIAMFSLIYKKTLKLSSRVLDKI
SIGQLVSLLSNNLNKFDEGLALAHFVWIAPLQVAL
LMGLIWELLQASAFCGLGFLIVLALFQAGLGRMMM
KYRDQRAGKISERLVITSEMIENIQSVKAYCWEEA
MEKMIENLRQTELKLTRKAAYVRYFNSSAFFFSGF
FVVFLSVLPYALIKGIILRKIFTTISFCIVLRMAV
TRQFPWAVQTWYDSLGAINKIQDFLQKQEYKTLEY
NLTTTEVVMENVTAFWEEGFGELFEKAKQNNNNRK
TSNGDDSLFFSNFSLLGTPVLKDINFKIERGQLLA
VAGSTGAGKTSLLMVIMGELEPSEGKIKHSGRISF
CSQFSWIMPGTIKENIIFGVSYDEYRYRSVIKACQ
LEEDISKFAEKDNIVLGEGGITLSGGQRARISLAR
AVYKDADLYLLDSPFGYLDVLTEKEIFESCVCKLM
ANKTRILVTSKMEHLKKADKILILHEGSSYFYGTF
SELQNLQPDFSSKLMGCDSFDQFSAERRNSILTET
LHRFSLEGDAPVSWTETKKQSFKQTGEFGEKRKNS
ILNPINSIRKFSIVQKTPLQMNGIEEDSDEPLERR
LSLVPDSEQGEAILPRISVISTGPTLQARRRQSVL
NLMTHSVNQGQNIHRKTTASTRKVSLAPQANLTEL
DIYSRRLSQETGLEISEEINEEDLKECFFDDMESI
PAVTTWNTYLRYITVHKSLIFVLIWCLVIFLAEVA
ASLVVLWLLGNTPLQDKGNSTHSRNNSYAVIITST
SSYYVFYIYVGVADTLLAMGFFRGLPLVHTLITVS
KILHHKMLHSVLQAPMSTLNTLKAGGILNRFSKDI
AILDDLLPLTIFDFIQLLLIVIGAIAVVAVLQPYI
FVATVPVIVAFIMLRAYFLQTSQQLKQLESEGRSP
IFTHLVTSLKGLWTLRAFGRQPYFETLFHKALNLH
TANWFLYLSTLRWFQMRIEMIFVIFFIAVTFISIL
TTGEGEGRVGIILTLAMNIMSTLQWAVNSSIDVDS
PLMRSVSRVFKFIDMPTEGKTKSTKPYKNGQLSKV
MMIIENSHVKKDDIWPSGGQTVKDLTAKYTEGGNA
ILENISFSISPGQRVGLLGRTGSGKSTLLSAFLRL
LNTEGEIQIDGVSWDSITLQQWRKAFGVIPQKVFI
FSGTFRKNLDPYEQWSDQEIWKVADEVGLRSVIEQ
FPGKLDFVLVDGGCVLSHGHKQLMCLARSVLSKAK
ILLLDEPSAHLDPVTYQIIRRTLKQAFADCTVILC
KEHRIEAMLECQQFLVIEENVRQYDSIQKLLNERS
LFRQAISPSDRVKLFPHRNSSKCKSKPQIAALKEE
TEEEVQDTRL
>SEQ ID NO: 2:
D12 Nanobody amino acid sequence
(123aa)
QVQLQESGGGLVQAGSSLRLACAATGSIRSINNMG
WYRQAPGKQRGMVAIITRVGNTDYADSVKGRFTIS
RDNAKNTVYLQMNSLKPEDTATYYCHAEITEQSRP
FYLTDDYWGQGTQVTVSS
>SEQ ID NO: 3:
T2a Nanobody amino acid sequence
(120aa)
QVQLQESGGGLVQAGGSLRLSCAASGSIFRIDAMG
WYRQAPGKQRELVAHSTSGGSTDYADSVKGRFTIS
RDNAKNTVYLQMNSLKPEDTAVYYCNADVRTRWYA
SNNYWGQGTQVTVSS
>SEQ ID NO: 4:
T27 Nanobody amino acid sequence
(123aa)
QVQLQESGGGLEQPGGSLRLSCATSGVIFGINAMG
WYRQAPGKQRELVATFTSGGSTNYADFVEGRFTIS
RDNAKNTVYLQMNGLRPEDTAVYYCHATVVVSRYG
LTYDYWGQGTQVTVSS
>SEQ ID NO: 5:
G5 Nanobody amino acid sequence
(123aa)
QVQLQESGGGLVQAGGSLRLACAATGSIRNINTMG
WYRQAPGKQRDMVAFITRAGNTDYADSVKGRFTIS
RDNARNTVYLRMNSLKPEDTATYYCHAEIAERSRP
FYLTDDYWGQGTQVTVSS
>SEQ ID NO: 6:
T4 Nanobody amino acid sequence
(117aa)
QVQLQESGGGLVQAGGSLRLSCAASGSTFAIIAMG
WYRQAPGKQRELVAVISTGDTRYADSVKGRFTISR
DNAKNTVYLQMDSLRPEDTAVYYCNAAVQVRDYRN
YWGQGTQVTVSS
>SEQ ID NO: 7:
T8 Nanobody amino acid sequence
(117aa)
QVQLQESGGGLVQPGGSLRLSCAASGSTSSINAMG
WYRQAPGKQREPVAISSSGGDTRYAEPVKGRFTIS
RDNAQNKVYLQMNSLKPEDTAVYYCWLNWGRTSVN
SWGQGTQVTVSS

TABLE 2
Nanobody CDR and FR regions (as defined in FIG. 8 and as provided in the sequence list).

| | SEQ ID NO | SEQ ID NO | SEQ ID NO | SEQ ID NO | SEQ ID NO | SEQ ID NO | SEQ ID NO |
|---|---|---|---|---|---|---|---|
| Nb | of FR1 | of CDR1 | of FR2 | of CDR2 | of FR3 | of CDR3 | of FR4 |
| D12 | 8 | 9 | 10 | 11 | 12 | 13 | 14 |
| T2a | 15 | 16 | 17 | 18 | 19 | 20 | 21 |
| T27 | 22 | 23 | 24 | 25 | 26 | 27 | 28 |
| G5 | 29 | 30 | 31 | 32 | 33 | 34 | 35 |
| T4 | 36 | 37 | 38 | 39 | 40 | 41 | 42 |
| T8 | 43 | 44 | 45 | 46 | 47 | 48 | 49 |
| G3a | 51 | 52 | 53 | 54 | 55 | 56 | 57 |

>SEQ ID NO: 29: G5 FR1
QVQLQESGGGLVQAGGSLRLACAAT
>SEQ ID NO: 30: G5 CDR1
GSIRNINT
>SEQ ID NO: 31: G5 FR2
MGWYRQAPGKQRDMVAF
>SEQ ID NO: 32: G5 CDR2
ITRAGNTD
>SEQ ID NO: 33: G5 FR3
YADSVKGRFTISRDNARNTVYLRMNS
LKPEDTATYYC
>SEQ ID NO: 34: G5 CDR3
HAEIAERSRPFYLTDD
>SEQ ID NO: 35: G5 FR4
YWGQGTQVTVSS
>SEQ ID NO: 50:
G3a Nanobody amino acid sequence
(123aa)
QVQLQESGGGLVQAGGSLRLSCTASGRAFSWYV
MGWFRQAPGKEREFVATVSGNGSRRDYADSVKG
RFTISRDNAKNTVYLQMNSLKPEDTAVYYCAAS
STYYYTDPEKYDYWGQGTQVTVSS
>SEQ ID NO: 58:
2PT-NBD1 amino acid sequence (NBD1
residues 387-646 from WT-NBD1
construct from ASU (clone id:
287401), containing the mutations
S492P, A534P, I539T; 259aa)
TTTEVVMENVTAFWEEGFGELFEKAKQNNNNRKTS
NGDDSLFFSNFSLLGTPVLKDINFKIERGQLLAVA
GSTGAGKTSLLMMIMGELEPSEGKIKHSGRISFCP
QFSWIMPGTIKENIIFGVSYDEYRYRSVIKACQLE
EDISKFPEKDNTVLGEGGITLSGGQRARISLARAV
YKDADLYLLDSPFGYLDVLTEKEIFESCVCKLMAN
KTRILVTSKMEHLKKADKILILHEGSSYFYGTFSE
LQNLQPDFSSKLMG
>SEQ ID NO: 59:
human ARI-NBD1 amino acid sequence
(residues 387-646, A405-436 of
CFTR; 227 aa)
TTTEVVMENVTAFWEEGGTPVLKDINFKIERGQLL
AVAGSTGAGKTSLLMMIMGELEPSEGKIKHSGRIS
FCSQFSWIMPGTIKENIIFGVSYDEYRYRSVIKAC
QLEEDISKFAEKDNIVLGEGGITLSGGQRARISLA
RAVYKDADLYLLDSPFGYLDVLTEKEIFESCVCKL
MANKTRILVTSKMEHLKKADKILILHEGSSYFYGT
FSELQNLQPDFSSKLMG
>SEQ ID NO: 60:
C-terminal cleavage site for
human rhinovirus 3C (P3C)
LEVLFQGP
>SEQ ID NO: 61:
cMyc tag
EQKLISEEDL
>SEQ ID NO: 62:
Twin-Strep-tag
WSHPQFEKGGGSGGGSGGSAWSHPQFEK
>SEQ ID NO: 63:
T5 Nanobody amino acid sequence
(121 aa)
QVQLQESGGGLVQAGGSLRLACVASGNIFTINDMG
WYRQAPGKQRELVATITSAGITNYADSVKGRFTIS
RDNAKNTVFLRMISLKPEDTAVYYCHQAVVHGPIG
LEYDYWGQGTQVTVSS
>SEQ ID NO: 64:
51 Nanobody amino acid sequence
(127 aa)
QVQLQESGGGLVQPGGSLRLSCATSRFTLDYGTIG
WFRQAPGKEREGVSCIRTSSGSTNYADSVKGRFTI
YRDIVKNTIYLQMNSLKPEDTAAYYCAADEARLYG
SSCLRMDEYDYWGQGTQVTVSS
>SEQ ID NO: 65:
T29-del Nanobody amino acid
sequence (122aa)
QVQLQESGGGLVQPGDSLRLSCAASGFTMGNYAIG
WFRQAPGKEREGIACIGTSAGITNYADSVKGRFTI
SRDNAKNTVFLRMISLKPEDTAVYYCHQAVVHGPI
GLEYDYWGQGTQVTVSS
>SEQ ID NO: 66:
T29 Nanobody amino acid sequence
(198aa)
QVQLQESGGGLVQPGDSLRLSCAASGFTMGNYAIG
WFRQAPGKEREGIACIGANDGKTYYSDSVKGRFAA
SRDNAKSVAYLQESGGGLVQAGGSLRLACVASGNI
FTINNMGWYRQAPGKQRELVAFITSAGITNYADSV
KGRFTISRDNAKNTVFLRMISLKPEDTAVYYCHQA
VVHGPIGLEYDYWGQGTQVTVSS
>SEQ ID NO: 67:
T2a mutant A105F (120 aa)
QVQLQESGGGLVQAGGSLRLSCAASGSIFRIDAMG
WYRQAPGKQRELVAHSTSGGSTDYADSVKGRFTIS
RDNAKNTVYLQMNSLKPEDTAVYYCNADVRTRWYF
SNNYWGQGTQVTVSS

### REFERENCES

1. Riordan, J. et al. Identification of the cystic fibrosis gene: cloning and characterization of complementary DNA. Science 245, 1066-1073 (1989).
2. Liu, F. et al. Molecular Structure of the Human CFTR Ion Channel. Cell 169, 85-95.e8 (2017).
3. Zhang, Z. & Chen, J. Atomic Structure of the Cystic Fibrosis Transmembrane Conductance Regulator. Cell 167, 1586-1597 (2016).
4. Zhang, Z., Liu, F. & Chen, J. Conformational Changes of CFTR upon Phosphorylation and ATP Binding. Cell 170, 483-491.e8 (2017).
5. Lewis, H. A. et al. Structure of nucleotide-binding domain 1 of the cystic fibrosis transmembrane conductance regulator. EMBO J. 23, 282-293 (2004).
6. Lewis, H. A. et al. Impact of the deltaF508 mutation in first nucleotide-binding domain of human cystic fibrosis transmembrane conductance regulator on domain folding and structure. Journal of Biological Chemistry 280, 1346-1353 (2005).
7. Atwell, S. et al. Structures of a minimal human CFTR first nucleotide-binding domain as a monomer, head-to-tail homodimer, and pathogenic mutant. Protein Eng. Des. Sel. 23, 375-384 (2010).
8. Riordan, J. R. CFTR function and prospects for therapy. Annu. Rev. Biochem. 77, 701-726 (2008).
9. Aleksandrov, A. A. et al. Regulatory insertion removal restores maturation, stability and function of DeltaF508 CFTR. Journal of Molecular Biology 401, 194-210 (2010).
10. Pankow, S. et al. ΔF508 CFTR interactome remodelling promotes rescue of cystic fibrosis. Nature 528, 1-18 (2015).
11. Protasevich, I. et al. Thermal unfolding studies show the disease causing F508del mutation in CFTR thermodynamically destabilizes nucleotide-binding domain 1. Protein Sci. 19, 1917-1931 (2010).
12. Okiyoneda, T. et al. Mechanism-based corrector combination restores ΔF508-CFTR folding and function. Nat. Chem. Biol. 9, 444-454 (2013).
13. He, L. et al. Restoration of domain folding and interdomain assembly by second-site suppressors of the ΔF508 mutation in CFTR. The FASEB Journal 24, 3103-3112 (2010).
14. He, L. et al. Restoration of NBD1 thermal stability is necessary and sufficient to correct dF508 CFTR folding and assembly. J. Mol. Biol. 427, 106-120 (2015).
15. He, L. et al. Correctors of ΔF508 CFTR restore global conformational maturation without thermally stabilizing the mutant protein. FASEB Journal 27, 536-545 (2013).
16. Pardon, E. et al. A general protocol for the generation of Nanobodies for structural biology. Nat. Protoc. 9, 674-93 (2014).
17. Manglik, A., Kobilka, B. K. & Steyaert, J. Nanobodies to Study G Protein-Coupled Receptor Structure and Function. Annu. Rev. Pharmacol. Toxicol. 57, 19-37 (2017).
18. Ward, A. B. et al. Structures of P-glycoprotein reveal its conformational flexibility and an epitope on the nucleotide-binding domain. Proceedings of the National Academy of Sciences 110, 13386-91 (2013).
19. Aleksandrov, A. A. et al. Allosteric modulation balances thermodynamic stability and restores function of Deltaf508 CFTR. Journal of Molecular Biology 419, 41-60 (2012).
20. De Genst, E., Saerens, D., Muyldermans, S. & Conrath, K. Antibody repertoire development in camelids. Dev. Comp. Immunol. 30, 187-198 (2006).
21. Teem, J. L. et al. Identification of revertants for the cystic fibrosis DeltaF508 mutation using STE6-CFTR chimeras in yeast. Cell 73, 335-346 (1993).
22. Rabeh, W. M. et al. Correction of both NBD1 energetics and domain interface is required to restore df508 CFTR folding and function. Cell 148, 150-163 (2012).
23. Dong, A., Xu, X. & Edwards, A. In situ proteolysis for protein crystallization and structure determination. Nat Methods 4, 1019-1021 (2007).
24. Desmyter, A. et al. Crystal structure of a camel single-domain V(H) antibody fragment in complex with lysozyme. Nat. Struct. Biol. 3, 803-811 (1996).
25. Lewis, H. A. et al. Structure and dynamics of NBD1 from CFTR characterized using crystallography and hydrogen/deuterium exchange mass spectrometry. J. Mol. Biol. 396, 406-430 (2010).
26. Hall, J. D. et al. Binding screen for cftr correctors finds new chemical matter and yields insights into cf therapeutic strategy. Protein Sci. 22, 360-373 (2015).
27. Kanelis, V., Hudson, R. P., Thibodeau, P. H., Thomas, P. J. & Forman-Kay, J. D. NMR evidence for differential phosphorylation-dependent interactions in WT and ΔF508 CFTR. EMBO J. 29, 263-277 (2010).
28. Yang, Z. et al. Structural stability of purified human CFTR is systematically improved by mutations in nucleotide binding domain 1. Biochim. Biophys. Acta—Biomembr. 1860, 1193-1204 (2018).
29. Pissarra, L. S. et al. Solubilizing Mutations Used to Crystallize One CFTR Domain Attenuate the Trafficking and Channel Defects Caused by the Major Cystic Fibrosis Mutation. Chem. Biol. 15, 62-69 (2008).
30. Steeland, S., Vandenbroucke, R. E. & Libert, C. Nanobodies as therapeutics: Big opportunities for small antibodies. Drug Discovery Today 21, 1076-1113 (2016).
31. Staus, D. P. et al. Regulation of 2-Adrenergic Receptor Function by Conformationally Selective Single-Domain Intrabodies. Mol. Pharmacol. 85, 472-481 (2014).
32. Weill, C. O., Biri, S., Adib, A. & Erbacher, P. A practical approach for intracellular protein delivery. Cytotechnology 56, 41-48 (2008).
33. D'Astolfo, D. S. et al. Efficient intracellular delivery of native proteins. Cell 161, 674-690 (2015).
34. Serohijos, A. W. R. et al. Phenylalanine-508 mediates a cytoplasmic-membrane domain contact in the CFTR 3D structure crucial to assembly and channel function. Proc. Natl. Acad. Sci. 105, 3256-3261 (2008).
35. Mehmood, S., Domene, C., Forest, E. & Jault, J.-M. Dynamics of a bacterial multidrug ABC transporter in the inward- and outward-facing conformations. Proc. Natl. Acad. Sci. U.S.A. 109, 10832-10836 (2012).
36. Li, S. J. & Hochstrasser, M. A new protease required for cell-cycle progression in yeast. Nature 398, 246-251 (1999).
37. Kabsch, W. XDS. Acta Crystallogr. Sect. D Biol. Crystallogr. 66, 125-132 (2010).
38. Vagin, A. & Teplyakov, A. MOLREP: an Automated Program for Molecular Replacement. J. Appl. Crystallogr. 30, 1022-1025 (1997).
39. Emsley, P., Lohkamp, B., Scott, W. G. & Cowtan, K. Features and development of Coot. Acta Crystallogr. Sect. D Biol. Crystallogr. 66, 486-501 (2010).
40. Bricogne, G. et al. Bricogne, BUSTER version 2.10.1, Cambridge, United Kingdom: Global Phasing Ltd. (2017).
41. Chen, V. B. et al. MolProbity: All-atom structure validation for macromolecular crystallography. Acta Crystallogr. Sect. D Biol. Crystallogr. 66, 12-21 (2010).
42. Pettersen, E. F. et al. UCSF Chimera—a visualization system for exploratory research and analysis. J. Comput. Chem. 25, 1605-1612 (2004).
43. Chang, X. B. et al. Protein kinase a (PKA) still activates CFTR chloride channel after mutagenesis of all 10 PKA consensus phosphorylation sites. J. Biol. Chem. 268, 11304-11311 (1993).
44. He, L. et al. Multiple membrane-cytoplasmic domain contacts in the cystic fibrosis transmembrane conductance regulator (CFTR) mediate regulation of channel gating. J. Biol. Chem. 283, 26383-26390 (2008).
45. Hildebrandt, E. et al. A Stable Human-Cell System Overexpressing Cystic Fibrosis Transmembrane Conductance Regulator Recombinant Protein at the Cell Surface. Mol. Biotechnol. 57, 391-405 (2015).
46. Cui, L. et al. Domain Interdependence in the Biosynthetic Assembly of CFTR. Journal of Molecular Biology 365, 981-994 (2007).
47. Grimard, V. et al. Phosphorylation-induced Conformational Changes of Cystic Fibrosis Transmembrane Conductance Regulator Monitored by Attenuated Total Reflection-Fourier Transform IR Spectroscopy and Fluorescence Spectroscopy. Journal of Biological Chemistry 279, 5528-5536 (2004).
48. Hildebrandt, E. et al. Specific stabilization of CFTR by phosphatidylserine. Biochim. Biophys. Acta-Biomembr. 1859, 289-293 (2017).
49. Kunzelmann, K., et al. Control of epithelial Na+ conductance by the cystic fibrosis transmembrane conductance regulator. Pflugers Arch. 440(2):193-201 (2000).
50. Mathee, K., et al. Mucoid conversion of Pseudomonas aeruginosa by hydrogen peroxide: a mechanism for virulence activation in the cystic fibrosis lung. Microbiology. 145 (Pt 6):1349-57 (1999).
51. Choo-Kang L R, Zeitlin P L. Type I, II, III, IV, and V cystic fibrosis transmembrane conductance regulator defects and opportunities for therapy. Curr Opin Pulm Med, 6:521-529 (2000).
52. Ikpa, P. T., et al. Cystic fibrosis: Toward personalized therapies. Intl. J. Biochem. & Cell Biol. 52: 192-200 (2014).
53. Dekkers, J. F., C. L. Wiegerinck, H. R. de Jonge, I. Bronsveld, H. M. Janssens, K. M. de Winter-de Groot, A. M. Brandsma, N. W. de Jong, M. J. Bijvelds, B. J. Scholte, E. E. Nieuwenhuis, S. van den Brink, H. Clevers, C. K. van der Ent, S. Middendorp and J. M. Beekman (2013). “A functional CFTR assay using primary cystic fibrosis intestinal organoids.” Nat Med 19(7): 939-945.
54. Ensinck, M., L. De Keersmaecker, L. Heylen, A. S. Ramalho, R. Gijsbers, R. Farre, K. De Boeck, F. Christ, Z. Debyser and M. S. Carlon (2020). “Phenotyping of Rare CFTR Mutations Reveals Distinct Trafficking and Functional Defects.” Cells 9(3).
55. Galietta, L. J., P. M. Haggie and A. S. Verkman (2001). “Green fluorescent protein-based halide indicators with improved chloride and iodide affinities.” FEBS Lett 499(3): 220-224.
56. Sharma, M., F. Pampinella, C. Nemes, M. Benharouga, J. So, K. Du, K. G. Bache, B. Papsin, N. Zerangue, H. Stenmark and G. L. Lukacs (2004). “Misfolding diverts CFTR from recycling to degradation: quality control at early endosomes.” J Cell Biol 164(6): 923-933.
57. Vidovic, D., M. S. Carlon, M. F. da Cunha, J. F. Dekkers, M. I. Hollenhorst, M. J. Bijvelds, A. S. Ramalho, C. Van den Haute, M. Ferrante, V. Baekelandt, H. M. Janssens, K. De Boeck, I. Sermet-Gaudelus, H. R. de Jonge, R. Gijsbers, J. M. Beekman, A. Edelman and Z. Debyser (2016). “rAAV-CFTRDeltaR Rescues the Cystic Fibrosis Phenotype in Human Intestinal Organoids and Cystic Fibrosis Mice.” Am J Respir Crit Care Med 193(3): 288-298.

1.-17. (canceled)

18. A polypeptide, wherein the polypeptide is an antibody, antibody mimetic, ISVD, or active antibody fragment, which specifically binds Cystic Fibrosis Transmembrane Conductance Regulator (CFTR), and upon binding to CFTR, increases the thermal stability of CFTR by at least 5° C. as compared to non-bound CFTR under the same conditions.”

19. A polypeptide comprising a sequence selected from the group consisting of SEQ ID NO: 9, 11, 13, 16, 18, 20, 23, 25, 27, 30, 32, 34, 37, 39, 41, 44, 46, and 48.

20. The polypeptide of claim 19, wherein the polypeptide comprises:
a sequence selected from the group consisting of SEQ ID NO: 9, 16, 23, 30, 37, 44;
a sequence selected from the group consisting of SEQ ID NO: 11, 18, 25, 32, 39, 46; and
a sequence selected from the group consisting of SEQ ID NO: 13, 20, 27, 34, 41, 48.

21. The polypeptide of claim 20, wherein the polypeptide comprises an antibody, an antibody mimetic, an immunoglobulin single variable domain (ISVD), or an active antibody fragment,
wherein the antibody, antibody mimetic, ISVD, or active antibody fragment comprises 4 framework regions (FR), and 3 complementarity determining regions (CDR) according to the following formula (1): FR1-CDR1-FR2-CDR2-FR3-CDR3-FR4 (1);
wherein CDR1 is selected from the group consisting of SEQ ID NO: 9, 16, 23, 30, 37, 44; CDR2 is selected from the group consisting of SEQ ID NO: 11, 18, 25, 32, 39, 46; and CDR3 is selected from the group consisting of SEQ ID NO: 13, 20, 27, 34, 41, 48; and
wherein the antibody, antibody mimetic, ISVD, or active antibody fragment specifically binds the Cystic Fibrosis Transmembrane Conductance Regulator (CFTR).

22. The polypeptide of claim 21, wherein the antibody, antibody mimetic, ISVD, or active antibody fragment, upon binding, increases the thermal stability of CFTR by at least 5° C. as compared to non-bound CFTR under the same conditions.

23. The polypeptide of claim 22, wherein the binding site on the CFTR comprises amino acid residues 457, 459, 550-551, 576-581, 605-608, 610, 618, 625, and 633 of SEQ ID NO:1, or comprises amino acid residues 472, 474, 490, 494-499, 508-510, 560, and 564 of SEQ ID NO:1.

24. The polypeptide of claim 21, wherein the polypeptide comprises a sequence selected from the group consisting of SEQ ID NOs: 2 to 7, or a sequence with at least 90% amino acid identity to SEQ ID NOs: 2-7, or a humanized variant of anyone thereof.

25. The polypeptide of claim 21, wherein the antibody, antibody mimetic, ISVD, or active antibody fragment is coupled via a linker or spacer to a binding agent.

26. The polypeptide of claim 25, wherein the polypeptide is a bispecific binding agent and wherein the binding site of the binding agent is different that the binding site of the antibody, antibody mimetic, ISVD, or active antibody fragment.

27. The polypeptide of claim 19, wherein the polypeptide is comprised in a composition.

28. The polypeptide of claim 27, wherein the composition further comprises a small molecule compound, wherein the small molecule compound is a Cystic Fibrosis Transmembrane Conductance Regulator (CFTR) corrector and/or a CFTR potentiator.

29. A complex comprising the polypeptide of claim 21 and Cystic Fibrosis Transmembrane Conductance Regulator Nucleotide-Binding Domain 1 (CFTR NDB1).

30. The complex of claim 28, wherein the complex is crystalline.

31. The complex of claim 29, wherein the CFTR NBD1 is a domain with an amino acid sequence with at least 90% identity to SEQ ID NO:58 or SEQ ID NO:59, and characterized in that the crystal is:
i) a crystal between the CFTR NBD1 domain and said binding agent in the space group C121, with the following crystal lattice constants: a=152.2 Å±5%, b=41.6 Å±5%, c=99.3 Å±5%, α=90°, β=120.56°, γ=90°, or
ii) a crystal between the CFTR NBD1 domain and the antibody, antibody mimetic, ISVD, or active antibody fragment in the space group C222₁, with the following crystal lattice constants: a=38.68 Å±5%, b=135.78 Å±5%, c=190.65 Å±5%, α=β=γ=90°, or
iii) a crystal between the CFTRNBD1 domain, and the antibody, antibody mimetic, ISVD, or active antibody fragment in the space group P212121, with the following crystal lattice constants: a=64.49 Å±5%, b=118.15 Å±5%, c=180.21 Å±5%, α=β=γ=90°, or
iv) a crystal between the CFTRNBD1 domain, and the antibody, antibody mimetic, ISVD, or active antibody fragment in the space group P1211, with the following crystal lattice constants: a=80.94 Å±5%, b=55.19 Å±5%, c=114.99 Å±5%, α=90°, β=103.96°, γ=90°.

32. The complex of claim 30, wherein the crystal has a three-dimensional structure wherein the crystal i) comprises an atomic structure characterized by the coordinates of PDB: 6GJS, or wherein the crystal ii) comprises an atomic structure characterized by the coordinates of PDB: 6GJU or a subset of atomic coordinates thereof, or wherein the crystal iii) comprises an atomic structure characterized by the coordinates of PDB: 6GJQ or a subset of atomic coordinates thereof, or wherein the crystal iv) comprises an atomic structure characterized by the coordinates of PDB: 6GK4 or a subset of atomic coordinates thereof.

33. A method of treating cystic fibrosis, the method comprising administering to a subject in need thereof the polypeptide of claim 21.


SEQUENCE LISTING (67 sequences)

SEQ ID NO: 1 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 2 - protein, 123 aa, Artificial Sequence
  D12 Nanobody amino acid sequence
QVQLQESGGGLVQAGSSLRLACAATGSIRSINNMGWYRQAPGKQRGMVAIITRVGNTDYADSVKGRFTISRDNAKNTVYLQMNSLKPEDTATYYCHAEITEQSRPFYLTDDYWGQGTQVTVSS

SEQ ID NO: 3 - protein, 120 aa, Artificial Sequence
  T2a Nanobody amino acid sequence
QVQLQESGGGLVQAGGSLRLSCAASGSIFRIDAMGWYRQAPGKQRELVAHSTSGGSTDYADSVKGRFTISRDNAKNTVYLQMNSLKPEDTAVYYCNADVRTRWYASNNYWGQGTQVTVSS

SEQ ID NO: 4 - protein, 121 aa, Artificial Sequence
  T27 Nanobody amino acid sequence
QVQLQESGGGLEQPGGSLRLSCATSGVIFGINAMGWYRQAPGKQRELVATFTSGGSTNYADFVEGRFTISRDNAKNTVYLQMNGLRPEDTAVYYCHATVVVSRYGLTYDYWGQGTQVTVSS

SEQ ID NO: 5 - protein, 123 aa, Artificial Sequence
  G5 Nanobody amino acid sequence
QVQLQESGGGLVQAGGSLRLACAATGSIRNINTMGWYRQAPGKQRDMVAFITRAGNTDYADSVKGRFTISRDNARNTVYLRMNSLKPEDTATYYCHAEIAERSRPFYLTDDYWGQGTQVTVSS

SEQ ID NO: 6 - protein, 117 aa, Artificial Sequence
  T4 Nanobody amino acid sequence
QVQLQESGGGLVQAGGSLRLSCAASGSTFAIIAMGWYRQAPGKQRELVAVISTGDTRYADSVKGRFTISRDNAKNTVYLQMDSLRPEDTAVYYCNAAVQVRDYRNYWGQGTQVTVSS

SEQ ID NO: 7 - protein, 117 aa, Artificial Sequence
  T8 Nanobody amino acid sequence
QVQLQESGGGLVQPGGSLRLSCAASGSTSSINAMGWYRQAPGKQREPVAISSSGGDTRYAEPVKGRFTISRDNAQNKVYLQMNSLKPEDTAVYYCWLNWGRTSVNSWGQGTQVTVSS

SEQ ID NO: 8 - protein, 25 aa, Artificial Sequence
  FR1 D12
QVQLQESGGGLVQAGSSLRLACAAT

SEQ ID NO: 9 - protein, 8 aa, Artificial Sequence
  CDR1 D12
GSIRSINN

SEQ ID NO: 10 - protein, 17 aa, Artificial Sequence
  FR2 D12
MGWYRQAPGKQRGMVAI

SEQ ID NO: 11 - protein, 8 aa, Artificial Sequence
  CDR2 D12
ITRVGNTD

SEQ ID NO: 12 - protein, 37 aa, Artificial Sequence
  FR3 D12
YADSVKGRFTISRDNAKNTVYLQMNSLKPEDTATYYC

SEQ ID NO: 13 - protein, 16 aa, Artificial Sequence
  CDR3 D12
HAEITEQSRPFYLTDD

SEQ ID NO: 14 - protein, 15 aa, Artificial Sequence
  FR4 D12
YWGQGTQVTVSSAAA

SEQ ID NO: 15 - protein, 25 aa, Artificial Sequence
  FR1 T2a
QVQLQESGGGLVQAGGSLRLSCAAS

SEQ ID NO: 16 - protein, 8 aa, Artificial Sequence
  CDR1 T2a
GSIFRIDA

SEQ ID NO: 17 - protein, 17 aa, Artificial Sequence
  FR2 T2a
MGWYRQAPGKQRELVAH

SEQ ID NO: 18 - protein, 8 aa, Artificial Sequence
  CDR2 T2a
STSGGSTD

SEQ ID NO: 19 - protein, 37 aa, Artificial Sequence
  FR3 T2a
YADSVKGRFTISRDNAKNTVYLQMNSLKPEDTAVYYC

SEQ ID NO: 20 - protein, 13 aa, Artificial Sequence
  CDR3 T2a
NADVRTRWYASNN

SEQ ID NO: 21 - protein, 15 aa, Artificial Sequence
  FR4 T2a
YWGQGTQVTVSSAAA

SEQ ID NO: 22 - protein, 25 aa, Artificial Sequence
  FR1 T27
QVQLQESGGGLEQPGGSLRLSCATS

SEQ ID NO: 23 - protein, 8 aa, Artificial Sequence
  CDR1 T27
GVIFGINA

SEQ ID NO: 24 - protein, 17 aa, Artificial Sequence
  FR2 T27
MGWYRQAPGKQRELVAT

SEQ ID NO: 25 - protein, 8 aa, Artificial Sequence
  CDR2 T27
FTSGGSTN

SEQ ID NO: 26 - protein, 37 aa, Artificial Sequence
  FR3 T27
YADFVEGRFTISRDNAKNTVYLQMNGLRPEDTAVYYC

SEQ ID NO: 27 - protein, 14 aa, Artificial Sequence
  CDR3 T27
HATVVVSRYGLTYD

SEQ ID NO: 28 - protein, 15 aa, Artificial Sequence
  FR4 T27
YWGQGTQVTVSSAAA

SEQ ID NO: 29 - protein, 25 aa, Artificial Sequence
  FR1 G5
QVQLQESGGGLVQAGGSLRLACAAT

SEQ ID NO: 30 - protein, 8 aa, Artificial Sequence
  CDR1 G5
GSIRNINT

SEQ ID NO: 31 - protein, 17 aa, Artificial Sequence
  FR2 G5
MGWYRQAPGKQRDMVAF

SEQ ID NO: 32 - protein, 8 aa, Artificial Sequence
  CDR2 G5
ITRAGNTD

SEQ ID NO: 33 - protein, 37 aa, Artificial Sequence
  FR3 G5
YADSVKGRFTISRDNARNTVYLRMNSLKPEDTATYYC

SEQ ID NO: 34 - protein, 16 aa, Artificial Sequence
  CDR3 G5
HAEIAERSRPFYLTDD

SEQ ID NO: 35 - protein, 12 aa, Artificial Sequence
  FR4 G5
YWGQGTQVTVSS

SEQ ID NO: 36 - protein, 25 aa, Artificial Sequence
  FR1 T4
QVQLQESGGGLVQAGGSLRLSCAAS

SEQ ID NO: 37 - protein, 8 aa, Artificial Sequence
  CDR1 T4
GSTFAIIA

SEQ ID NO: 38 - protein, 17 aa, Artificial Sequence
  FR2 T4
MGWYRQAPGKQRELVAV

SEQ ID NO: 39 - protein, 7 aa, Artificial Sequence
  CDR2 T4
ISTGDTR

SEQ ID NO: 40 - protein, 37 aa, Artificial Sequence
  FR3 T4
YADSVKGRFTISRDNAKNTVYLQMDSLRPEDTAVYYC

SEQ ID NO: 41 - protein, 11 aa, Artificial Sequence
  CDR3 T4
NAAVQVRDYRN

SEQ ID NO: 42 - protein, 15 aa, Artificial Sequence
  FR4 T4
YWGQGTQVTVSSAAA

SEQ ID NO: 43 - protein, 25 aa, Artificial Sequence
  FR1 T8
QVQLQESGGGLVQPGGSLRLSCAAS

SEQ ID NO: 44 - protein, 8 aa, Artificial Sequence
  CDR1 T8
GSTSSINA

SEQ ID NO: 45 - protein, 17 aa, Artificial Sequence
  FR2 T8
MGWYRQAPGKQREPVAI

SEQ ID NO: 46 - protein, 8 aa, Artificial Sequence
  CDR2 T8
SSSGGDTR

SEQ ID NO: 47 - protein, 37 aa, Artificial Sequence
  FR3 T8
YAEPVKGRFTISRDNAQNKVYLQMNSLKPEDTAVYYC

SEQ ID NO: 48 - protein, 10 aa, Artificial Sequence
  CDR3 T8
WLNWGRTSVN

SEQ ID NO: 49 - protein, 15 aa, Artificial Sequence
  FR4 T8
SWGQGTQVTVSSAAA

SEQ ID NO: 50 - protein, 123 aa, Artificial Sequence
  G3a Nanobody
QVQLQESGGGLVQAGGSLRLSCTASGRAFSWYVMGWFRQAPGKEREFVATVSGNGSRRDYADSVKGRFTISRDNAKNTVYLQMNSLKPEDTAVYYCAASSTYYYTDPEKYDYWGQGTQVTVSS

SEQ ID NO: 51 - protein, 25 aa, Artificial Sequence
  FR1 G3a
QVQLQESGGGLVQAGGSLRLSCTAS

SEQ ID NO: 52 - protein, 8 aa, Artificial Sequence
  CDR1 G3a
GRAFSWYV

SEQ ID NO: 53 - protein, 17 aa, Artificial Sequence
  FR2 G3a
MGWFRQAPGKEREFVAT

SEQ ID NO: 54 - protein, 9 aa, Artificial Sequence
  CDR2 G3a
VSGNGSRRD

SEQ ID NO: 55 - protein, 37 aa, Artificial Sequence; identical to SEQ ID NO: 19
  FR3 G3a

SEQ ID NO: 56 - protein, 15 aa, Artificial Sequence
  CDR3 G3a
AASSTYYYTDPEKYD

SEQ ID NO: 57 - protein, 15 aa, Artificial Sequence
  FR4 G3a
YWGQGTQVTVSSAAA

SEQ ID NO: 58 - protein, 259 aa, Artificial Sequence
  2PT-NBD1
TTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCPQFSWIMPGTIKENIIFGVSYDEYRYRSVIKACQLEEDISKFPEKDNTVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMG

SEQ ID NO: 59 - protein, 227 aa, Artificial Sequence
  human deltaRI-NBD1
TTTEVVMENVTAFWEEGGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMG

SEQ ID NO: 60 - protein, 8 aa, Artificial Sequence
  C-terminal cleavage site for human rhinovirus 3C (P3C)
LEVLFQGP

SEQ ID NO: 61 - protein, 10 aa, Artificial Sequence
  cMyc tag
EQKLISEEDL

SEQ ID NO: 62 - protein, 28 aa, Artificial Sequence
  Twin-Strep-tag
WSHPQFEKGGGSGGGSGGSAWSHPQFEK

SEQ ID NO: 63 - protein, 121 aa, Artificial Sequence
  T5 Nanobody amino acid sequence
QVQLQESGGGLVQAGGSLRLACVASGNIFTINDMGWYRQAPGKQRELVATITSAGITNYADSVKGRFTISRDNAKNTVFLRMISLKPEDTAVYYCHQAVVHGPIGLEYDYWGQGTQVTVSS

SEQ ID NO: 64 - protein, 127 aa, Artificial Sequence
  S1 Nanobody amino acid sequence
QVQLQESGGGLVQPGGSLRLSCATSRFTLDYGTIGWFRQAPGKEREGVSCIRTSSGSTNYADSVKGRFTIYRDIVKNTIYLQMNSLKPEDTAAYYCAADEARLYGSSCLRMDEYDYWGQGTQVTVSS

SEQ ID NO: 65 - protein, 122 aa, Artificial Sequence
  T29-del Nanobody amino acid sequence
QVQLQESGGGLVQPGDSLRLSCAASGFTMGNYAIGWFRQAPGKEREGIACIGTSAGITNYADSVKGRFTISRDNAKNTVFLRMISLKPEDTAVYYCHQAVVHGPIGLEYDYWGQGTQVTVSS

SEQ ID NO: 66 - protein, 198 aa, Artificial Sequence
  T29 Nanobody amino acid sequence
QVQLQESGGGLVQPGDSLRLSCAASGFTMGNYAIGWFRQAPGKEREGIACIGANDGKTYYSDSVKGRFAASRDNAKSVAYLQESGGGLVQAGGSLRLACVASGNIFTINNMGWYRQAPGKQRELVAFITSAGITNYADSVKGRFTISRDNAKNTVFLRMISLKPEDTAVYYCHQAVVHGPIGLEYDYWGQGTQVTVSS

SEQ ID NO: 67 - protein, 120 aa, Artificial Sequence
  T2a mutant A105F
QVQLQESGGGLVQAGGSLRLSCAASGSIFRIDAMGWYRQAPGKQRELVAHSTSGGSTDYADSVKGRFTISRDNAKNTVYLQMNSLKPEDTAVYYCNADVRTRWYFSNNYWGQGTQVTVSS